(19) United States
(12) Patent Application Publication
(10) Pub. No.: US 2025/0136991 A1
(43) Pub. Date: May 1, 2025

(54) METHOD OF MAKING BONE AND CARTILAGE

(71) Applicants: Seong-Jin Kim, Seoul (KR); Pyunggang Kim, Seoul (KR); Jinah Park, Seoul (KR)

(72) Inventors: Seong-Jin Kim, Seoul (KR); Pyunggang Kim, Seoul (KR); Jinah Park, Seoul (KR)

(21) Appl. No.: 18/835,493

(22) PCT Filed: Feb. 3, 2023
(86) PCT No.: PCT/US2023/012331

§ 371 Date: Aug. 2, 2024

Related U.S. Application Data
(60) Provisional application No. 63/306,677, filed on Feb. 4, 2022.

(51) Int. Cl. C12N 15/113; A61K 38/46; C12N 15/11

(52) U.S. Cl. CPC C12N 15/1137; A61K 38/465; C12N 15/11; C12Y 207/11001; C12N 2310/20; C12N 2310/531

(57) ABSTRACT

The present application describes a method of generating cartilage or bone by controlling level of expression of MAST4 in a cell.

### BACKGROUND OF THE INVENTION

### 1. Field of the Invention

The present invention relates to a therapeutic composition for generating cartilage or bone depending on the regulation of MAST4 protein.

### 2. General Background and State of the Art

Mesenchymal stromal cells (MSCs) are multipotent cells capable of differentiating into various lineages of mesenchymal cell types, including chondrocytes, osteoblasts, and adipocytes¹. The commitment and differentiation of MSCs to each individual cell type depends on a variety of signaling pathways, including Wnt, TGF-β, BMP, and FGF². During differentiation, the coordinated up-regulation and suppression of transcription factors are triggered via specific signaling pathways as well as interactions with other numerous transcription factors that act as co-regulators.

Sox9, a member of the family of high-mobility group (HMG) domain transcription factors, is an activator of chondrogenesis and regulates from the initiation of pre-cartilaginous condensations to the terminal differentiation of chondrocytes³⁻⁵. Sox9 activates collagen genes (Col2, Col9, Col11) and cartilage matrix genes (Acan and Comp) through direct binding on their enhancers and promoters^{6,7}. Considering Sox9 as a key regulator of chondrogenesis, Sox9 is strictly regulated by diverse mechanisms⁸. Several studies have reported phosphorylation events that regulate Sox9 in chondrocytes⁹⁻¹¹.

TGF-β signaling is involved in cartilage development and maintenance, especially stimulating chondrocyte differentiation at the early stage of chondrogenesis^{12,13}. Animal studies have demonstrated that Smad3, a key mediator of TGF-β1 signaling, is required for maintaining articular cartilage, and mice with either Smad3-deficiency or chondrocyte-specific depletion of Smad3 resulted in degeneration of articular cartilage^{14,15}. In addition, previous studies have reported that TGF-β1 signaling facilitates chondrogenesis through regulation of Sox9 in both Smad3-dependent and-independent manners¹⁶⁻¹⁸, implying that TGF-β1-Sox9 axis is critical in regulating chondrogenesis.

Wnt/β-catenin signaling plays a crucial role in endochondral ossification by regulating osteoblast differentiation and maturation¹⁹. Wnt-induced stabilization of intracellular β-catenin and subsequent nuclear translocation leads to the activation of Runx2, a master transcription factor of osteoblast differentiation, especially in mesenchymal cells for development into bone²⁰. Moreover, GSK-3β, a key negative regulator of canonical Wnt/β-catenin signaling, has shown to attenuate Runx2 activity during osteogenesis, suggesting GSK-3β as a potential molecular target for the treatment of bone diseases²¹.

In this work, considering that protein kinases play a crucial role in signal transduction, we have sought to identify a gene that may be involved in the regulation of switching mesenchymal progenitor cells to specific lineages downstream of TGF-β or Wnt signals. Here, we identify that microtubule-associated serine/threonine kinase 4 (Mast4), which is suppressed by TGF-β1 during chondrogenesis of MSCs and enhanced by Wnt-mediated GSK-3β inhibition during osteogenesis of MSCs, plays an essential role in determining the cell fate of MSCs into chondrocyte or osteoblast differentiation. We show that Mast4-induced Sox9 phosphorylation at serine 494 residue results in proteasomal degradation of Sox9. We further demonstrate that Mast4 deficiency leads to increased Sox9 stability and Smad3-Sox9 association, which results in increased transcriptional activity of Sox9 and subsequent expression of chondrocyte marker genes, ultimately facilitating chondrogenic differentiation of MSCs. On the other hand, we find that GSK-3β- induced Mast4 phosphorylation triggers Mast4 recruitment of E3 ligase Smurf1, resulting in Mast4 degradation. We then show that Mast4 stabilized by Wnt-mediated GSK-3β inhibition promotes β-catenin nuclear localization, ultimately increasing Runx2 transcriptional activity and subsequent osteogenic differentiation of MSCs. The effects of Mast4 on chondro-osteogenesis of mesenchymal progenitors are confirmed in vivo by demonstrating excessive cartilage synthesis but osteoporotic or reduced bone formation in Mast4^{-/-} mice. Interestingly, Mast4 depletion in MSCs facilitates cartilage formation and regeneration in vivo. Altogether, our findings uncover essential roles of Mast4 in determining the fate of MSC development into cartilage or bone.

### SUMMARY OF THE INVENTION

The present invention is directed to a method of manipulating MAST4 expression in a cell such that the final product results in the production of cartilage or bone. For instance, if a cell is manipulated such that MAST4 is inhibited, the resultant cell will produce extra cellular matrix material and further if the cells are administered to a site of interest in a subject, cartilage is generated. Conversely, if a cell is manipulated such that MAST4 is highly expressed, and such cells are administered to a site of interest in a subject, bone is generated.

In one aspect, the present invention is directed to a method of generating bone, comprising administering to a subject in need thereof at or near a site of bone defect, where bone is desired to be formed, eukaryotic cells in which expression or activity of Microtubule Associated Serine/Threonine Kinase Family Member 4 (MAST4) protein or a fragment thereof is stabilized or increased compared with normal cell. The method includes recombinantly expressing MAST4 in the cell.

The cell may be a connective tissue cell. The eucaryotic cell may be mesenchymal stem cell, fibroblast, osteoprogenitor cell, osteocyte, preosteoblast, osteoblast or osteoclast. The eucaryotic cell may be allogeneic or autologous with respect to the host. The cell may recombinantly overexpress MAST4 in the cell. The expressed MAST4 may be under control of a viral promoter. The viral promoter may be from lentivirus, or adeno-associated virus.

In another aspect of the invention, the cells may be contacted with a composition comprising (1) a compound that specifically binds to nucleic acid encoding a MAST4 inhibiting protein thus inhibiting expression of the MAST4 inhibiting protein; or (2) a compound that specifically binds to a MAST4 inhibiting protein thus preventing its binding to MAST4. Without being bound by any limitations, the MAST4 inhibiting protein may be GSK-3. The inhibitory compound may be a chemical, polypeptide, or polynucleotide, or a combination thereof. The polypeptide may be an antibody or an antigen-binding molecule. The inhibiting compound of GSK-3alpha or GSK-3beta may be a microRNA (miRNA), small interfering RNA (siRNA), short hairpin RNA (shRNA), Piwi-interacting RNA (piRNA), small nuclear RNA (snRNA), or antisense oligonucleotide, or a combination thereof. The compound may be also CRISPR-Cas comprising guide RNA specific to the nucleic acid encoding the MAST4 inhibiting protein (or the fragment thereof). The guide RNA may be a dual RNA comprising CRISPR RNA (crRNA) and transactivating crRNA (tracrRNA) specific to the nucleic acid encoding the MAST4 inhibiting protein (or the fragment thereof), or a single strand guide RNA comprising parts of the crRNA and the tracrRNA and hybridizing with the nucleic acid encoding the MAST4 inhibiting protein (or the fragment thereof). The cell may be a connective tissue cell. The cell may be mesenchymal stem cell, fibroblast, osteoprogenitor cell, osteocyte, preosteoblast, osteoblast or osteoclast. The cell may be autologous or allogeneic with respect to the host. The cell further may comprise a recombinant construct that expresses MAST4. The recombinant construct may overexpress MAST4.

In another aspect, the invention is directed to a method of producing extracellular matrix from eukaryotic cells, comprising contacting the eukaryotic cells with a composition comprising a compound capable of specifically binding to a nucleic acid encoding Microtubule Associated Serine/Threonine Kinase Family Member 4 (MAST4) protein or a fragment thereof and inhibits expression or activity of the MAST4 protein, wherein the compound capable of specifically binding to the nucleic acid encoding the MAST4 protein or the fragment thereof, wherein the eukaryotic cells are chondrocytes, fibroblasts or mesenchymal stem cells. The inhibitory compound is microRNA (miRNA), small interfering RNA (siRNA), short hairpin RNA (shRNA), Piwi-interacting RNA (piRNA), small nuclear RNA (snRNA), or antisense oligonucleotide, or a combination thereof.

In another aspect, the invention is directed to a method of preventing, treating, or improving a joint disease, the method comprising (i) administering a compound to inhibit Microtubule Associated Serine/Threonine Kinase Family Member 4 (MAST4) in a eukaryotic cell, such that MAST4 protein expression or activity is inhibited; and (ii) administering to a subject in need thereof at or near a joint in need thereof where cartilage is desired to be formed, the eukaryotic cells obtained thereby.

These and other objects of the invention will be more fully understood from the following description of the invention, the referenced drawings attached hereto and the claims appended hereto.

### BRIEF DESCRIPTION OF THE DRAWINGS

The patent or application file contains at least one drawing executed in color. Copies of this patent or patent application publication with color drawings will be provided by the Office upon request and payment of the necessary fee.

The present invention will become more fully understood from the detailed description given herein below, and the accompanying drawings which are given by way of illustration only, and thus are not limitative of the present invention, and wherein;

FIGS. 1a-1f show targeted deletion of the Mast4 gene enhances cartilage matrix gene expression and reduces osteogenic gene expression in vitro. a Representative RT-PCR result, obtained from at least three independent experiments, of high-density micromass culture of C3H10T1/2 cells in the presence of BMP-2. b Representative qRT-PCR result of the expression of chondrocyte marker genes in the differentiating wild-type and Mast4-depleted C3H10T1/2 cells. Mast4 protein expression was confirmed by western blot during chondrogenic differentiation. c Heatmap of DEGs, classified under cartilage/chondrocyte development and osteogenesis, of chondrogenic differentiated wild-type and Mast4-depleted C3H10T1/2 cells for 6 days. d Representative qRT-PCR result of Sox9-targeted genes and Mmp9/13, which were identified by RNA sequencing, in wild-type and Mast4-depleted C3H10T1/2 cells differentiated to chondrocytes for 6 days. e Representative qRT-PCR result of osteoblast marker genes and the genes related to osteogenesis, which were identified in c, in wild-type and Mast4-depleted C3H10T1/2 cells differentiated to osteoblasts for 10 days. f Transcriptional network of the DEGs related to cartilage and chondrocyte development, TGF-β signaling, and BMP signaling in the wild-type and Mast4-depleted C3H10T1/2 cells differentiated to chondrocytes for 6 days. The node size was set on the basis of connectivity of the nodes. Red-colored circles indicate genes showing differential expression in Mast4-depleted C3H10T1/2 cells. b, d and e Data are representative mean±SD of three independent experiments, each conducted in triplicate (n=3). Unpaired two-tailed Student's t test (P<0.05) with Benjamini-Hochberg correction for multiple tests was conducted for all statistical analyses. P values versus WT at corresponding day.

FIGS. 2a-2l show that Mast4 modulates chondrogenesis through post-translational regulation of Sox9. a Alcian blue staining results of C3H10T1/2 cells. b 2×10⁵ of hBMSC were differentiated into chondrocytes for 21 days, followed by protein extraction from the pellets. c C3H10T1/2 cells were differentiated into chondrocytes for 6 days, followed by Sox9 ChIP on Col2a1 gene (TGF-β1 (5 ng/ml) and Vactosertib (0.5 μM), a TGF-β type I receptor kinase inhibitor, for 48 h). d 4xCol2a1-luc, Sox9, and Mast4-PDZ were transiently overexpressed in the wild-type and Mast4-depleted C3H10T1/2 cells, followed by TGF-β1 treatment (3 ng/ml for 24 h). e Mast4-PDZ and Sox9 were transfected to C3H10T1/2 cells, followed by immunoprecipitation assay. f Western blot analysis of Sox9 stability in C3H10T1/2 cells. g Wild-type and Mast4-depleted C3H10T1/2 cells were differentiated to chondrocytes for 6 days, followed by TGF-β1 (5 ng/ml for 48 h) and MG-132 (10 μM for 6 h) treatment, and Sox9 immunoprecipitation. Band intensities representing pSerine and Sox9 were converted by densitometry using ImageJ. Data are reported as mean±SD of three independent experiments (n=3). Unpaired two-tailed Student's t test (P<0.05) was conducted for statistical analysis. h Sox9 and Mast4-PDZ were transfected to C3H10T1/2 cells in the presence of MG-132 (10 μM for 6 h). Two independent MASS SPEC analyses were conducted. i Mast4-PDZ was co-transfected with Sox9 wild-type (WT), S494A, or S494D mutants into C3H10T1/2 cells. j 4xCol2a1-luc and Sox9 WT/S494A/S494D were co-transfected to C3H10T1/2 cells, followed by TGF-β1 treatment (3 ng/ml for 24 h). k Representative alcian blue staining of C3H10T1/2 cells stably overexpressing Sox9 WT, S494A, and S494D in micromass cultures for 6 days. l The wild-type and stably overexpressing Sox9 WT, S494A, and S494D C3H10T1/2 cells were differentiated into chondrocytes for 6 days. The mRNA expression was examined by RT-PCR. c, d and j Data are representative mean±SD of three independent experiments, each conducted in triplicate (n=3). Unpaired two-tailed Student's t test (P<0.05) with Benjamini-Hochberg correction for multiple tests was conducted for all statistical analyses. a, b, e-g, i, and l The representative results were obtained from at least three independent experiments. TCL: total cell lysates.

FIGS. 3a-3g show TGF-β1-induced suppression of Mast4 enhances chondrogenesis by increasing Sox9-Smad3 association. a Sox9 and Smad3 were co-transfected to wild-type and Mast4-depleted C3H10T1/2 cells, followed by TGF-β1 treatment (5 ng/ml for 30 minutes). Sox9 was immunoprecipitated using HA antibody. Band intensities representing GFP-Smad3 and HA-Sox9 expression level in the immunoprecipitates were converted by densitometry using ImageJ software into the ratio of Smad3 to Sox9. Data are reported as mean±SD of three independent experiments (n=3). Unpaired two-tailed Student's t test (P<0.05) was conducted for statistical analysis. b Smad3 ChIP on Col2a1 gene assay was performed in differentiating C3H10T1/2 cells for 6 days. c Mast4 expression was examined by western blot and RT-PCR of C3H10T1/2 cells treated with TGF-β1 for 48 h. d Luciferase assay was conducted in undifferentiated C3H10T1/2 cells. Vactosertib (0.5 μM) was pre-treated for 2 h prior to TGF-β1 (5 ng/ml) treatment for 24 h. e Smad3 ChIP assay on Mast4 gene was conducted in C3H10T1/2 cells undergoing chondrogenic differentiation for 6 days. TGF-β1 (5 ng/ml) and Vactosertib (0.5 μM) were treated for 48 h and 50 h, respectively, before harvest. f Endogenous Mast4 and Sox9 protein expression was examined by western blotting in differentiating C3H10T1/2 cells. g TGF-β1 (5 ng/ml) was treated for 24 h and 48 h, and Vactosertib (0.5 μM) was treated for 48 h to human primary chondrocytes. b, d and e Data are representative mean±SD of three independent experiments, each conducted in triplicate (n=3). Unpaired two-tailed Student's t test (P<0.05) with Benjamini-Hochberg correction for multiple tests was conducted for all statistical analyses. a, c, f and g The representative results were obtained from at least three independent experiments. TCL: total cell lysates.

FIGS. 4a-4l show that Wnt induces osteogenesis by enhancing Mast4 stability through inhibition of GSK-3β. a Representative ALP staining results of osteogenic differentiated C3H10T1/2 cells obtained from at least three independent experiments. b MC3T3-E1 preosteoblasts were treated with Wnt3a conditioned medium for the indicated time. c Wild-type and Mast4-PDZ-overexpressing C3H10T1/2 cells were differentiated into osteoblasts for 10 days. d Mast4-PDZ and GSK-3β were transfected to C3H10T1/2 cells, followed by CHIR-99021 treatment (10 μM for 9 h). e Mast4-PDZ and GSK-3β were transfected to C3H10T1/2 cells, followed by immunoprecipitation assay. The bands, which were recognized by phospho-serine antibody, were later reprobed with HA antibody. f Smurf1, Mast4-PDZ and GSK-3β were transfected to C3H10T/12 cells treated with CHIR-99021 treatment (10 μM for 9 h), followed by immunoprecipitation assay. g Smurf1 and GSK-3β were transfected to wild-type and GSK-3β-depleted Mast4-PDZ-overexpressing C3H10T1/2 cells. h Mast4-PDZ WT, P628A/Y634A and Smurf1 were transfected to wild-type and GSK-3β- depleted C3H10T1/2 cells. i Various Mast4 kinase domain deletion mutants and GSK-3β were transfected to C3H10T1/2 cells. j Flag-Mast4-PDZ WT and Δ632-636 were co-transfected into C3H10T1/2 cells together with HA-Ubiquitin in the absence or presence of Smurf1 and GSK-3β. Cell lysates were immunoprecipitated with Flag antibody and immunoblotted with the indicated antibodies. k MC3T3-E1 cells were transfected with Mast4-PDZ WT and Δ632-636, followed by treatment of Wnt3a conditioned medium (2.5 h). l 6xOSE-Luc, Mast4-PDZ WT and Δ632-636 were transfected to C3H10T1/2 cells, followed by treatment of Wnt3a conditioned medium (18 h). Data are representative mean±SD of three independent experiments, each conducted in triplicate (n=3). Unpaired two-tailed Student's t test (P<0.05) with Benjamini-Hochberg correction for multiple tests was conducted for all statistical analyses. b-k The representative results were obtained from at least three independent experiments. TCL: total cell lysates.

FIGS. 5a-5h show Mast4 depletion induces altered chondrogenesis and osteogenesis during development. a, b Immunofluorescence images of Sox9 and Col2a1 in the growth plate of Mast4^{+/+} and Mast4^{-/-} mice at PN 1 day (n=3). c, d Pentachrome staining of the tibial growth plates of the (c) 1-day-old and (d) 3-week-old Mast4^{+/+} and Mast4^{-/-} mice (n=5). Black arrows indicate the hypertrophic zone. e The μCT images of the trabecular bone in the tibias of Mast4^{+/+} and Mast4^{-/-} mice. Total bone volume and bone mineral density (BMD) of trabecular bone were measured from the μCT images (n=8 for Mast4^{+/+} and 10 for Mast4^{-/-} mice). f Bone formation was visualized by double calcein labelling at 7 d and 2 d prior to sacrifice, and the distance between labels were measured at 3 points per tibia (n=10 tibias for Mast4^{+/+} and 5 tibias Mast4^{-/-} mice). MAR: mineral apposition rate. g Representative images of the immunofluorescence staining of Osterix and Mmp13 near metaphyseal blood vessels (CD31) in the proximal tibial of 6-week-old Mast4^{+/+} and Mast4^{-/-} mice (n=3). GP: growth plate, MP: metaphysis. h Representative alcian blue and Alizarin Red S staining results obtained from in vitro colony formation assay using skeletal stem cells isolated from 5-week-old Mast4^{+/+} mice and Mast4^{-/-} mice (n=5). e, f, Data are reported as mean±SD. Unpaired two-tailed Student's t test (P<0.05) was conducted for all statistical analyses.

FIGS. 6a-6e show identification of the genes regulated by Mast4 in mouse cartilage and bone. a, b RNA sequencing was conducted by collecting and combining RNAs obtained from cartilage and bone of the tibias of Mast4^{+/+} and Mast4^{-/-} mice at PN 1 day (3 mice per each group). a The enrichment of up-regulated genes associated with cartilage development in the cartilage of Mast4^{-/-} mice. Twenty up-regulated genes were predicted as the leading-edge subset of the enriched gene set. b The enrichment of up-regulated genes associated with skeletal system development and Wnt signaling pathway in the bone of Mast4^{+/+} mice. In the plot, 20 and 12 genes were up-regulated as the leading-edge subsets of the enriched gene sets in Mast4^{+/+} mice. Gene set enrichment analysis was applied with a background dataset consisting of all DEGs analyzed from cartilage and bone of the tibias of Mast4^{+/+} mice and Mast4^{-/-} mice. c Mast4-regulated transcriptional network of Sox9 and Runx2 targets showing differential expression in cartilage and bone. The Sox9 and Runx2 targets are linked to the genes related to the functions of skeletal system development, including cartilage and bone development, TGF-β signaling, BMP signaling and Wnt signaling. d Western blot analysis of Col2a1 and Sox9 proteins in the cartilage tissues of Mast4^{+/+} mice and Mast4^{-/-} mice at PN 1 day. e Western blot analysis of Runx2, Mmp13 and β-catenin proteins in the bone tissues of Mast4^{+/+} mice and Mast4^{-/-} mice at PN 1 day. d, e The representative results were obtained from at least three independent experiments.

FIGS. 7a-7d show transplantation of Mast4-depleted MSCs improves cartilage formation and repair in vivo. a A hundred micromass cultures of the wild-type and Mast4-depleted C3H10T1/2 cells cultured in a 150 mm dish with chondrogenic differentiation medium for 4 days were subcutaneously injected into each side of the flanks of athymic nude mice (n=4; WT on the left flank and KO on the right flank). Ectopic masses were retrieved at 2 weeks after implantation. The values given are mean±SD of the volume of grafts from 4 mice. Unpaired two-tailed Student's t test (P<0.05) was conducted for statistical analysis for (a). b Multiple cartilage structures found in the grafts of wild-type and Mast4-depleted C3H10T1/2 cells were confirmed by immunofluorescence staining of Col2a1 and Sox9. Merge of Sox9 and TOPRO-3 is shown in yellow. The representative images were obtained from immunostainings of 1 cartilaginous node formed by wild-type C3H10T1/2 cells and 7 different cartilaginous nodes formed by Mast4-depleted C3H10T1/2 cells. c Gross and microscopic appearances of full-thickness cartilage defects in the trochlear groove in a rabbit model at 12 weeks post-transplantation. Vehicle: PBS treatment without hBMSC transplantation. d Wakitani cartilage repair scores of the regenerated cartilage. Data are represented as mean±SD. c, d n=3 for vehicle-treated group, n=5 for naïve and MAST4-depleted hBMSC-transplanted groups. One-way ANOVA with Dunnett's correction for multiple comparisons was conducted for statistical analyses for (d).

FIG. 8 shows gene expression during chondrogenesis of ATDC5 cells. ATDC5 cells were treated with insulin 100 ng/ml for 6 and 9 days to induce chondrogenesis, and RT-PCR was conducted. The representative result were obtained from at least three separate experiments.

FIGS. 9a-9e show the effect of Mast4 depletion on the mRNA expression of chondrocyte marker genes. (a) Analysis of CRISPR/Cas9-mediated deletion of Mast4 exon 1 and exon 2 in C3H10T1/2 cells. Exon 1 and 2 of Mast4 locus were amplified to distinguish genotypes, and PCR products were used for sequencing. Two bases were deleted in both strands of exon 1, inducing a nonsense mutation with translational stop in exon 1. Seven-base deletion and one-base insertion were found in exon 2, inducing a biallelic mutation with translational stop in exon 2. (b) Depletion of Mast4 protein was confirmed by western blot by using the undifferentiated cells. (c and d) Expression of chondrocyte marker genes in the undifferentiated (c) wild-type and Mast4-depleted, (d) control and shRNA-mediated Mast4 knockdown C3H10T1/2 cells. (d) Data are representative mean±SD of three independent experiments, each conducted in triplicate (n=3). Unpaired two-tailed Student's t test (P<0.05) with Benjamini-Hochberg correction for multiple tests was conducted for statistical analysis. P values versus WT. (e) Expression of chondrocyte marker genes was examined in wild-type and Mast4-depleted C3H10T1/2 cells that were induced to chondrogenic differentiation for 6 days. (b, c, and e) The representative result were obtained from at least three separate experiments.

FIGS. 10a-10b show Sox9 expression in wild-type and Mast4-depleted C3H10T1/2 cells. (a) Endogenous Sox9 protein expression was examined in the undifferentiated wild-type and Mast4-depleted C3H10T1/2 cells. Band intensities representing Sox9 expression level were converted by densitometry using ImageJ software into the ratio of Sox9 to b-actin. The representative result were obtained from at least three separate experiments. (b) The mRNA expression of Sox9 in the undifferentiated wild-type and Mast4-depleted C3H10T1/2 cells was examined by qRT-PCR. Data are representative mean±SD of three independent experiments, each conducted in triplicate (n=3). Unpaired two-tailed Student's t test (P<0.05) with Benjamini-Hochberg correction for multiple tests was conducted for statistical analysis.

FIGS. 11a-11b show forced expression of a truncated Mast4 protein in C3H10T1/2 cells. (a) Schematic structures of full-length and truncated Mast4 constructs. (b) RT-PCR was performed using control (LPCX) and Mast4-PDZ-overexpressing C3H10T1/2 cells to analyze mRNA expression of chondrocyte marker genes. The representative result were obtained from at least three separate experiments.

FIG. 12 shows the DEG and GO enrichment analysis of WT and Mast4-depleted C3H10T1/2 cells. The wild-type and Mast4-depleted (KO #1) C3H10T1/2 cells were differentiated into chondrocytes for 6 days under BMP-2 stimulation in high-density micromass cultures, followed by RNA sequencing analysis. Differentially expressed genes (DEG) analysis and gene ontology (GO) enrichment analysis were conducted.

FIG. 13 shows expression of collagen gene family in wild-type and Mast4-depleted C3H10T1/2 cells. RNA sequencing shows changes in expression of collagen gene family in wild-type and Mast4-depleted C3H10T1/2 cells.

FIGS. 14a-14e show the effect of Mast4 depletion in C3H10T1/2 cells upon chondrogenesis in vitro. (a) The schematic representation of chondrogenic differentiation of C3H10T1/2 cells in a high-density micromass culture system. (b) 5×10³ of the undifferentiated wild-type and Mast4-depleted C3H10T1/2 cells were plated on 6-well plate. Two micromass cultures (each mass: 1×10⁵/10 ml droplet) were plated on 12-well plate, following chondrogenic differentiation under BMP-2 stimulation. The number of cells was counted with Countess II automated cell counter at the indicated time points. Data are representative mean±SD of three independent experiments, each conducted in triplicate (n=3). Unpaired two-tailed Student's t test (P<0.05) with Benjamini-Hochberg correction for multiple tests was conducted for all statistical analyses. P values versus WT. (c) Representative alcian blue staining results were obtained from at least three separate experiments using chondrogenic differentiated C3H10T1/2 cells. (d) 1×10⁵ of the wild-type and Mast4-depleted C3H10T1/2 cells in 50 ml volume were plated on a low-binding 96-well plate, following chondrogenic differentiation for 8 days. Representative phase-contrast microscopy images of 3D spheroids were obtained from at least three separate experiments. (e) The mRNA was isolated from the spheroids shown in (d), and the expression of chondrogenic marker genes was analyzed by RT-PCR. (c and e) The representative result were obtained from at least three separate experiments.

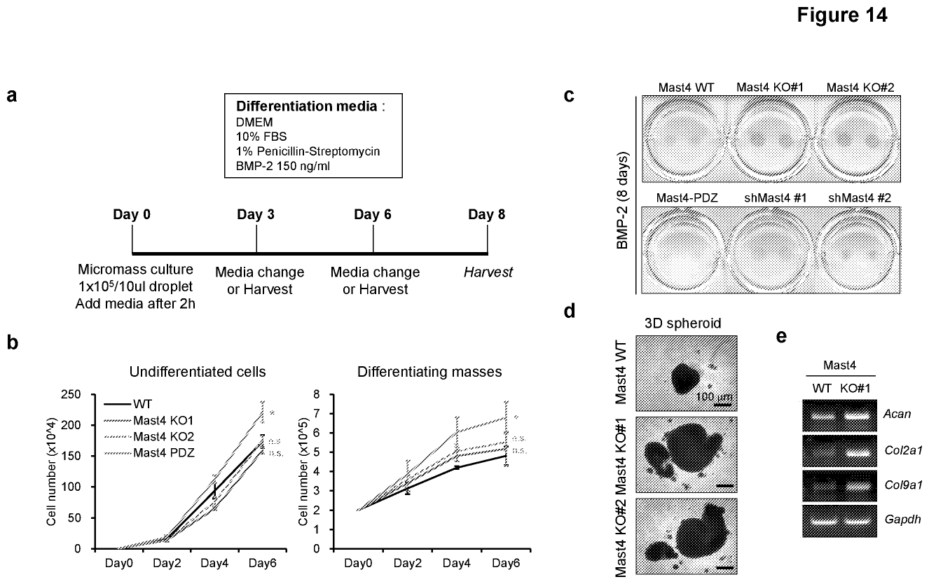

FIGS. 15a-15b show chondrogenic differentiation of human bone marrow-derived stem cells (hBMSCs). (a) The schematic representation of chondrogenic differentiation of human bone marrow-derived stem cells (hBMSCs) in a pellet culture system. (b) 2×10⁵ of hBMSCs were differentiated into chondrocytes for 21 days under pellet culture system, followed by mRNA isolation from the aggregates. The mRNA expression of Col2a1 and Acan was examined by qRT-PCR. Undif.: undifferentiated hBMSC. Data are representative mean±SD of three independent experiments, each conducted in triplicate (n=3). Unpaired two-tailed Student's t test (P<0.05) with Benjamini-Hochberg correction for multiple tests was conducted for all statistical analyses.

FIGS. 16a-16e show that Mast4 regulates Sox9 binding to Col2a1 and Sox9 stability. (a) The indicated C3H10T1/2 cells were differentiated into chondrocytes for 6 days, followed by Sox9 ChIP on the Col2a1 gene. (b) 4xCol2a1-luc was transiently overexpressed in LPCX control and Mast4 PDZ-overexpressing C3H10T1/2 cells, followed by TGF-b1 treatment (3 ng/ml for 24 h). (a and b) Data are representative mean±SD of three independent experiments, each conducted in triplicate (n=3). Unpaired two-tailed Student's t test (P<0.05) with Benjamini-Hochberg correction for multiple tests was conducted for all statistical analyses. P values versus WT. (c) Full-length (HA-Mast4-FULL) and truncated Mast4 (HA-Mast4-PDZ) together with siMAST4 (50 pmol) that targets exogenous Mast4 (human) were co-transfected with Myc-Sox9 into C3H10T1/2 cells. Due to inefficient detection of full length Mast4 with HA antibody, Mast4 antibody which recognizes the C-terminus was used. (d) The indicated C3H10T1/2 cells were differentiated to chondrocytes for 6 days, followed by MG-132 (10 mM for 6 h) treatment. Endogenous Sox9 was immunoprecipitated, and the complexes were analyzed by western blot using phospho-serine and Sox9 antibodies. (e) Myc-Sox9 and Mast4-PDZ constructs were transfected to C3H10T1/2 cells, followed by MG-132 (10 mM for 6 h) treatment. (c-e) Band intensities representing Myc-Sox9 or serine-phosphorylated Sox9 expression level were converted by densitometry using ImageJ software into the ratio of Myc-Sox9 or pSerine to b-actin or Sox9 in the immunoprecipitates. The representative result were obtained from at least three separate experiments.

FIG. 17 shows that Mast4 regulation of Sox9 through phosphorylation at serine 494 of Sox9. 4xCol2a1-luc, Sox9 WT/S494A/S494D, and Mast4-PDZ were transiently overexpressed in wild-type and Mast4-depleted C3H10T1/2 cells as indicated. Data are representative mean±SD of three independent experiments, each conducted in triplicate (n=3). Unpaired two-tailed Student's t test (P<0.05) with Benjamini-Hochberg correction for multiple tests was conducted for statistical analysis.

FIGS. 18a-18c shows Mast4 interaction with Smad3 and their effect on the TGF-b1/Smad3-induced transcriptional activation. (a) The indicated plasmids were co-transfected to C3H10T1/2 cells, followed by immunoprecipitation assay. The representative result were obtained from at least three separate experiments. (b and c) Smad3/4-induced transcriptional activation was examined by (b) CAGA and (c) SBE reporter assay in wild-type and Mast4-depleted C3H10T1/2 cells. Data are representative mean±SD of three independent experiments, each conducted in triplicate (n=3). Unpaired two-tailed Student's t test (P<0.05) with Benjamini-Hochberg correction for multiple tests was conducted for all statistical analyses.

FIGS. 19a-19b show the effect of Mast4 on Smad3 binding to Smad7 and TGF-b1 target gene expression. (a) Smad3 ChIP assay examining Smad3 binding to Smad7 was conducted in the wild-type and Mast4-depleted differentiating C3H10T1/2 cells. Data are representative mean±SD of three independent experiments, each conducted in triplicate (n=3). Unpaired two-tailed Student's t test (P<0.05) with Benjamini-Hochberg correction for multiple tests was conducted for all statistical analyses. (b) The mRNA expression of TGF-b1 target genes was examined in the wild-type (WT), Mast4-depleted (KO; KO #1), and Mast4-PDZ-overexpressing (OE) C3H10T1/2 cells.

FIGS. 20a-20b show examination of TGF-b1 regulation of Mast4 promoter activity and Smad3 binding to the Mast4 promoter through Smad3 ChIP assay. (a) The expected binding sites of Smad3/4, p300, c-Jun, Nkx2.1, and E2F-4 transcription factors in the Mast4 promoter were predicted using PROMO v3.0.2 software. (b) Smad3 ChIP assay on Mast4 gene was performed in differentiating (6 days in chondrogenic differentiation medium) C3H10T1/2 cells in the presence or absence of TGF-b1 (5 ng/ml for 48 h). Data are representative mean±SD of three independent experiments, each conducted in triplicate (n=3). Unpaired two-tailed Student's t test (P<0.05) with Benjamini-Hochberg correction for multiple tests was conducted for all statistical analyses.

FIG. 21 shows mRNA expression of Mast4 and chondrocyte marker genes in differentiating C3H10T1/2 cells treated with vactosertib. Vactosertib (0.5 mM) was treated for 3 and 6 days in differentiating C3H10T1/2 cells. The mRNA expression of chondrocyte marker gene expression was examined by RT-PCR. The representative result were obtained from at least three separate experiments.

FIGS. 22a-22e show Mast4 induction during osteogenesis and GSK-3b regulation of Mast4 expression. (a) The schematic representation of osteogenic differentiation of C3H10T1/2 cells. (b) The indicated C3H10T1/2 cells were differentiated into osteoblasts for 10 days, followed by Alizarin Red S staining. (c) C3H10T1/2 cells were differentiated into osteoblasts for 10 days, followed by western blot analysis for the expression of Mast4 and key Wnt signaling molecules. (d) Mast4-PDZ-overexpressing C3H10T1/2 cells were treated with cycloheximide (CHX; 10 mg/ml) for the indicated time in the absence or presence of CHIR-99021 (10 mM for 9 h). (e) The wild-type and GSK-3b-depleted HA-Mast4-PDZ-overexpressing C3H10T1/2 cells were treated with MG-132 (10 mM for 4 h) and CHIR-99021 (10 mM for 9 h). (b-d) The representative result were obtained from at least three separate experiments.

FIGS. 23a-23c show GSK-3b and Smurf1 binding to the kinase domain of Mast4. (a) Schematic representation of Mast4-PDZ deletion mutants. (b) GFP-GSK-3b, (c) GFP-Smurf1 and various deletion mutants of Mast4 were co-transfected into C3H10T1/2 cells, followed by immunoprecipitation assay. The representative result were obtained from at least three separate experiments.

FIGS. 24a-24b show enhanced Runx2 activity and osteogenic differentiation by Mast4-PDZ overexpression and GSK-3b depletion. (a) 6xOSE-Luc was transfected to wild-type and GSK-3b-depleted wild-type and Mast4-PDZ-overexpressing C3H10T/12 cells, followed by treatment with Wnt3a conditioned medium for 18 h. (b) The wild-type and GSK-3b-depleted wild-type and Mast4-PDZ-overexpressing C3H10T/12 cells were differentiated to osteoblasts for 10 days, followed by examination of mRNA expression of osteoblast marker genes. (a and b) Data are representative mean±SD of three independent experiments, each conducted in triplicate (n=3). Unpaired two-tailed Student's t test (P<0.05) with Benjamini-Hochberg correction for multiple tests was conducted for all statistical analyses.

FIGS. 25a-25e show analysis of CRISPR/Cas9-mediated deletion of Mast4 exon 1 and exon 15. (a) The mouse Mast4 locus is depicted with boxes of exons. The structure of the Mast4^{-/-} allele is shown. (b-c) Exon 1 and 15 of Mast4 were amplified to distinguish genotypes, and PCR products were used for sequencing. As shown in the sequencing results, 71 bases were deleted in exon 1, resulting in a nonsense mutation with translational stop in exon1; while 3 bases were deleted in exon 15, removing one arginine. (d) Loss of Mast4 proteins in Mast4^{-/-} mice was confirmed by western blot analysis in the brain and muscle of young mice. (e) Representative image showing the body size of Mast4^{+/+}, Mast4^{+/-} and Mast4^{-/-} mice at 6 weeks of age. (b and d) The representative result were obtained from at least three separate experiments.

FIGS. 26a-26e show analysis of Mast4 expression pattern, the tibial growth plate thickness and hypertrophic zone ratio of Mast4^{+/+} mice and Mast4^{-/-} mice. (a) Expression pattern of Mast4 in the tibias of PN 1 day Mast4^{+/+} mice and Mast4^{-/-} mice (n=3). The white dotted lines show the boundaries between the proliferating, hypertrophic, and ossification zones. P: proliferating zone, H: hypertrophic zone, O: ossification zone. (b) Pentachrome- and H&E-stained images of tibial growth plates (n=3). (a and b) The representative images were obtained from immunostainings of Mast4^{+/+} mice and Mast4^{-/-} mice (n=3). (c-e) Total growth plate thickness and hypertrophic zone thickness were measured at 3 random fields of each sample (PN 1 day: n=4, PN 3 weeks: n=5, PN 6 weeks: n=3 for both Mast4^{+/+} and Mast4^{-/-} mice). Hypertrophic zone ratio was calculated as hypertrophic zone thickness/growth plate thickness. Data are reported as mean±SD. Unpaired two-tailed Student's t test (P<0.05) was conducted for statistical analyses for (c, d, e). P values versus WT.

FIGS. 27a-27d show mCT analysis, elemental mapping by an electron probe microanalyzer and limb length of Mast4^{+/+} and Mast4^{-/-} mice. (a) The reconstructed 3-dimensional mCT images of the tibias. The representative images were obtained from immunostainings of Mast4^{+/+}, Mast4+/− and Mast4^{-/-} mice (n=3). (b) The bone volume, bone mineral density and thickness of the cortical bone in the tibias of the 6-week-old Mast4^{+/+} mice and Mast4^{-/-} mice were examined using the mCT images. (c) Elemental mapping of calcium (Ca), phosphorus (P), and magnesium (Mg) in the tibias of the 6-week-old Mast4^{+/+} mice and Mast4^{-/-} mice. The portion colored in red indicates high elemental concentration, while the dark blue indicates low concentration. BEI: Backscattered electron image. (d) The length of limbs of the 6-week-old Mast4^{+/+} and Mast4^{-/-} mice was examined using the mCT images. A cross section including the axis passing through the two points furthest from the bone was set, and the longest length was measured. (b and d) Data are reported as mean±SD (n=8 for Mast4^{+/+} mice and n=10 for Mast4^{-/-} mice). Unpaired two-tailed Student's t test (P<0.05) was conducted for statistical analyses for (b, d). P values versus WT.

FIGS. 28a-28b show expression of osteoblast marker proteins in the proximal tibias and distal femurs of 6-week-old Mast4^{+/+} and Mast4^{-/-} mice. (a) Expression of Runx2 in the proximal tibial metaphysis of Mast4^{+/+} and Mast4^{-/-} mice. GP: Growth plate, MP: Metaphysis. The images showing the expression of Osterix and Mmp13 correspond to FIG. 5g. (b) Expression of Runx2, Osterix, and Mmp13 are reduced in the periosteum of 6-week-old Mast4^{-/-} mice distal femur. P: Periosteum, C: Cortex, M: Medulla. (a and b) The representative images were obtained from immunostainings of Mast4^{+/+} mice and Mast4^{-/-} mice (n=3).

FIGS. 29a-29b show isolation of mouse skeletal stem cells from 5-week-old Mast4^{+/+} and Mast4^{-/-} mice. (a) Representative FACS plots with the percentages of parent gate for each population, as generated on the BD FACS Aria II and analyzed by FlowJo v10.7.1 and BD FACSDiva v.9.0.1 software. (b) The antibodies and concentrations used to isolate mouse skeletal stem cells.

FIGS. 30a-30c show functional assessment of mouse skeletal stem cells isolated from Mast4^{+/+} mice and Mast4^{-/-} mice. (a) The schematic representation of in vitro colony formation assay of mouse skeletal stem cells. (b and c) The stained colonies were extracted for absorbance measurements by using cloning cylinder to distinguish each colony. Alcian blue and Alizarin Red S stainings were quantified by absorbance measurement at 630 nm and 405 nm, respectively. Data are reported as mean±SD of three stained colonies (n=3). Unpaired two-tailed Student's t test (P<0.05) was conducted for statistical analyses for (b, c).

FIGS. 31a-31d show chondrogenic and osteogenic differentiation of mouse bone marrow-derived mesenchymal stem cells (mBMMSCs). (a) The schematic representation of mouse bone marrow-derived mesenchymal stem cells (mBMMSCs) that were differentiated into either chondrocytes or osteocytes. (b) Mouse BMMSCs were isolated from an aspirate of bone marrow harvested from the tibia marrow compartments of Mast4^{+/+} mice and Mast4^{-/-} mice, followed by induction of chondrogenic differentiation for 21 days and subsequent alcian blue staining (n=3). (c) Expression of chondrocyte marker genes in the differentiated BMMSCs shown in (b). (d) The mBMMSCs isolated from Mast4^{+/+} and Mast4^{-/-} mice were cultured in osteoblastic culture medium and stained with alkaline phosphatase (ALP). (c and d) The representative result were obtained from at least three separate experiments.

FIGS. 32a-32b show identification of genes regulated by Mast4 in the mouse cartilage and bone. (a) DEGs were classified into five clusters based on the changes of gene expression in the cartilage and bone. (b) GO enrichment analysis for DEGs within the 5 clusters. A˜M indicate enriched GO terms (P<0.001). The box plot in (a) shows enrichment scores for GO terms described in (b). The grayscale heatmap in (a) indicates DEGs hit to GO terms.

FIGS. 33a-33d show Mast4 regulation of Sox9 target genes and osteogenesis-associated genes in the mouse cartilages and bones, respectively. (a and c) Heatmap and (b and d) qRT-PCR validation of mRNA expression of the Sox9 target genes in the cartilage tissues and the osteogenesis-associated genes in the bone tissues of the tibia of Mast4^{+/+} mice and Mast4^{-/-} mice at PN 1 day. (b and d) Data are representative mean±SD of three independent experiments, each conducted in triplicate (n=3). Unpaired two-tailed Student's t test (P<0.05) with Benjamini-Hochberg correction for multiple tests was conducted for all statistical analyses.

FIGS. 34a-34b show effect of Mast4 depletion in C3H10T1/2 cells upon cartilage formation in vivo. (a) The schematic representation of the preparation of the differentiating C3H10T1/2 cells for subcutaneous injection into the flanks of 4 athymic nude mice (6-week-old females). Mice were sacrificed after 2 weeks, and the grafts were retrieved for further analyses. (b) Multiple cartilage structures found in the masses formed by Mast4-depleted C3H10T1/2 cells were confirmed by pentachrome staining. The image in each rectangle is presented at a larger magnification. Bar=100 mm. The representative images were obtained from immunostainings of a cartilaginous node formed by wild-type C3H10T1/2 cells and 7 different cartilaginous nodes formed by Mast4-depleted C3H10T1/2 cells.

FIGS. 35a-35c show transplantation of hBMSCs into full-thickness cartilage defects in a rabbit model. (a) Gross appearance of full-thickness cartilage defects in a rabbit model and microscopic appearance of H&E, Safranin O, Masson's trichrome, and immunohistochemical detection of type II collagen and human HLA class 1 ABC of the defects in the trochlear groove at 12 weeks post-transplantation. Vehicle: PBS treatment without hBMSC transplantation. The representative images obtained from n=3 for vehicle-treated group, n=5 for naïve and MAST4-depleted hBMSC-transplanted groups. (b) The average percentage of the collagen area out of entire area of cartilage defect was assessed with the tissues stained with Masson's trichrome. (c) The average percentage of the type II collagen-stained area out of entire area of cartilage defect. (b and c) n=3 for vehicle, n=5 for Naïve and CRISPR/Cas9-mediated MAST4 KO (pool) human bone marrow-derived stem cells. Data are represented as mean±SD. Statistical analyses for (b, c) were performed using one-way analysis of variance (ANOVA) with Dunnett's correction for multiple comparisons.

FIGS. 36a-36b show histological grading of the regenerated cartilage at the full-thickness articular cartilage defect sites in rabbit knee. (a) Wakitani cartilage repair scoring system. (b) The sections were semi-quantitatively analyzed with a Wakitani score. (n=3 for vehicle, n=5 for Naïve and MAST4 KO (pool) human bone marrow-derived stem cells). Data are represented as mean±SD. Statistical analyses for (b) were performed using one-way analysis of variance (ANOVA) with Dunnett's correction for multiple comparisons.

FIG. 37 shows schematic diagram for the role of Mast4 in determining the cell fate of MSC development into cartilage or bone. During chondrogenesis, TGF-β1-mediated suppression of Mast4 leads to the increase of Sox9 protein by decreasing Sox9 phosphorylation at S494, which result in increased Sox9 transcriptional activity, ultimately causing MSCs to favor chondrogenesis at the expense of bone formation. During osteogenesis, Wnt-mediated GSK-3b inhibition blocks GSK-3b-induced Mast4 phosphorylation and subsequent Smurf1-mediated Mast4 degradation. The stabilized Mast4 induces an increase in b-catenin and Runx2 activity, resulting in enhanced osteogenesis of MSCs.

### DETAILED DESCRIPTION OF THE PREFERRED EMBODIMENTS

In the present application, “a” and “an” are used to refer to both single and a plurality of objects.

As used herein, administration “in combination with” one or more further therapeutic agents includes simultaneous (concurrent) or consecutive administration in any order.

As used herein, the term “biologically active” in reference to a nucleic acid, protein, protein fragment or derivative thereof is defined as an ability of the nucleic acid or amino acid sequence to mimic a known biological function elicited by the wild type form of the nucleic acid or protein.

As used herein, the term “bone growth” relates to bone mass. This is suggested by the increase in the number and size of osteoblasts, and increased deposition of osteoid lining bone surfaces following systemic administration.

As used herein, “carriers” include pharmaceutically acceptable carriers, excipients, or stabilizers which are nontoxic to the cell or mammal being exposed thereto at the dosages and concentrations employed. Often, the pharmaceutically acceptable carrier is an aqueous pH buffered solution. Examples of pharmaceutically acceptable carriers include without limitation buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

As used herein, the term “connective tissue” is any tissue that connects and supports other tissues or organs, and includes but is not limited to a ligament, a cartilage, a tendon, a bone, or a synovium of a mammalian host.

As used herein, the term “connective tissue cell” or “cell of a connective tissue” include cells that are found in the connective tissue, such as fibroblasts, cartilage cells (chondrocytes), and bone cells (osteoblasts/osteocytes), as well as fat cells (adipocytes) and smooth muscle cells. Preferably, the connective tissue cells are fibroblasts, chondrocytes, and bone cells. More preferably, the connective tissue cells are fibroblast cells. Alternatively, the connective tissue cells are osteoblast or osteocytes. It will be recognized that the invention can be practiced with a mixed culture of connective tissue cells, as well as cells of a single type. It is also recognized that the tissue cells may be treated such as by chemical or radiation so that the cells stably express the gene of interest. Preferably, the connective tissue cell does not cause a negative immune response when injected into the host organism. It is understood that allogeneic cells may be used in this regard, as well as autologous cells for cell-mediated gene therapy or somatic cell therapy.

As used herein, “connective tissue cell line” includes a plurality of connective tissue cells originating from a common parent cell.

As used herein, “host cell” includes an individual cell or cell culture which can be or has been a recipient of a vector of this invention. Host cells include progeny of a single host cell, and the progeny may not necessarily be completely identical (in morphology or in total DNA complement) to the original parent cell due to natural, accidental, or deliberate mutation and/or change.

As used herein, the term, “low bone mass” refers to a condition where the level of bone mass is below the age specific normal as defined in standards by the World Health Organization “Assessment of Fracture Risk and its Application to Screening for Postmenopausal Osteoporosis (1994). Report of a World Health Organization Study Group. World Health Organization Technical Series 843”, which is incorporated by reference herein in its reference to normal and osteoporotic levels of bone mass. Further, the term “bone mass” refers to bone mass per unit area, which is sometimes referred to as bone mineral density.

As used herein, the term “mammalian host” includes members of the animal kingdom including but not limited to human beings.

As used herein, the term “mature bone” relates to bone that is mineralized, in contrast to non-mineralized bone such as osteoid.

As used herein, the term “osteogenically effective” means that amount which effects the formation and development of mature bone.

As used herein, the term “osteoprogenitor cells” or “bone progenitor cells” are cells that have the potential to become bone cells, and reside in the periosteum and the marrow. Osteoprogenitor cells are derived from connective tissue progenitor cells that reside also in the surrounding tissue (muscle).

As used herein, the term “patient” includes members of the animal kingdom including but not limited to human beings.

As used herein, a composition is “pharmacologically or physiologically acceptable” if its administration can be tolerated by a recipient animal and is otherwise suitable for administration to that animal. Such an agent is said to be administered in a “therapeutically effective amount” if the amount administered is physiologically significant. An agent is physiologically significant if its presence results in a detectable change in the physiology of a recipient patient.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

As used herein, a “promoter” can be any sequence of DNA that is active, and controls transcription in an eucaryotic cell. The promoter may be active in either or both eucaryotic and procaryotic cells. Preferably, the promoter is active in mammalian cells. The promoter may be constitutively expressed or inducible. Preferably, the promoter is inducible. Preferably, the promoter is inducible by an external stimulus. More preferably, the promoter is inducible by hormones or metals. Likewise, “enhancer elements”, which also control transcription, can be inserted into the DNA vector construct, and used with the construct of the present invention to enhance the expression of the gene of interest.

As used herein, “subject” is a vertebrate, preferably a mammal, more preferably a human.

As used herein, a “dose” refers to a specified quantity of a therapeutic agent prescribed to be taken at one time or at stated intervals.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” refers to both therapeutic treatment and prophylactic or preventative measures. Those in need of treatment include those already with the disorder as well as those in which the disorder is to be prevented. “Palliating” a disease means that the extent and/or undesirable clinical manifestations of a disease state are lessened and/or the time course of the progression is slowed or lengthened, as compared to a situation without treatment.

As used herein, “vector”, “polynucleotide vector”, “construct” and “polynucleotide construct” are used interchangeably herein. A polynucleotide vector of this invention may be in any of several forms, including, but not limited to, RNA, DNA, RNA encapsulated in a retroviral coat, DNA encapsulated in an adenovirus coat, DNA packaged in another viral or viral-like form (such as herpes simplex, and adeno-associated virus (AAV)), DNA encapsulated in liposomes, DNA complexed with polylysine, complexed with synthetic polycationic molecules, complexed with compounds such as polyethylene glycol (PEG) to immunologically “mask” the molecule and/or increase half-life, or conjugated to a non-viral protein. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

The term “antibody” means a specific immunoglobulin directed against an antigenic site. A gene of interest, such as encoding GSK-3alpha or GSK-3beta, is cloned into an expression vector to obtain the protein encoded by the gene, and the antibody may be prepared from the protein according to a common method in the art. A type of the antibody includes a polyclonal antibody or a monoclonal antibody, and includes all immunoglobulin antibodies. The antibody includes not only complete forms having two full-length light chains and two full-length heavy chains but also functional fragments of antibody molecules which have a specific antigen binding site (binding domain) directed against an antigenic site to retain an antigen-binding function, although they do not have the intact complete antibody structure having two light chains and two heavy chains.

The term “polynucleotide” may be used in the same meaning as a nucleotide or a nucleic acid, unless otherwise mentioned, and refers to a deoxyribonucleotide or a ribonucleotide. The polynucleotide may include an analog of a natural nucleotide and an analog having a modified sugar or base moiety, unless otherwise mentioned. The polynucleotide may be modified by various methods known in the art, as needed. Examples of the modification may include methylation, capping, substitution of a natural nucleotide with one or more homologues, and modification between nucleotides, for example, modification to uncharged linkages (e.g., methylphosphonate, phosphotriester, phosphoroamidate, carbamate, etc.) or charged linkages (e.g., phosphorothioate, phosphorodithioate, etc.).

In a specific embodiment, as the compound capable of specifically binding to the nucleic acid encoding the protein of interest or the fragment thereof, the polynucleotide capable of specifically binding to the nucleic acid encoding the protein of interest or the fragment thereof may be microRNA (miRNA), small interfering RNA (siRNA), short hairpin RNA (shRNA), Piwi-interacting RNA (piRNA), small nuclear RNA (snRNA), or antisense oligonucleotide, each specific to the nucleic acid encoding the protein of interest or the fragment thereof, or a combination thereof.

In another specific embodiment, the compound capable of specifically binding to the nucleic acid encoding the protein of interest or the fragment thereof may include the polynucleotide capable of specifically binding to the nucleic acid encoding the protein of interest or the fragment thereof, and may be CRISPR-Cas including guide RNA specific to the nucleic acid encoding the protein of interest or the fragment thereof. In a specific embodiment, the Cas may be Cas9.

Clustered Regularly Interspaced Short Palindromic Repeats (CRISPRs) mean loci including many short direct repeats found in the genome of bacteria or archaea, of which genetic sequences are revealed. The CRISPR-Cas system includes Cas9 as an essential protein element which forms a complex with guide RNA (specifically, two RNAs, called CRISPR RNA (crRNA) and trans-activating crRNA (tracrRNA), included in guide RNA), and it serves as an active endonuclease.

In a specific embodiment, for the CRISPR-Cas system to specifically act on the target gene of interest, the guide RNA may have a form of a dual RNA including CRISPR RNA (crRNA) and transactivating crRNA (tracrRNA) specific to the nucleic acid encoding the protein of interest, or a single strand guide RNA including parts of the crRNA and the tracrRNA and hybridizing with the nucleic acid encoding the protein of interest. The dual RNA and the single strand guide RNA may at least partially hybridize with the polynucleotide encoding the protein of interest.

Specifically, the guide RNA may be a dual RNA including crRNA and tracrRNA that hybridize with a target sequence selected from the nucleotide sequence encoding the protein of interest, or a single strand guide RNA including parts of the crRNA and the tracrRNA and hybridizing with the nucleotide encoding the protein of interest. The gene of interest which is the target sequence includes a polynucleotide sequence at least partially complementary to the crRNA or sgRNA, and a sequence including a protospacer-adjacent motif (PAM). The PAM may be a sequence well-known in the art, which may have a sequence suitable to be recognized by a nuclease protein. The gene of interest targeted by the CRISPR-Cas system may be endogenous DNA or artificial DNA. The nucleotide encoding the protein of interest may be specifically endogenous DNA of a eukaryotic cell, and more specifically, endogenous DNA of a chondrocyte.

In a specific embodiment, the crRNA or sgRNA may include twenty consecutive polynucleotides complementary to the target DNA. A nucleic acid encoding the Cas9 protein or the Cas9 protein may be derived from a microorganism of the genus Streptococcus. The microorganism of the genus Streptococcus may be Streptococcus pyogenes. The PAM may mean 5′-NGG-3′ trinucledotide, and the Cas9 protein may further include a nuclear localization signal (NLS) at the C-terminus or N-terminus to enhance the efficiency.

In the composition for promoting production of bone from eukaryotic cells of the present disclosure, the eukaryotic cells may be yeast cells, fungal cells, protozoa cells, plant cells, higher plant cells, insect cells, amphibian cells, or mammalian cells. The mammal may vary such as humans, monkeys, cows, horses, pigs, etc. The eukaryotic cells may include cultured cells (in vitro) isolated from an individual, graft cells, in vivo cells, or recombinant cells, but are not limited thereto. The eukaryotic cells isolated from an individual may be eukaryotic cells isolated from an individual the same as an individual into which the product including bone produced from the eukaryotic cells is injected. In this case, it is advantageous in that side effects such as unnecessary hyperimmune reactions or rejection reactions including graft-versus-host reaction generated by injecting a product produced from a different individual may be prevented.

In a specific embodiment, the eukaryotic cells may be fibroblasts or chondrocytes or mesenchymal stem cells or osteoprogenitor cells (MC3T3-E1; preosteoblasts).

### MAST4

MAST4 is a protein derived from a human (Homo sapiens) or a mouse (Musmusculus), but the same protein may also be expressed in other mammals such as monkeys, cows, horses, etc.

The human-derived MAST4 may include any of twelve isoforms present in human cells. The twelve isoforms may include amino acid sequences as below. The isoform sequences are based on NCBI reference sequence.

```
Isoform 1-NP_055998.1
(SEQ ID NO: 1)
      1 MDESSILRRR GLQKELSLPR RGSLIDSQKW NCLVKRCRIS NRKSLIGNGQ SPALPRPHSP
    61 LSAHAGNSPQ DSPRNFSPSA SAHFSFARRT DGRRWSLASL PSSGYGINTP SSTVSSSCSS
  121 QEKLHQLPYQ PTPDELHFLS KHFCTTESIA TENRCRNTPM RPRSRSLSPG RSPACCDHEI
  181 IMMNHVYKER FPKATAQMEE RLKEIITSYS PDNVLPLADG VLSFTHHQII ELARDCLDKS
  241 HQGLITSRYF LELQHKLDKL LQEAHDRSES GELAFIKQLV RKILIVIARP ARLLECLEFD
  301 PEEFYYLLEA AEGHAKEGQG IKTDIPRYII SQLGLNKDPL EEMAHLGNYD SGTAETPETD
  361 ESVSSSNASL KLRRKPRESD FETIKLISNG AYGAVYFVRH KESRQRFAMK KINKQNLILR
  421 NQIQQAFVER DILTFAENPF VVSMYCSFET RRHLCMVMEY VEGGDCATLM KNMGPLPVDM
  481 ARMYFAETVL ALEYLHNYGI VHRDLKPDNL LVTSMGHIKL TDFGLSKVGL MSMTTNLYEG
  541 HIEKDAREFL DKQVCGTPEY IAPEVILRQG YGKPVDWWAM GIILYEFLVG CVPFFGDTPE
  601 ELFGQVISDE INWPEKDEAP PPDAQDLITL LLRQNPLERL GTGGAYEVKQ HRFFRSLDWN
  661 SLLRQKAEFI PQLESEDDTS YFDTRSEKYH HMETEEEDDT NDEDENVEIR QFSSCSHRFS
  721 KVFSSIDRIT QNSAEEKEDS VDKTKSTTLP STETLSWSSE YSEMQQLSTS NSSDTESNRH
  781 KLSSGLLPKL AISTEGEQDE AASCPGDPHE EPGKPALPPE ECAQEEPEVT TPASTISSST
  841 LSVGSFSEHL DQINGRSECV DSTDNSSKPS SEPASHMARQ RLESTEKKKI SGKVTKSLSA
  901 SALSLMIPGD MFAVSPLGSP MSPHSLSSDP SSSRDSSPSR DSSAASASPH QPIVIHSSGK
  961 NYGFTIRAIR VYVGDSDIYT VHHIVWNVEE GSPACQAGLK AGDLITHING EPVHGLVHTE
1021 VIELLLKSGN KVSITTTPFE NISIKIGPAR RNSYKSRMVR RSKKSKKKES LERRRSLFKK
1081 LAKQPSPLLH TSRSFSCLNR SLSSGESLPG SPTHSLSPRS PTPSYRSTPD FPSGINSSQS
1141 SSPSSSAPNS PAGSGHIRPS TLHGLAPKLG GQRYRSGRRK SAGNIPLSPL ARTPSPTPQP
1201 TSPQRSPSPL LGHSLGNSKI AQAFPSKMHS PPTIVRHIVR PKSAEPPRSP LLKRVQSEEK
1261 LSPSYGSDKK HLCSRKHSLE VTQEEVQREQ SQREAPLQSL DENVCDVPPL SRARPVEQGC
1321 LKRPVSRKVG RQESVDDLDR DKLKAKVVVK KADGFPEKQE SHQKSHGPGS DLENFALFKL
1381 EEREKKVYPK AVERSSTFEN KASMQEAPPL GSLLKDALHK QASVRASEGA MSDGRVPAEH
1441 RQGGGDFRRA PAPGTLQDGL CHSLDRGISG KGEGTEKSSQ AKELLRCEKL DSKLANIDYL
1501 RKKMSLEDKE DNLCPVLKPK MTAGSHECLP GNPVRPTGGQ QEPPPASESR AFVSSTHAAQ
1561 MSAVSFVPLK ALTGRVDSGT EKPGLVAPES PVRKSPSEYK LEGRSVSCLK PIEGTLDIAL
1621 LSGPQASKTE LPSPESAQSP SPSGDVRASV PPVLPSSSGK KNDTTSAREL SPSSLKMNKS
1681 YLLEPWFLPP SRGLQNSPAV SLPDPEFKRD RKGPHPTARS PGTVMESNPQ QREGSSPKHQ
1741 DHTTDPKLLT CLGQNLHSPD LARPRCPLPP EASPSREKPG LRESSERGPP TARSERSAAR
1801 ADTCREPSME LCFPETAKTS DNSKNLLSVG RTHPDFYTQT QAMEKAWAPG GKINHKDGPG
1861 EARPPPRDNS SLHSAGIPCE KELGKVRRGV EPKPEALLAR RSLQPPGIES EKSEKLSSFP
1921 SLQKDGAKEP ERKEQPLQRH PSSIPPPPLT AKDLSSPAAR QHCSSPSHAS GREPGAKPST
1981 AEPSSSPQDP PKPVAAHSES SSHKPRPGPD PGPPKTKHPD RSLSSQKPSV GATKGKEPAT
2041 QSLGGSSREG KGHSKSGPDV FPATPGSQNK ASDGIGQGEG GPSVPLHIDR APLDAKPQPT
2101 SGGRPLEVLE KPVHLPRPGH PGPSEPADQK LSAVGEKQTL SPKHPKPSTV KDCPTLCKQT
2161 DNRQTDKSPS QPAANTDRRA EGKKCTEALY APAEGDKLEA GLSFVHSENR LKGAERPAAG
2221 VGKGFPEARG KGPGPQKPPT EADKPNGMKR SPSATGQSSF RSTALPEKSL SCSSSFPETR
2281 AGVREASAAS SDISSAKAAG GMLELPAPSN RDHRKAQPAG EGRTHMTKSD SLPSFRVSTL
2341 PLESHHPDPN TMGGASHRDR ALSVTATVGE TKGKDPAPAQ PPPARKQNVG RDVTKPSPAP
2401 NTDRPISLSN EKDFVVRQRR GKESLRSSPH KKAL,
Isoform 2-NP_942123.1
(SEQ ID NO: 2)
      1 MGEKVSEAPE PVPRGCSGHG SRTPASALVA ASSPGASSAE SSSGSETLSE EGEPGGFSRE
    61 HQPPPPPPLG GTLGARAPAA WAPASVLLER GVLALPPPLP GGAVPPAPRG SSASQEEQDE
  121 ELDHILSPPP MPFRKCSNPD VASGPGKSLK YKRQLSEDGR QLRRGSLGGA LTGRYLLPNP
  181 VAGQAWPASA ETSNLVRMRS QALGQSAPSL TASLKELSLP RRGSLIDSQK WNCLVKRPVC
  241 PNAGRTSPLG,
Isoform 3-NP_001158136.1
(SEQ ID NO: 3)
      1 MGEKVSEAPE PVPRGCSGHG SRTPASALVA ASSPGASSAE SSSGSETLSE EGEPGGFSRE
    61 HQPPPPPPLG GTLGARAPAA WAPASVLLER GVLALPPPLP GGAVPPAPRG SSASQEEQDE
  121 ELDHILSPPP MPFRKCSNPD VASGPGKSLK YKRQLSEDGR QLRRGSLGGA LTGRYLLPNP
  181 VAGQAWPASA ETSNLVRMRS QALGQSAPSL TASLKELSLP RRGSFCRISN RKSLIGNGQS
  241 PALPRPHSPL SAHAGNSPQD SPRNFSPSAS AHFSFARRTD GRRWSLASLP SSGYGTNTPS
  301 STVSSSCSSQ EKLHQLPYQP TPDELHFLSK HFCTTESIAT ENRCRNTPMR PRSRSLSPGR
  361 SPACCDHEII MMNHVYKERF PKATAQMEER LKEIITSYSP DNVLPLADGV LSFTHHQIIE
  421 LARDCLDKSH QGLITSRYFL ELQHKLDKLL QEAHDRSESG ELAFIKQLVR KILIVIARPA
  481 RLLECLEFDP EEFYYLLEAA EGHAKEGQGI KTDIPRYIIS QLGLNKDPLE EMAHLGNYDS
  541 GTAETPETDE SVSSSNASLK LRRKPRESDF ETIKLISNGA YGAVYFVRHK ESRQRFAMKK
  601 INKQNLILRN QIQQAFVERD ILTFAENPFV VSMYCSFETR RHLCMVMEYV EGGDCATLMK
  661 NMGPLPVDMA RMYFAETVLA LEYLHNYGIV HRDLKPDNLL VTSMGHIKLT DFGLSKVGLM
  721 SMTTNLYEGH IEKDAREFLD KQVCGTPEYI APEVILRQGY GKPVDWWAMG IILYEFLVGC
  781 VPFFGDTPEE LFGQVISDEI NWPEKDEAPP PDAQDLITLL LRQNPLERLG TGGAYEVKQH
  841 RFFRSLDWNS LLRQKAEFIP QLESEDDTSY FDTRSEKYHH METEEEDDIN DEDENVEIRQ
  901 FSSCSHRFSK VFSSIDRITQ NSAEEKEDSV DKTKSTILPS TETLSWSSEY SEMQQLSTSN
  961 SSDTESNRHK LSSGLLPKLA ISTEGEQDEA ASCPGDPHEE PGKPALPPEE CAQEEPEVTT
1021 PASTISSSTL SVGSFSEHLD QINGRSECVD STDNSSKPSS EPASHMARQR LESTEKKKIS
1081 GKVTKSLSAS ALSLMIPGDM FAVSPLGSPM SPHSLSSDPS SSRDSSPSRD SSAASASPHQ
1141 PIVIHSSGKN YGFTIRAIRV YVGDSDIYTV HHIVWNVEEG SPACQAGLKA GDLITHINGE
1201 PVHGLVHTEV IELLLKSGNK VSITTTPFEN TSIKTGPARR NSYKSRMVRR SKKSKKKESL
1261 ERRRSLFKKL AKQPSPLLHT SRSFSCLNRS LSSGESLPGS PTHSLSPRSP TPSYRSTPDF
1321 PSGTNSSQSS SPSSSAPNSP AGSGHIRPST LHGLAPKLGG QRYRSGRRKS AGNIPLSPLA
1381 RTPSPTPQPT SPQRSPSPLL GHSIGNSKIA QAFPSKMHSP PTIVRHIVRP KSAEPPRSPL
1441 LKRVQSEEKL SPSYGSDKKH LCSRKHSLEV TQEEVQREQS QREAPLQSLD ENVCDVPPLS
1501 RARPVEQGCL KRPVSRKVGR QESVDDLDRD KLKAKVVVKK ADGFPEKQES HQKSHGPGSD
1561 LENFALFKLE EREKKVYPKA VERSSTFENK ASMQEAPPLG SLLKDALHKQ ASVRASEGAM
1621 SDGRVPAEHR QGGGDERRAP APGTLQDGLC HSLDRGISGK GEGTEKSSQA KELLRCEKLD
1681 SKLANIDYLR KKMSLEDKED NLCPVLKPKM TAGSHECLPG NPVRPTGGQQ EPPPASESRA
1741 FVSSTHAAQM SAVSFVPLKA LTGRVDSGTE KPGLVAPESP VRKSPSEYKL EGRSVSCLKP
1801 IEGTLDIALL SGPQASKTEL PSPESAQSPS PSGDVRASVP PVLPSSSGKK NDTTSARELS
1861 PSSLKMNKSY LLEPWFLPPS RGLQNSPAVS LPDPEFKRDR KGPHPTARSP GTVMESNPQQ
1921 REGSSPKHQD HTTDPKLLTC LGQNLHSPDL ARPRCPLPPE ASPSREKPGL RESSERGPPT
1981 ARSERSAARA DTCREPSMEL CFPETAKTSD NSKNLLSVGR THPDFYTQTQ AMEKAWAPGG
2041 KINHKDGPGE ARPPPRDNSS LHSAGIPCEK ELGKVRRGVE PKPEALLARR SLQPPGIESE
2101 KSEKLSSFPS LQKDGAKEPE RKEQPLQRHP SSIPPPPLTA KDLSSPAARQ HCSSPSHASG
2161 REPGAKPSTA EPSSSPQDPP KPVAAHSESS SHKPRPGPDP GPPKTKHPDR SLSSQKPSVG
2221 ATKGKEPATQ SLGGSSREGK GHSKSGPDVF PATPGSQNKA SDGIGQGEGG PSVPLHTDRA
2281 PLDAKPQPTS GGRPLEVLEK PVHLPRPGHP GPSEPADQKL SAVGEKQTLS PKHPKPSTVK
2341 DCPTLCKQTD NRQTDKSPSQ PAANTDRRAE GKKCTEALYA PAEGDKLEAG LSFVHSENRL
2401 KGAERPAAGV GKGFPEARGK GPGPQKPPTE ADKPNGMKRS PSATGQSSFR STALPEKSLS
2461 CSSSFPETRA GVREASAASS DTSSAKAAGG MLELPAPSNR DHRKAQPAGE GRTHMTKSDS
2521 LPSFRVSILP LESHHPDPNT MGGASHRDRA LSVTATVGET KGKDPAPAQP PPARKQNVGR
2581 DVTKPSPAPN TDRPISLSNE KDFVVRQRRG KESLRSSPHK KAL,
Isoform 4-NP_001277155.1
(SEQ ID NO: 4)
      1 MKAQRERLQI PGLTLDCRTS NRKSLIGNGQ SPALPRPHSP LSAHAGNSPQ DSPRNFSPSA
    61 SAHFSFARRN DRIDGRRWSL ASLPSSGYGT NTPSSTVSSS CSSQEKLHQL PYQPTPDELH
  121 FLSKHFCTTE SIATENRCRN TPMRPRSRSL SPGRSPACCD HEIIMMNHVY KERFPKATAQ
  181 MEERLKEIIT SYSPDNVLPL ADGVLSFTHH QIIELARDCL DKSHQGLITS RYFLELQHKL
  241 DKLLQEAHDR SESGELAFIK QLVRKILIVI ARPARLLECL EFDPEEFYYL LEAAEGHAKE
  301 GQGIKTDIPR YIISQLGLNK DPLEEMAHLG NYDSGTAETP ETDESVSSSN ASLKLRRKPR
  361 ESDFETIKLI SNGAYGAVYF VRHKESRQRF AMKKINKQNL ILRNQIQQAF VERDILTFAE
  421 NPFVVSMYCS FETRRHLCMV MEYVEGGDCA TLMKNMGPLP VDMARMYFAE TVLALEYLHN
  481 YGIVHRDLKP DNLLVISMGH IKLIDFGLSK VGLMSMITNL YEGHIEKDAR EFLDKQVCGT
  541 PEYIAPEVIL RQGYGKPVDW WAMGIILYEF LVGCVPFFGD TPEELFGQVI SDEINWPEKD
  601 EAPPPDAQDL ITLLLRQNPL ERLGTGGAYE VKQHRFFRSL DWNSLLRQKA EFIPQLESED
  661 DTSYFDTRSE KYHHMETEEE DDINDEDFNV EIRQFSSCSH RFSKVFSSID RITQNSAEEK
  721 EDSVDKTKST TLPSTETLSW SSEYSEMQQL STSNSSDTES NRHKLSSGLL PKLAISTEGE
  781 QDEAASCPGD PHEEPGKPAL PPEECAQEEP EVITPASTIS SSTLSVGSFS EHLDQINGRS
  841 ECVDSTDNSS KPSSEPASHM ARQRLESTEK KKISGKVIKS LSASALSLMI PGDMFAVSPL
  901 GSPMSPHSLS SDPSSSRDSS PSRDSSAASA SPHQPIVIHS SGKNYGETIR AIRVYVGDSD
  961 IYTVHHIVWN VEEGSPACQA GLKAGDLITH INGEPVHGLV HTEVIELLLK SGNKVSITTT
1021 PFENTSIKTG PARRNSYKSR MVRRSKKSKK KESLERRRSL FKKLAKQPSP LLHTSRSFSC
1081 LNRSLSSGES LPGSPTHSLS PRSPTPSYRS TPDFPSGINS SQSSSPSSSA PNSPAGSGHI
1141 RPSTLHGLAP KLGGQRYRSG RRKSAGNIPL SPLARTPSPT PQPTSPQRSP SPLLGHSLGN
1201 SKIAQAFPSK MHSPPTIVRH IVRPKSAEPP RSPLLKRVQS EEKLSPSYGS DKKHLCSRKH
1261 SLEVTQEEVQ REQSQREAPL QSLDENVCDV PPLSRARPVE QGCLKRPVSR KVGRQESVDD
1321 LDRDKLKAKV VVKKADGFPE KQESHQKSHG PGSDLENFAL FKLEEREKKV YPKAVERSST
1381 FENKASMQEA PPLGSLLKDA LHKQASVRAS EGAMSDGRVP AEHRQGGGDF RRAPAPGTLQ
1441 DGLCHSLDRG ISGKGEGTEK SSQAKELLRC EKLDSKLANI DYLRKKMSLE DKEDNLCPVL
1501 KPKMTAGSHE CLPGNPVRPT GGQQEPPPAS ESRAFVSSTH AAQMSAVSFV PLKALTGRVD
1561 SGTEKPGLVA PESPVRKSPS EYKLEGRSVS CLKPIEGTLD IALLSGPQAS KTELPSPESA
1621 QSPSPSGDVR ASVPPVLPSS SGKKNDTTSA RELSPSSLKM NKSYLLEPWF LPPSRGLQNS
1681 PAVSLPDPEF KRDRKGPHPT ARSPGTVMES NPQQREGSSP KHQDHTTDPK LLTCLGQNLH
1741 SPDLARPRCP LPPEASPSRE KPGLRESSER GPPTARSERS AARADTCREP SMELCFPETA
1801 KTSDNSKNLL SVGRTHPDFY TQTQAMEKAW APGGKINHKD GPGEARPPPR DNSSLHSAGI
1861 PCEKELGKVR RGVEPKPEAL LARRSLQPPG IESEKSEKLS SFPSLQKDGA KEPERKEQPL
1921 QRHPSSIPPP PLTAKDLSSP AARQHCSSPS HASGREPGAK PSTAEPSSSP QDPPKPVAAH
1981 SESSSHKPRP GPDPGPPKTK HPDRSLSSQK PSVGATKGKE PATQSLGGSS REGKGHSKSG
2041 PDVFPATPGS QNKASDGIGQ GEGGPSVPLH TDRAPLDAKP QPTSGGRPLE VLEKPVHLPR
2101 PGHPGPSEPA DQKLSAVGEK QTLSPKHPKP STVKDCPTLC KQTDNRQTDK SPSQPAANTD
2161 RRAEGKKCTE ALYAPAEGDK LEAGLSFVHS ENRLKGAERP AAGVGKGFPE ARGKGPGPQK
2221 PPTEADKPNG MKRSPSATGQ SSFRSTALPE KSLSCSSSFP ETRAGVREAS AASSDTSSAK
2281 AAGGMLELPA PSNRDHRKAQ PAGEGRTHMT KSDSLPSFRV STLPLESHHP DPNTMGGASH
2341 RDRALSVTAT VGETKGKDPA PAQPPPARKQ NVGRDVTKPS PAPNTDRPIS LSNEKDFVVR
2401 QRRGKESLRS SPHKKAL,
Isoform 5-NP_001277156.1
(SEQ ID NO: 5)
      1 MDMSDPNEWT VLSNFTLPHL RSGNRLRRTQ SCRISNRKSL IGNGQSPALP RPHSPLSAHA
    61 GNSPQDSPRN FSPSASAHFS FARRTDGRRW SLASLPSSGY GTNTPSSTVS SSCSSQEKLH
  121 QLPYQPTPDE LHFLSKHFCT TESIATENRC RNTPMRPRSR SLSPGRSPAC CDHEIIMMNH
  181 VYKERFPKAT AQMEERLKEI ITSYSPDNVL PLADGVLSFT HHQIIELARD CLDKSHQGLI
  241 TSRYFLELQH KLDKLLQEAH DRSESGELAF IKQLVRKILI VIARPARLLE CLEFDPEEFY
  301 YLLEAAEGHA KEGQGIKTDI PRYIISQLGL NKDPLEEMAH LGNYDSGTAE TPETDESVSS
  361 SNASLKLRRK PRESDFETIK LISNGAYGAV YFVRHKESRQ RFAMKKINKQ NLILRNQIQQ
  421 AFVERDILTF AENPFVVSMY CSFETRRHLC MVMEYVEGGD CATLMKNMGP LPVDMARMYF
  481 AETVLALEYL HNYGIVHRDL KPDNLLVTSM GHIKLIDFGL SKVGLMSMTT NLYEGHIEKD
  541 AREFLDKQVC GTPEYIAPEV ILRQGYGKPV DWWAMGIILY EFLVGCVPFF GDTPEELFGQ
  601 VISDEINWPE KDEAPPPDAQ DLITLLLRQN PLERLGTGGA YEVKQHRFFR SLDWNSLLRQ
  661 KAEFIPQLES EDDTSYFDTR SEKYHHMETE EEDDINDEDF NVEIRQFSSC SHRFSKVESS
  721 IDRITQNSAE EKEDSVDKIK STTLPSTETL SWSSEYSEMQ QLSTSNSSDT ESNRHKLSSG
  781 LLPKLAISTE GEQDEAASCP GDPHEEPGKP ALPPEECAQE EPEVITPAST ISSSTLSDMF
  841 AVSPLGSPMS PHSLSSDPSS SRDSSPSRDS SAASASPHQP IVIHSSGKNY GFTIRAIRVY
  901 VGDSDIYTVH HIVWNVEEGS PACQAGLKAG DLITHINGEP VHGLVHTEVI ELLLKSGNKV
  961 SITTTPFENT SIKTGPARRN SYKSRMVRRS KKSKKKESLE RRRSLFKKLA KQPSPLLHTS
1021 RSFSCLNRSL SSGESLPGSP THSLSPRSPT PSYRSTPDFP SGTNSSQSSS PSSSAPNSPA
1081 GSGHIRPSTL HGLAPKLGGQ RYRSGRRKSA GNIPLSPLAR TPSPTPQPTS PQRSPSPLLG
1141 HSLGNSKIAQ AFPSKMHSPP TIVRHIVRPK SAEPPRSPLL KRVQSEEKLS PSYGSDKKHL
1201 CSRKHSLEVT QEEVQREQSQ REAPLQSLDE NVCDVPPLSR ARPVEQGCLK RPVSRKVGRQ
1261 ESVDDLDRDK LKAKVVVKKA DGFPEKQESH QKSHGPGSDL ENFALFKLEE REKKVYPKAV
1321 ERSSTFENKA SMQEAPPLGS LLKDALHKQA SVRASEGAMS DGRVPAEHRQ GGGDERRAPA
1381 PGTLQDGLCH SLDRGISGKG EGTEKSSQAK ELLRCEKLDS KLANIDYLRK KMSLEDKEDN
1441 LCPVLKPKMT AGSHECLPGN PVRPTGGQQE PPPASESRAF VSSTHAAQMS AVSFVPLKAL
1501 TGRVDSGTEK PGLVAPESPV RKSPSEYKLE GRSVSCLKPI EGILDIALLS GPQASKTELP
1561 SPESAQSPSP SGDVRASVPP VLPSSSGKKN DTTSARELSP SSLKMNKSYL LEPWFLPPSR
1621 GLQNSPAVSL PDPEFKRDRK GPHPTARSPG TVMESNPQQR EGSSPKHQDH TTDPKLLTCL
1681 GQNLHSPDLA RPRCPLPPEA SPSREKPGLR ESSERGPPTA RSERSAARAD TCREPSMELC
1741 FPETAKTSDN SKNLLSVGRT HPDFYTQTQA MEKAWAPGGK TNHKDGPGEA RPPPRDNSSL
1801 HSAGIPCEKE LGKVRRGVEP KPEALLARRS LQPPGIESEK SEKLSSFPSL QKDGAKEPER
1861 KEQPLQRHPS SIPPPPLTAK DLSSPAARQH CSSPSHASGR EPGAKPSTAE PSSSPQDPPK
1921 PVAAHSESSS HKPRPGPDPG PPKTKHPDRS LSSQKPSVGA TKGKEPATQS LGGSSREGKG
1981 HSKSGPDVFP ATPGSQNKAS DGIGQGEGGP SVPLHTDRAP LDAKPQPTSG GRPLEVLEKP
2041 VHLPRPGHPG PSEPADQKLS AVGEKQTLSP KHPKPSTVKD CPTLCKQTDN RQTDKSPSQP
2101 AANTDRRAEG KKCTEALYAP AEGDKLEAGL SFVHSENRLK GAERPAAGVG KGFPEARGKG
2161 PGPQKPPTEA DKPNGMKRSP SATGQSSFRS TALPEKSLSC SSSFPETRAG VREASAASSD
2221 TSSAKAAGGM LELPAPSNRD HRKAQPAGEG RTHMTKSDSL PSFRVSTLPL ESHHPDPNTM
2281 GGASHRDRAL SVTATVGETK GKDPAPAQPP PARKQNVGRD VTKPSPAPNT DRPISLSNEK
2341 DFVVRQRRGK ESLRSSPHKK AL,
Isoform 6-NP_001277157.1
(SEQ ID NO: 6)
      1 MGEKVSEAPE PVPRGCSGHG SRTPASALVA ASSPGASSAE SSSGSETLSE EGEPGGFSRE
    61 HQPPPPPPLG GTLGARAPAA WAPASVLLER GVLALPPPLP GGAVPPAPRG SSASQEEQDE
  121 ELDHILSPPP MPFRKCSNPD VASGPGKSLK YKRQLSEDGR QLRRGSLGGA LTGRYLLPNP
  181 VAGQAWPASA ETSNLVRMRS QALGQSAPSL TASLKELSLP RRGSFPVCPN AGRISPLG,
Isoform 7-NP_001284580.1
(SEQ ID NO: 7)
      1 MDMSDPNFWT VLSNFTLPHL RSGNRLRRTQ SCRISNRKSL IGNGQSPALP RPHSPLSAHA
    61 GNSPQDSPRN FSPSASAHFS FARRTDGRRW SLASLPSSGY GTNTPSSTVS SSCSSQEKLH
  121 QLPYQPTPDE LHFLSKHFCT TESIATENRC RNTPMRPRSR SLSPGRSPAC CDHEIIMMNH
  181 VYKERFPKAT AQMEERLKEI ITSYSPDNVL PLADGVLSFT HHQIIELARD CLDKSHQGLI
  241 TSRYFLELQH KLDKLLQEAH DRSESGELAF IKQLVRKILI VIARPARLLE CLEFDPEEFY
  301 YLLEAAEGHA KEGQGIKTDI PRYIISQLGL NKDPLEEMAH LGNYDSGTAE TPETDESVSS
  361 SNASLKLRRK PRESDFETIK LISNGAYGAV YFVRHKESRQ RFAMKKINKQ NLILRNQIQQ
  421 AFVERDILIF AENPFVVSMY CSFETRRHLC MVMEYVEGGD CATLMKNMGP LPVDMARMYF
  481 AETVLALEYL HNYGIVHRDL KPDNLLVTSM GHIKLIDFGL SKVGLMSMTT NLYEGHIEKD
  541 AREFLDKQVC GTPEYIAPEV ILRQGYGKPV DWWAMGIILY EFLVGCVPFF GDTPEELFGQ
  601 VISDEINWPE KDEAPPPDAQ DLITLLLRQN PLERLGTGGA YEVKQHRFFR SLDWNSLLRQ
  661 KAEFIPQLES EDDTSYFDTR SEKYHHMETE EEDDINDEDF NVEIRQFSSC SHRFSKVFSS
  721 IDRITQNSAE EKEDSVDKTK STTLPSTETL SWSSEYSEMQ QLSTSNSSDT ESNRHKLSSG
  781 LLPKLAISTE GEQDEAASCP GDPHEEPGKP ALPPEECAQE EPEVTTPAST ISSSTLSVGS
  841 FSEHLDQING RSECVDSTDN SSKPSSEPAS HMARQRLEST EKKKISGKVT KSLSASALSL
  901 MIPGDMFAVS PLGSPMSPHS LSSDPSSSRD SSPSRDSSAA SASPHQPIVI HSSGKNYGFT
  961 IRAIRVYVGD SDIYTVHHIV WNVEEGSPAC QAGLKAGDLI THINGEPVHG LVHTEVIELL
1021 LKSGNKVSIT TTPFENTSIK TGPARRNSYK SRMVRRSKKS KKKESLERRR SLFKKLAKQP
1081 SPLLHTSRSF SCLNRSLSSG ESLPGSPTHS LSPRSPTPSY RSTPDFPSGT NSSQSSSPSS
1141 SAPNSPAGSG HIRPSTLHGL APKLGGQRYR SGRRKSAGNI PLSPLARTPS PTPQPTSPQR
1201 SPSPLLGHSL GNSKIAQAFP SKMHSPPTIV RHIVRPKSAE PPRSPLLKRV QSEEKLSPSY
1261 GSDKKHLCSR KHSLEVTQEE VQREQSQREA PLQSLDENVC DVPPLSRARP VEQGCLKRPV
1321 SRKVGRQESV DDLDRDKLKA KVVVKKADGF PEKQESHQKS HGPGSDLENF ALFKLEEREK
1381 KVYPKAVERS STFENKASMQ EAPPLGSLLK DALHKQASVR ASEGAMSDGR VPAEHRQGGG
1441 DFRRAPAPGT LQDGLCHSLD RGISGKGEGT EKSSQAKELL RCEKLDSKLA NIDYLRKKMS
1501 LEDKEDNLCP VLKPKMTAGS HECLPGNPVR PTGGQQEPPP ASESRAFVSS THAAQMSAVS
1561 FVPLKALTGR VDSGTEKPGL VAPESPVRKS PSEYKLEGRS VSCLKPIEGT LDIALLSGPQ
1621 ASKTELPSPE SAQSPSPSGD VRASVPPVLP SSSGKKNDTT SARELSPSSL KMNKSYLLEP
1681 WFLPPSRGLQ NSPAVSLPDP EFKRDRKGPH PTARSPGTVM ESNPQQREGS SPKHQDHTTD
1741 PKLLTCLGQN LHSPDLARPR CPLPPEASPS REKPGLRESS ERGPPTARSE RSAARADTCR
1801 EPSMELCFPE TAKTSDNSKN LLSVGRTHPD FYTQTQAMEK AWAPGGKINH KDGPGEARPP
1861 PRDNSSLHSA GIPCEKELGK VRRGVEPKPE ALLARRSLQP PGIESEKSEK LSSFPSLQKD
1921 GAKEPERKEQ PLQRHPSSIP PPPLTAKDLS SPAARQHCSS PSHASGREPG AKPSTAEPSS
1981 SPQDPPKPVA AHSESSSHKP RPGPDPGPPK TKHPDRSLSS QKPSVGATKG KEPATQSLGG
2041 SSREGKGHSK SGPDVFPATP GSQNKASDGI GQGEGGPSVP LHTDRAPLDA KPQPTSGGRP
2101 LEVLEKPVHL PRPGHPGPSE PADQKLSAVG EKQTLSPKHP KPSTVKDCPT LCKQTDNRQT
2161 DKSPSQPAAN TDRRAEGKKC TEALYAPAEG DKLEAGLSFV HSENRLKGAE RPAAGVGKGF
2221 PEARGKGPGP QKPPTEADKP NGMKRSPSAT GQSSFRSTAL PEKSLSCSSS FPETRAGVRE
2281 ASAASSDISS AKAAGGMLEL PAPSNRDHRK AQPAGEGRTH MTKSDSLPSF RVSTLPLESH
2341 HPDPNTMGGA SHRDRALSVT ATVGETKGKD PAPAQPPPAR KQNVGRDVTK PSPAPNTDRP
2401 ISLSNEKDFV VRQRRGKESL RSSPHKKAL,
Isoform 8-NP_001380453.1
(SEQ ID NO: 8)
      1 MGEKVSEAPE PVPRGCSGHG SRTPASALVA ASSPGASSAE SSSGSETLSE EGEPGGFSRE
    61 HQPPPPPPLG GTLGARAPAA WAPASVLLER GVLALPPPLP GGAVPPAPRG SSASQEEQDE
  121 ELDHILSPPP MPFRKCSNPD VASGPGKSLK YKRQLSEDGR QLRRGSLGGA LTGRYLLPNP
  181 VAGQAWPASA ETSNLVRMRS QALGQSAPSL TASLKELSLP RRGSFCRTSN RKSLIGNGQS
  241 PALPRPHSPL SAHAGNSPQD SPRNFSPSAS AHFSFARRND RIDGRRWSLA SLPSSGYGTN
  301 TPSSTVSSSC SSQEKLHQLP YQPTPDELHF LSKHFCTTES IATENRCRNT PMRPRSRSLS
  361 PGRSPACCDH EIIMMNHVYK ERFPKATAQM EERLKEIITS YSPDNVLPLA DGVLSFTHHQ
  421 IIELARDCLD KSHQGLITSR YFLELQHKLD KLLQEAHDRS ESGELAFIKQ LVRKILIVIA
  481 RPARLLECLE FDPEEFYYLL EAAEGHAKEG QGIKTDIPRY IISQLGLNKD PLEEMAHLGN
  541 YDSGTAETPE TDESVSSSNA SLKLRRKPRE SDFETIKLIS NGAYGAVYFV RHKESRQRFA
  601 MKKINKQNLI LRNQIQQAFV ERDILTFAEN PFVVSMYCSF ETRRHLCMVM EYVEGGDCAT
  661 LMKNMGPLPV DMARMYFAET VLALEYLHNY GIVHRDLKPD NLLVTSMGHI KLTDFGLSKV
  721 GLMSMTTNLY EGHIEKDARE FLDKQVCGTP EYIAPEVILR QGYGKPVDWW AMGIILYEFL
  781 VGCVPFFGDT PEELFGQVIS DEINWPEKDE APPPDAQDLI TLLLRQNPLE RLGTGGAYEV
  841 KQHRFFRSLD WNSLLRQKAE FIPQLESEDD TSYFDTRSEK YHHMETEEED DINDEDENVE
  901 IRQFSSCSHR FSKVFSSIDR ITQNSAEEKE DSVDKTKSTT LPSTETLSWS SEYSEMQQLS
  961 TSNSSDTESN RHKLSSGLLP KLAISTEGEQ DEAASCPGDP HEEPGKPALP PEECAQEEPE
1021 VTTPASTISS STLSVGSFSE HLDQINGRSE CVDSTDNSSK PSSEPASHMA RQRLESTEKK
1081 KISGKVIKSL SASALSLMIP GDMFAVSPLG SPMSPHSLSS DPSSSRDSSP SRDSSAASAS
1141 PHQPIVIHSS GKNYGFTIRA IRVYVGDSDI YTVHHIVWNV EEGSPACQAG LKAGDLITHI
1201 NGEPVHGLVH TEVIELLLKS GNKVSITTTP FENTSIKTGP ARRNSYKSRM VRRSKKSKKK
1261 ESLERRRSLF KKLAKQPSPL LHTSRSFSCL NRSLSSGESL PGSPTHSLSP RSPTPSYRST
1321 PDFPSGINSS QSSSPSSSAP NSPAGSGHIR PSTLHGLAPK LGGQRYRSGR RKSAGNIPLS
1381 PLARTPSPTP QPTSPQRSPS PLLGHSIGNS KIAQAFPSKM HSPPTIVRHI VRPKSAEPPR
1441 SPLLKRVQSE EKLSPSYGSD KKHLCSRKHS LEVTQEEVQR EQSQREAPLQ SLDENVCDVP
1501 PLSRARPVEQ GCLKRPVSRK VGRQESVDDL DRDKLKAKVV VKKADGFPEK QESHQKSHGP
1561 GSDLENFALF KLEEREKKVY PKAVERSSTF ENKASMQEAP PLGSLLKDAL HKQASVRASE
1621 GAMSDGRVPA EHRQGGGDFR RAPAPGTLQD GLCHSLDRGI SGKGEGTEKS SQAKELLRCE
1681 KLDSKLANID YLRKKMSLED KEDNLCPVLK PKMTAGSHEC LPGNPVRPTG GQQEPPPASE
1741 SRAFVSSTHA AQMSAVSFVP LKALTGRVDS GTEKPGLVAP ESPVRKSPSE YKLEGRSVSC
1801 LKPIEGTLDI ALLSGPQASK TELPSPESAQ SPSPSGDVRA SVPPVLPSSS GKKNDTTSAR
1861 ELSPSSLKMN KSYLLEPWFL PPSRGLQNSP AVSLPDPEFK RDRKGPHPTA RSPGTVMESN
1921 PQQREGSSPK HQDHTTDPKL LTCLGQNLHS PDLARPRCPL PPEASPSREK PGLRESSERG
1981 PPTARSERSA ARADTCREPS MELCFPETAK TSDNSKNLLS VGRTHPDFYT QTQAMEKAWA
2041 PGGKINHKDG PGEARPPPRD NSSLHSAGIP CEKELGKVRR GVEPKPEALL ARRSLQPPGI
2101 ESEKSEKLSS FPSLQKDGAK EPERKEQPLQ RHPSSIPPPP LTAKDLSSPA ARQHCSSPSH
2161 ASGREPGAKP STAEPSSSPQ DPPKPVAAHS ESSSHKPRPG PDPGPPKTKH PDRSLSSQKP
2221 SVGATKGKEP ATQSLGGSSR EGKGHSKSGP DVFPATPGSQ NKASDGIGQG EGGPSVPLHT
2281 DRAPLDAKPQ PTSGGRPLEV LEKPVHLPRP GHPGPSEPAD QKLSAVGEKQ TLSPKHPKPS
2341 TVKDCPTLCK QTDNRQTDKS PSQPAANTDR RAEGKKCTEA LYAPAEGDKL EAGLSFVHSE
2401 NRLKGAERPA AGVGKGFPEA RGKGPGPQKP PTEADKPNGM KRSPSATGQS SFRSTALPEK
2461 SLSCSSSFPE TRAGVREASA ASSDTSSAKA AGGMLELPAP SNRDHRKAQP AGEGRTHMTK
2521 SDSLPSFRVS TLPLESHHPD PNTMGGASHR DRALSVTATV GETKGKDPAP AQPPPARKQN
2581 VGRDVTKPSP APNTDRPISL SNEKDFVVRQ RRGKESLRSS PHKKAL,
Isoform 9-NP_001380454.1
(SEQ ID NO: 9)
      1 MGEKVSEAPE PVPRGCSGHG SRTPASALVA ASSPGASSAE SSSGSETLSE EGEPGGFSRE
    61 HQPPPPPPLG GTLGARAPAA WAPASVLLER GVLALPPPLP GGAVPPAPRG SSASQEEQDE
  121 ELDHILSPPP MPFRKCSNPD VASGPGKSLK YKRQLSEDGR QLRRGSLGGA LTGRYLLPNP
  181 VAGQAWPASA ETSNLVRMRS QALGQSAPSL TASLKELSLP RRGSFCRTSN RKSLIGNGQS
  241 PALPRPHSPL SAHAGNSPQD SPRNFSPSAS AHFSFARRTD GRRWSLASLP SSGYGTNTPS
  301 STVSSSCSSQ EKLHQLPYQP TPDELHFLSK HFCTTESIAT ENRCRNTPMR PRSRSLSPGR
  361 SPACCDHEII MMNHVYKERF PKATAQMEER LKEIITSYSP DNVLPLADGV LSFTHHQIIE
  421 LARDCLDKSH QGLITSRYFL ELQHKLDKLL QEAHDRSESG ELAFIKQLVR KILIVIARPA
  481 RLLECLEFDP EEFYYLLEAA EGHAKEGQGI KTDIPRYIIS QLGLNKDPLE EMAHLGNYDS
  541 GTAETPETDE SVSSSNASLK LRRKPRESDF ETIKLISNGA YGAVYFVRHK ESRQRFAMKK
  601 INKQNLILRN QIQQAFVERD ILTFAENPFV VSMYCSFETR RHLCMVMEYV EGGDCATLMK
  661 NMGPLPVDMA RMYFAETVLA LEYLHNYGIV HRDLKPDNLL VTSMGHIKLT DFGLSKVGLM
  721 SMTTNLYEGH IEKDAREFLD KQVCGTPEYI APEVILRQGY GKPVDWWAMG IILYEFLVGC
  781 VPFFGDTPEE LFGQVISDEI NWPEKDEAPP PDAQDLITLL LRQNPLERLG TGGAYEVKQH
  841 RFFRSLDWNS LLRQKAEFIP QLESEDDTSY FDTRSEKYHH METEEEDDIN DEDENVEIRQ
  901 FSSCSHRFSK VFSSIDRITQ NSAEEKEDSV DKIKSTILPS TETLSWSSEY SEMQQLSTSN
  961 SSDTESNRHK LSSGLLPKLA ISTEGEQDEA ASCPGDPHEE PGKPALPPEE CAQEEPEVTT
1021 PASTISSSTL SDMFAVSPLG SPMSPHSLSS DPSSSRDSSP SRDSSAASAS PHQPIVIHSS
1081 GKNYGFTIRA IRVYVGDSDI YTVHHIVWNV EEGSPACQAG LKAGDLITHI NGEPVHGLVH
1141 TEVIELLLKS GNKVSITTTP FENTSIKTGP ARRNSYKSRM VRRSKKSKKK ESLERRRSLF
1201 KKLAKQPSPL LHTSRSFSCL NRSLSSGESL PGSPTHSLSP RSPTPSYRST PDFPSGINSS
1261 QSSSPSSSAP NSPAGSGHIR PSTLHGLAPK LGGQRYRSGR RKSAGNIPLS PLARIPSPTP
1321 QPTSPQRSPS PLLGHSIGNS KIAQAFPSKM HSPPTIVRHI VRPKSAEPPR SPLLKRVQSE
1381 EKLSPSYGSD KKHLCSRKHS LEVTQEEVQR EQSQREAPLQ SLDENVCDVP PLSRARPVEQ
1441 GCLKRPVSRK VGRQESVDDL DRDKLKAKVV VKKADGFPEK QESHQKSHGP GSDLENFALF
1501 KLEEREKKVY PKAVERSSTF ENKASMQEAP PLGSLLKDAL HKQASVRASE GAMSDGRVPA
1561 EHRQGGGDFR RAPAPGTLQD GLCHSLDRGI SGKGEGTEKS SQAKELLRCE KLDSKLANID
1621 YLRKKMSLED KEDNLCPVLK PKMTAGSHEC LPGNPVRPTG GQQEPPPASE SRAFVSSTHA
1681 AQMSAVSFVP LKALTGRVDS GTEKPGLVAP ESPVRKSPSE YKLEGRSVSC LKPIEGTLDI
1741 ALLSGPQASK TELPSPESAQ SPSPSGDVRA SVPPVLPSSS GKKNDITSAR ELSPSSLKMN
1801 KSYLLEPWFL PPSRGLQNSP AVSLPDPEFK RDRKGPHPTA RSPGTVMESN PQQREGSSPK
1861 HQDHTTDPKL LTCLGQNLHS PDLARPRCPL PPEASPSREK PGLRESSERG PPTARSERSA
1921 ARADTCREPS MELCFPETAK TSDNSKNLLS VGRTHPDFYT QTQAMEKAWA PGGKINHKDG
1981 PGEARPPPRD NSSLHSAGIP CEKELGKVRR GVEPKPEALL ARRSLQPPGI ESEKSEKLSS
2041 FPSLQKDGAK EPERKEQPLQ RHPSSIPPPP LTAKDLSSPA ARQHCSSPSH ASGREPGAKP
2101 STAEPSSSPQ DPPKPVAAHS ESSSHKPRPG PDPGPPKTKH PDRSLSSQKP SVGATKGKEP
2161 ATQSLGGSSR EGKGHSKSGP DVFPATPGSQ NKASDGIGQG EGGPSVPLHT DRAPLDAKPQ
2221 PTSGGRPLEV LEKPVHLPRP GHPGPSEPAD QKLSAVGEKQ TLSPKHPKPS TVKDCPTLCK
2281 QTDNRQTDKS PSQPAANTDR RAEGKKCTEA LYAPAEGDKL EAGLSFVHSE NRLKGAERPA
2341 AGVGKGFPEA RGKGPGPQKP PTEADKPNGM KRSPSATGQS SFRSTALPEK SLSCSSSFPE
2401 TRAGVREASA ASSDTSSAKA AGGMLELPAP SNRDHRKAQP AGEGRTHMTK SDSLPSFRVS
2461 TLPLESHHPD PNTMGGASHR DRALSVTATV GETKGKDPAP AQPPPARKQN VGRDVTKPSP
2521 APNTDRPISL SNEKDFVVRQ RRGKESLRSS PHKKAL,
Isoform 10-NP_001380455.1
(SEQ ID NO: 10)
      1 MDESSILRRR GLQKELSLPR RGSLIDSQKW NCLVKRCRIS NRKSLIGNGQ SPALPRPHSP
    61 LSAHAGNSPQ DSPRNFSPSA SAHFSFARRT DGRRWSLASL PSSGYGINTP SSTVSSSCSS
  121 QEKLHQLPYQ PTPDELHFLS KHFCTTESIA TENRCRNTPM RPRSRSLSPG RSPACCDHEI
  181 IMMNHVYKER FPKATAQMEE RLKEIITSYS PDNVLPLADG VLSFTHHQII ELARDCLDKS
  241 HQGLITSRYF LELQHKLDKL LQEAHDRSES GELAFIKQLV RKILIVIARP ARLLECLEFD
  301 PEEFYYLLEA AEGHAKEGQG IKTDIPRYII SQLGLNKDPL EEMAHLGNYD SGTAETPETD
  361 ESVSSSNASL KLRRKPRESD FETIKLISNG AYGAVYFVRH KESRQRFAMK KINKQNLILR
  421 NQIQQAFVER DILTFAENPF VVSMYCSFET RRHLCMVMEY VEGGDCATLM KNMGPLPVDM
  481 ARMYFAETVL ALEYLHNYGI VHRDLKPDNL LVTSMGHIKL TDFGLSKVGL MSMTINLYEG
  541 HIEKDAREFL DKQVCGTPEY IAPEVILRQG YGKPVDWWAM GIILYEFLVG CVPFFGDTPE
  601 ELFGQVISDE INWPEKDEAP PPDAQDLITL LLRQNPLERL GIGGAYEVKQ HRFFRSLDWN
  661 SLLRQKAEFI PQLESEDDIS YFDTRSEKYH HMETEEEDDT NDEDENVEIR QFSSCSHRFS
  721 KVFSSIDRIT QNSAEEKEDS VDKTKSTTLP STETLSWSSE YSEMQQLSTS NSSDTESNRH
  781 KLSSGLLPKL AISTEGEQDE AASCPGDPHE EPGKPALPPE ECAQEEPEVT TPASTISSST
  841 LSDMFAVSPL GSPMSPHSLS SDPSSSRDSS PSRDSSAASA SPHQPIVIHS SGKNYGETIR
  901 AIRVYVGDSD IYTVHHIVWN VEEGSPACQA GLKAGDLITH INGEPVHGLV HTEVIELLLK
  961 SGNKVSITTT PFENTSIKTG PARRNSYKSR MVRRSKKSKK KESLERRRSL FKKLAKQPSP
1021 LLHTSRSFSC LNRSLSSGES LPGSPTHSLS PRSPTPSYRS TPDFPSGINS SQSSSPSSSA
1081 PNSPAGSGHI RPSTLHGLAP KLGGQRYRSG RRKSAGNIPL SPLARTPSPT PQPTSPQRSP
1141 SPLLGHSLGN SKIAQAFPSK MHSPPTIVRH IVRPKSAEPP RSPLLKRVQS EEKLSPSYGS
1201 DKKHLCSRKH SLEVTQEEVQ REQSQREAPL QSLDENVCDV PPLSRARPVE QGCLKRPVSR
1261 KVGRQESVDD LDRDKLKAKV VVKKADGFPE KQESHQKSHG PGSDLENFAL FKLEEREKKV
1321 YPKAVERSST FENKASMQEA PPLGSLLKDA LHKQASVRAS EGAMSDGRVP AEHRQGGGDF
1381 RRAPAPGTLQ DGLCHSLDRG ISGKGEGTEK SSQAKELLRC EKLDSKLANI DYLRKKMSLE
1441 DKEDNLCPVL KPKMTAGSHE CLPGNPVRPT GGQQEPPPAS ESRAFVSSTH AAQMSAVSFV
1501 PLKALTGRVD SGTEKPGLVA PESPVRKSPS EYKLEGRSVS CLKPIEGTLD IALLSGPQAS
1561 KTELPSPESA QSPSPSGDVR ASVPPVLPSS SGKKNDTTSA RELSPSSLKM NKSYLLEPWF
1621 LPPSRGLQNS PAVSLPDPEF KRDRKGPHPT ARSPGTVMES NPQQREGSSP KHQDHTTDPK
1681 LLTCLGQNLH SPDLARPRCP LPPEASPSRE KPGLRESSER GPPTARSERS AARADTCREP
1741 SMELCFPETA KTSDNSKNLL SVGRTHPDFY TQTQAMEKAW APGGKINHKD GPGEARPPPR
1801 DNSSLHSAGI PCEKELGKVR RGVEPKPEAL LARRSLQPPG IESEKSEKLS SFPSLQKDGA
1861 KEPERKEQPL QRHPSSIPPP PLTAKDLSSP AARQHCSSPS HASGREPGAK PSTAEPSSSP
1921 QDPPKPVAAH SESSSHKPRP GPDPGPPKTK HPDRSLSSQK PSVGATKGKE PATQSLGGSS
1981 REGKGHSKSG PDVFPATPGS QNKASDGIGQ GEGGPSVPLH TDRAPLDAKP QPTSGGRPLE
2041 VLEKPVHLPR PGHPGPSEPA DQKLSAVGEK QTLSPKHPKP STVKDCPTLC KQTDNRQTDK
2101 SPSQPAANTD RRAEGKKCTE ALYAPAEGDK LEAGLSFVHS ENRLKGAERP AAGVGKGFPE
2161 ARGKGPGPQK PPTEADKPNG MKRSPSATGQ SSFRSTALPE KSLSCSSSFP ETRAGVREAS
2221 AASSDTSSAK AAGGMLELPA PSNRDHRKAQ PAGEGRTHMT KSDSLPSFRV STLPLESHHP
2281 DPNTMGGASH RDRALSVTAT VGETKGKDPA PAQPPPARKQ NVGRDVTKPS PAPNTDRPIS
2341 LSNEKDFVVR QRRGKESLRS SPHKKAL,
Isoform 11-NP_001380456.1
(SEQ ID NO: 11)
      1 MDMSDPNFWT VLSNFTLPHL RSGNRLRRTQ SCRISNRKSL IGNGQSPALP RPHSPLSAHA
    61 GNSPQDSPRN FSPSASAHFS FARRIDGRRW SLASLPSSGY GTNTPSSTVS SSCSSQEKLH
  121 QLPYQPTPDE LHFLSKHFCT TESIATENRC RNTPMRPRSR SLSPGRSPAC CDHEIIMMNH
  181 VYKERFPKAT AQMEERLKEI ITSYSPDNVL PLADGVLSFT HHQIIELARD CLDKSHQGLI
  241 TSRYFLELQH KLDKLLQEAH DRSESGELAF IKQLVRKILI VIARPARLLE CLEFDPEEFY
  301 YLLEAAEGHA KEGQGIKTDI PRYIISQLGL NKDPLEEMAH LGNYDSGTAE TPETDESVSS
  361 SNASLKLRRK PRESDFETIK LISNGAYGAV YFVRHKESRQ RFAMKKINKQ NLILRNQIQQ
  421 AFVERDILIF AENPFVVSMY CSFETRRHLC MVMEYVEGGD CATLMKNMGP LPVDMARMYF
  481 AETVLALEYL HNYGIVHRDL KPDNLLVTSM GHIKLIDFGL SKVGLMSMTT NLYEGHIEKD
  541 AREFLDKQVC GTPEYIAPEV ILRQGYGKPV DWWAMGIILY EFLVGCVPFF GDTPEELFGQ
  601 VISDEINWPE KDEAPPPDAQ DLITLLLRQN PLERLGTGGA YEVKQHRFFR SLDWNSLLRQ
  661 KAEFIPQLES EDDTSYFDTR SEKYHHMETE EEDDINDEDF NVEIRQFSSC SHRFSKVESS
  721 IDRITQNSAE EKEDSVDKIK STILPSTETL SWSSEYSEMQ QLSTSNSSDT ESNRHKLSSG
  781 LLPKLAISTE GEQDEAASCP GDPHEEPGKP ALPPEECAQE EPEVTTPAST ISSSTLSDMF
  841 AVSPLGSPMS PHSLSSDPSS SRDSSPSRDS SAASASPHQP IVIHSSGKNY GFTIRAIRVY
  901 VGDSDIYTVH HIVWNVEEGS PACQAGLKAG DLITHINGEP VHGLVHTEVI ELLLKSGNKV
  961 SITTTPFENT SIKTGPARRN SYKSRMVRRS KKSKKKESLE RRRSLFKKLA KQPSPLLHTS
1021 RSFSCLNRSL SSGESLPGSP THSLSPRSPT PSYRSTPDFP SGINSSQSSS PSSSAPNSPA
1081 GSGHIRPSTL HGLAPKLGGQ RYRSGRRKSA GNIPLSPLAR TPSPTPQPTS PQRSPSPLLG
1141 HSLGNSKIAQ AFPSKMHSPP TIVRHIVRPK SAEPPRSPLL KRVQSEEKLS PSYGSDKKHL
1201 CSRKHSLEVT QEEVQREQSQ REAPLQSLDE NVCDVPPLSR ARPVEQGCLK RPVSRKVGRQ
1261 ESVDDLDRDK LKAKVVVKKA DGFPEKQESH QKSHGPGSDL ENFALFKLEE REKKVYPKAV
1321 ERSSTFENKA SMQEAPPLGS LLKDALHKQA SVRASEGAMS DGRVPAEHRQ GGGDERRAPA
1381 PGTLQDGLCH SLDRGISGKG EGTEKSSQAK ELLRCEKLDS KLANIDYLRK KMSLEDKEDN
1441 LCPVLKPKMT AGSHECLPGN PVRPTGGQQE PPPASESRAF VSSTHAAQMS AVSFVPLKAL
1501 TGRVDSGTEK PGLVAPESPV RKSPSEYKLE GRSVSCLKPI EGILDIALLS GPQASKTELP
1561 SPESAQSPSP SGDVRASVPP VLPSSSGKKN DTTSARELSP SSLKMNKSYL LEPWFLPPSR
1621 GLQNSPAVSL PDPEFKRDRK GPHPTARSPG TVMESNPQQR EGSSPKHQDH TTDPKLLTCL
1681 GQNLHSPDLA RPRCPLPPEA SPSREKPGLR ESSERGPPTA RSERSAARAD TCREPSMELC
1741 FPETAKTSDN SKNLLSVGRT HPDFYTQTQA MEKAWAPGGK TNHKDGPGEA RPPPRDNSSL
1801 HSAGIPCEKE LGKVRRGVEP KPEALLARRS LQPPGIESEK SEKLSSFPSL QKDGAKEPER
1861 KEQPLQRHPS SIPPPPLTAK DLSSPAARQH CSSPSHASGR EPGAKPSTAE PSSSPQDPPK
1921 PVAAHSESSS HKPRPGPDPG PPKTKHPDRS LSSQKPSVGA TKGKEPATQS LGGSSREGKG
1981 HSKSGPDVFP ATPGSQNKAS DGIGQGEGGP SVPLHTDRAP LDAKPQPTSG GRPLEVLEKP
2041 VHLPRPGHPG PSEPADQKLS AVGEKQTLSP KHPKPSTVKD CPTLCKQTDN RQTDKSPSQP
2101 AANTDRRAEG KKCTEALYAP AEGDKLEAGL SFVHSENRLK GAERPAAGVG KGFPEARGKG
2161 PGPQKPPTEA DKPNGMKRSP SATGQSSFRS TALPEKSLSC SSSFPETRAG VREASAASSD
2221 TSSAKAAGGM LELPAPSNRD HRKAQPAGEG RTHMTKSDSL PSFRVSTLPL ESHHPDPNTM
2281 GGASHRDRAL SVTATVGETK GKDPAPAQPP PARKQNVGRD VTKPSPAPNT DRPISLSNEK
2341 DFVVRQRRGK ESLRSSPHKK AL,
or
Isoform 12-NP_001380457.1
(SEQ ID NO: 12)
      1 MDESSILRRR GLQKELSLPR RGSFCRISNR KSLIGNGQSP ALPRPHSPLS AHAGNSPQDS
    61 PRNFSPSASA HFSFARRIDG RRWSLASLPS SGYGTNTPSS TVSSSCSSQE KLHQLPYQPT
  121 PDELHFLSKH FCTTESIATE NRCRNTPMRP RSRSLSPGRS PACCDHEIIM MNHVYKERFP
  181 KATAQMEERL KEIITSYSPD NVLPLADGVL SFTHHQIIEL ARDCLDKSHQ GLITSRYFLE
  241 LQHKLDKLLQ EAHDRSESGE LAFIKQLVRK ILIVIARPAR LLECLEFDPE EFYYLLEAAE
  301 GHAKEGQGIK TDIPRYIISQ LGLNKDPLEE MAHLGNYDSG TAETPETDES VSSSNASLKL
  361 RRKPRESDFE TIKLISNGAY GAVYFVRHKE SRQRFAMKKI NKQNLILRNQ IQQAFVERDI
  421 LTFAENPFVV SMYCSFETRR HLCMVMEYVE GGDCATLMKN MGPLPVDMAR MYFAETVLAL
  481 EYLHNYGIVH RDLKPDNLLV TSMGHIKLID FGLSKVGLMS MTTNLYEGHI EKDAREFLDK
  541 QVCGTPEYIA PEVILRQGYG KPVDWWAMGI ILYEFLVGCV PFFGDTPEEL FGQVISDEIN
  601 WPEKDEAPPP DAQDLITLLL RQNPLERLGT GGAYEVKQHR FFRSLDWNSL LRQKAEFIPQ
  661 LESEDDTSYF DTRSEKYHHM ETEEEDDIND EDENVEIRQF SSCSHRFSKV FSSIDRITQN
  721 SAEEKEDSVD KTKSTTLPST ETLSWSSEYS EMQQLSTSNS SDTESNRHKL SSGLLPKLAI
  781 STEGEQDEAA SCPGDPHEEP GKPALPPEEC AQEEPEVTTP ASTISSSTLS DMFAVSPLGS
  841 PMSPHSLSSD PSSSRDSSPS RDSSAASASP HQPIVIHSSG KNYGFTIRAI RVYVGDSDIY
  901 TVHHIVWNVE EGSPACQAGL KAGDLITHIN GEPVHGLVHT EVIELLLKSG NKVSITTTPF
  961 ENTSIKTGPA RRNSYKSRMV RRSKKSKKKE SLERRRSLFK KLAKQPSPLL HTSRSFSCLN
1021 RSLSSGESLP GSPTHSLSPR SPTPSYRSTP DFPSGINSSQ SSSPSSSAPN SPAGSGHIRP
1081 STLHGLAPKL GGQRYRSGRR KSAGNIPLSP LARTPSPTPQ PTSPQRSPSP LLGHSLGNSK
1141 IAQAFPSKMH SPPTIVRHIV RPKSAEPPRS PLLKRVQSEE KLSPSYGSDK KHLCSRKHSL
1201 EVTQEEVQRE QSQREAPLQS LDENVCDVPP LSRARPVEQG CLKRPVSRKV GRQESVDDLD
1261 RDKLKAKVVV KKADGFPEKQ ESHQKSHGPG SDLENFALFK LEEREKKVYP KAVERSSTFE
1321 NKASMQEAPP LGSLLKDALH KQASVRASEG AMSDGRVPAE HRQGGGDERR APAPGTLQDG
1381 LCHSLDRGIS GKGEGTEKSS QAKELLRCEK LDSKLANIDY LRKKMSLEDK EDNLCPVLKP
1441 KMTAGSHECL PGNPVRPIGG QQEPPPASES RAFVSSTHAA QMSAVSFVPL KALTGRVDSG
1501 TEKPGLVAPE SPVRKSPSEY KLEGRSVSCL KPIEGTLDIA LLSGPQASKT ELPSPESAQS
1561 PSPSGDVRAS VPPVLPSSSG KKNDTTSARE LSPSSLKMNK SYLLEPWFLP PSRGLQNSPA
1621 VSLPDPEFKR DRKGPHPTAR SPGTVMESNP QQREGSSPKH QDHTTDPKLL TCLGQNLHSP
1681 DLARPRCPLP PEASPSREKP GLRESSERGP PTARSERSAA RADICREPSM ELCFPETAKT
1741 SDNSKNLLSV GRTHPDFYTQ TQAMEKAWAP GGKTNHKDGP GEARPPPRDN SSLHSAGIPC
1801 EKELGKVRRG VEPKPEALLA RRSLQPPGIE SEKSEKLSSF PSLQKDGAKE PERKEQPLQR
1861 HPSSIPPPPL TAKDLSSPAA RQHCSSPSHA SGREPGAKPS TAEPSSSPQD PPKPVAAHSE
1921 SSSHKPRPGP DPGPPKIKHP DRSLSSQKPS VGATKGKEPA TQSLGGSSRE GKGHSKSGPD
1981 VFPATPGSQN KASDGIGQGE GGPSVPLHTD RAPLDAKPQP TSGGRPLEVL EKPVHLPRPG
2041 HPGPSEPADQ KLSAVGEKQT LSPKHPKPST VKDCPTLCKQ TDNRQTDKSP SQPAANTDRR
2101 AEGKKCTEAL YAPAEGDKLE AGLSFVHSEN RLKGAERPAA GVGKGFPEAR GKGPGPQKPP
2161 TEADKPNGMK RSPSATGQSS FRSTALPEKS LSCSSSFPET RAGVREASAA SSDISSAKAA
2221 GGMLELPAPS NRDHRKAQPA GEGRTHMTKS DSLPSFRVST LPLESHHPDP NTMGGASHRD
2281 RALSVTATVG ETKGKDPAPA QPPPARKQNV GRDVTKPSPA PNTDRPISLS NEKDFVVRQR
2341 RGKESLRSSP HKKAL
```

An amino acid sequence or a polynucleotide sequence having biologically equivalent activity, even though it is not identical to the amino acid sequences of SEQ ID NOS: 1 to 12 may also be regarded as the MAST4 protein or mRNA thereof.

Therefore, in a specific embodiment, the MAST4 protein may include any one sequence of SEQ ID NOS: 1 to 12 and the nucleotide sequence encoding the MAST4 protein.

The MAST4 protein or polypeptide may include an amino acid sequence having 60% or more, for example, 70% or more, 80% or more, 90% or more, 95% or more, 99% or more, or 100% sequence identity to SEQ ID NOS: 1 to 12. Further, the MAST4 protein may have an amino acid sequence having modification of 1 or more amino acids, 2 or more amino acids, 3 or more amino acids, 4 or more amino acids, 5 or more amino acids, 6 or more amino acids, or 7 or more amino acids in the amino acid sequences of SEQ ID NOS: 1 to 12.

Each polynucleotide encoding MAST4 may have a sequence having 60% or more, for example, 70% or more, 80% or more, 90% or more, 95% or more, 99% or more, or 100% sequence identity to the sequence encoding any of the MAST4 protein. Further, the polynucleotide encoding MAST4 may be a polynucleotide having a different sequence of 1 or more nucleotides, 2 or more nucleotides, 3 or more nucleotides, 4 or more nucleotides, 5 or more nucleotides, 6 or more nucleotides, or 7 or more nucleotides in the sequences encoding SEQ ID NOS: 1-12.

The present inventors first demonstrated that production of bone is increased by increasing expression of or stabilizing MAST4 gene expression in eucaryotic cells such as mesenchymal stem cells or osteoprogenitor cells.

Further, in a specific embodiment, the composition for promoting the production of extracellular matrix from the eukaryotic cells may be used for tissue regeneration or anti-aging.

The term “pharmaceutically acceptable salt” means any organic or inorganic addition salt of the compound in the composition of the present disclosure, whose concentration has effective action because it is relatively non-toxic and harmless to patients and whose side effects do not degrade the beneficial efficacy of the composition of the present disclosure. These salts may be selected from any one known to those skilled in the art.

The composition of the present disclosure may further include a pharmaceutically acceptable carrier. The composition including the pharmaceutically acceptable carrier may have various formulations for parenteral administration. When formulated, the composition may be prepared using commonly used diluents or excipients such as fillers, extenders, binders, wetting agents, disintegrating agents, surfactants, etc.

Formulations for parenteral administration may include sterilized aqueous solutions, non-aqueous solvents, suspensions, emulsions, freeze-dried preparations, suppositories, etc. The non-aqueous solvents and suspensions may include propylene glycol, polyethylene glycol, vegetable oils such as olive oil, injectable esters such as ethyl oleate, etc. As a base of a suppository, witepsol, macrogol, Tween 61, cocoa butter, laurin butter, glycerol, gelatin, etc. may be used.

As used herein, “stabilizing MAST4” means preventing degradation or preventing inhibition of activity of MAST4. For example, MAST4 may be destabilized or degraded by being ubiquitinated and subject to proteolysis through proteasome. Thus, an inhibitor of an agent that targets MAST4 for degradation is contemplated in the invention.

### Inhibitor of GSK-3

Glycogen synthase kinase-3 (GSK-3) is a proline-directed serine-threonine kinase. There are two isoforms, GSK-3α and β, that are highly related and largely redundant. Their many substrates range from regulators of cellular metabolism to molecules that control growth and differentiation. A sampling of inhibitors of GSK-3beta include but not limited to, Laduviglusib (CHIR-99021) HCl (CAS No. 1797989-42-4), SB216763 (CAS No. 280744 Sep. 4), AT7519 (CAS No. 844442-38-2), CHIR-98014 (CAS No. 252935-94-7), TWS119 (CAS No. 601514-19-6) are some of the chemical compounds that may be used to inhibit GSK-3beta activity. See Selleck Chemicals, Houston, TX (2023). Antibodies also exist that specifically inhibit GSK-3beta or GSK-3alpha.

### Therapy for Bone Generation

The present invention discloses ex vivo technique involving culturing of eucaryotic cells, in which a protein that inhibits production or activity of MAST4 is inhibited from being expressed or inhibited post-translationally, followed by transplantation of the modified eucaryotic cells to the target bone defect area of the mammalian host so as to effect generation of bone. Alternatively, or simultaneously, MAST4 expression is caused to be increased in the cell.

It will be understood by the artisan of ordinary skill that the preferred source of cells for treating a human patient is the patient's own connective tissue cells or mesenchymal stem cells, such as autologous fibroblast or osteoprogenitor cells (bone progenitor cells), osteocytes, preosteoblasts, osteoblasts or osteoclasts, but that allogeneic cells may be also used.

More specifically, this method may include using an inhibitor to GSK-3, including GSK-3alpha or GSK-3beta.

Another embodiment of this invention provides for a compound for parenteral administration to a patient in a prophylactically effective amount that includes the modified cells and a suitable pharmaceutical carrier.

In the present application, a method is provided for generating or regenerating bone by injecting an appropriate mammalian cell that is transfected or transduced with a gene encoding MAST4, which is overexpressed.

In an embodiment of the invention, it is understood that the cells may be injected into the area in which bone is to be generated or regenerated with or without scaffolding material or any other auxiliary material, such as extraneous cells or other biocompatible carriers.

The method of the present invention may be applied to all types of bones in the body, including but not limited to, non-union fractures (fractures that fail to heal), craniofacial reconstruction, segmental defect due to tumor removal, augmentation of bone around a hip implant revision (i.e., 25% of hip implants are replacements of an existing implant, as the lifespan of a hip implant is only ˜10 years), reconstruction of bone in the jaw for dental purposes. Other target bones include vertebrae on the spine for spine fusion, large bones, and so on. Further, the present invention may be used to treat fracture or defect in femur, tibia, hip, hip joint fracture especially in the elderly and so forth by administering the inventive cell to the subject in need thereof.

The cells to be modified include any appropriate mammalian cells including mesenchymal stem cells, and connective tissue cell, which assists in the formation of bone, including, but not limited to, fibroblast cells, osteoprogenitor cells, preosteoblasts, osteoblasts, osteocytes and osteoclasts, and may further include chondrocytes. However, it is understood that other non-genetically modified cells may also be included in the composition that is used to contact the bone defect site, such as preosteoblasts, osteoblasts, osteocytes, osteoclasts, chondrocytes, and so on.

Where mention is made of “bone defect” or “defected bone”, it is to be understood that such defects may include fractures, breaks, and/or degradation of the bone including such conditions caused by injuries or diseases, and further may include defects in the spine vertebrae and further degradation of the disc area between the vertebrae. In one aspect of the invention, pain caused by the degradation of disk space between vertebrae may be treated by fusing vertebrae that surround the disk space that has degenerated.

One ex vivo method of treating a fractured or defected bone disclosed throughout this specification comprises initially generating a recombinant viral or plasmid vector which contains a DNA sequence encoding a protein or biologically active fragment thereof. This recombinant vector is then used to infect or transfect a population of in vitro cultured cells, resulting in a population of cells containing the vector. These cells are then transplanted to a target bone defected area of a mammalian host, effecting subsequent expression of the protein or protein fragment within the defected area. Expression of this DNA sequence of interest is useful in substantially repairing the fracture or defect.

More specifically, this method includes employing gene encoding MAST4, or a biologically active derivative or fragment thereof.

Another embodiment of this invention provides a method for introducing at least one gene encoding a product into at least one cell for use in treating the mammalian host. This method includes employing viral or non-viral means for introducing the gene coding for the product into the cell. More specifically, this method includes liposome encapsulation, calcium phosphate coprecipitation, electroporation, or DEAE-dextran mediation, and includes employing as the gene a gene capable of encoding a member of MAST4 family or biologically active derivative or fragment thereof, and a selectable marker, or biologically active derivative or fragment thereof.

Another embodiment of this invention provides an additional method for introducing at least one gene encoding a product into at least one cell for use in treating the mammalian host. This additional method includes employing the biologic means of utilizing a virus to deliver the DNA vector molecule to the target cell or tissue. Preferably, the virus is a pseudo-virus, the genome having been altered such that the pseudovirus is capable only of delivery and stable maintenance within the target cell, preferably not retaining an ability to replicate within the target cell or tissue. The altered viral genome is further manipulated by recombinant DNA techniques such that the viral genome acts as a DNA vector molecule which contains the heterologous gene of interest to be expressed within the target cell or tissue.

A preferred embodiment of the invention is a method of delivering a cell expressing MAST4 protein to a target defect area by delivering the MAST4 gene to the tissue of a mammalian host through use of an adeno-associated viral vector or lentiviral vector with the ex vivo technique disclosed within this specification. In other words, a DNA sequence of interest encoding a functional MAST4 protein or protein fragment is subcloned into a viral vector of choice, the recombinant viral vector is then grown to adequate titer and used to infect in vitro cultured cells, and the transduced cells, are transplanted into the bone defect region or a therapeutically effective nearby area.

Another preferred method of the present invention involves direct in vivo delivery of MAST4 gene to the connective tissue of a mammalian host through use of either an adenovirus vector, adeno-associated virus (AAV) vector or herpes-simplex virus (HSV) vector. In other words, a DNA sequence of interest encoding a functional MAST4 protein or protein fragment is subcloned into the respective viral vector. The MAST4 containing viral vector is then grown to adequate titer and directed into bone defect region or an osteogenically effective nearby area.

### Therapy for Healing Osteoporotic Bone Fracture

Osteoporosis is a structural deterioration of the skeleton caused by loss of bone mass resulting from an imbalance in bone formation, bone resorption, or both, such that resorption dominates the bone formation phase, thereby reducing the weight-bearing capacity of the affected bone. In a healthy adult, the rate at which bone is formed and resorbed is tightly coordinated so as to maintain the renewal of skeletal bone. However, in osteoporotic individuals an imbalance in these bone remodeling cycles develops which results in both loss of bone mass and in formation of microarchitectural defects in the continuity of the skeleton. These skeletal defects, created by perturbation in the remodeling sequence, accumulate and finally reach a point at which the structural integrity of the skeleton is severely compromised and bone fracture is likely. Although this imbalance occurs gradually in most individuals as they age (“senile osteoporosis”), it is much more severe and occurs at a rapid rate in postmenopausal women. In addition, osteoporosis also may result from nutritional and endocrine imbalance, hereditary disorders and a number of malignant transformations.

### Method of Making Cartilage or Bone

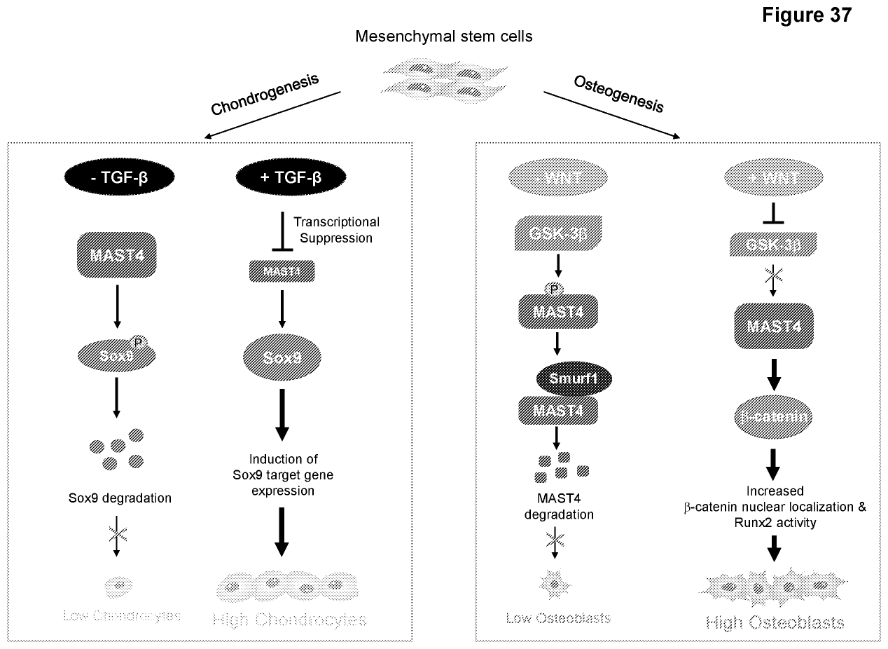

Although MSCs are intensively researched with the aim to be used in regenerative therapy, the molecular mechanisms governing the differentiation of MSCs are not fully understood. Our results suggest that Mast4 is a key molecule that determines the commitment and differentiation of MSCs towards a chondrogenic or osteogenic cell fate. We have demonstrated that TGF-β1-mediated suppression of Mast4 gene transcription leads to the increase of Sox9 protein and Smad3-Sox9 association, which results in increased Sox9 transcriptional activity, ultimately initiating MSCs to favor chondrogenesis at the expense of bone formation. In regard to osteogenesis, we have shown that Wnt-mediated inhibition of Mast4 protein degradation by inhibiting GSK-3β activity leads to the increase of β-catenin protein and Runx2 transcriptional activity, ultimately initiating MSCs to favor osteogenesis (FIG. 37).

The context-dependent nature of TGF-β has been delineated throughout the decades. Particularly, the cytostatic effect of TGF-β has shown to be orchestrated by transcriptional activation of CDK inhibitors and repression of c-Myc, highlighting its roles in the treatment of cancers³¹. Numerous studies have also identified the function of TGF-β in determining the fate of multipotent stem cells during developmental processes. In regard to endochondral ossification during skeletal development, TGF-β promotes mesenchymal condensation and chondrogenesis, but inhibits chondrocyte maturation and differentiation into osteocytes, indicating its sequential regulation along specific lineages³². The bi-functionality of TGF-β signal during skeletal development are supported by observations in animal models^{15,33}. We observed suppression of Mast4 by TGF-β during chondrogenesis in vitro and predominant expression of Mast4 in hypertrophic chondrocytes in vivo. These observations speculate that TGF-β exerts diverse regulatory influences on skeletogenesis through specific regulation of Mast4 at different stages. Moreover, our observation of Mast4 exerting no influence on the cytostatic effect of TGF-β provides compelling evidence in favor of Mast4 being a critical mediator of the TGF-β-induced chondrogenic differentiation of MSCs.

Various transcription co-factors serve as Smad partners aiding in target gene recognition and transcriptional regulation³⁴. Regarding Smad-mediated gene repression, Smad3 inhibits Runx2 activity through direct interaction, ultimately diminishing osteoblast differentiation³⁵. E2F4/5 has been demonstrated as a co-repressor in TGF-β-induced repression of c-Myc³⁶. TGF-β-induced SpB repression is associated with Smad3 interaction with Nkx2.1³⁷. Interestingly, the expected binding sites of E2F4 and Nkx2.1 near the Smad3-binding site were recognized through analysis of the Mast4 promoter region. Thus, it would also be important to investigate whether these co-transcription factors are involved in TGF-B/Smad3-mediated Mast4 regulation. In addition, discovery of novel co-transcription factors that regulate Mast4 expression along sequential stages of chondro-/osteogenic differentiation would benefit the understanding of cartilage and bone development and their regulation.

Post-translational modifications (PTMs) modulate protein functions and stability, and fine tune signal transduction³⁸. Here, we demonstrated the impacts of PTMs on the TGF-β1-Mast4-Sox9 axis during chondrogenesis. A number of signaling pathways and PTMs have been exhibited to regulate Sox9, a master transcription factor during chondrocyte differentiation, by controlling a repertoire of cartilage-related ECM genes at the early stage^{10,16,39,40}. Our observations illustrate that Mast4 promotes Sox9 degradation by inducing Sox9 phosphorylation at serine 494. Even though it remains to be elucidated whether Mast4-induced Sox9 phosphorylation is recognized by any of the E3 ligases for subsequent Sox9 and Mast4 degradation, our study shows that Mast4 is likely to be an important factor in controlling Sox9 activity. E6-AP/UBEA is an E3 ligase that induces ubiquitin-mediated proteasomal degradation of Sox9 in hypertrophic chondrocytes during endochondral ossification⁴¹. Considering that Mast4 is predominantly expressed in hypertrophic chondrocytes, it may be worth examining whether Mast4 co-operates with E6-AP/UBEA to regulate Sox9 stability in hypertrophic chondrocytes. Moreover, examination of the regulation of Mast4 on Sox9 stability through phosphorylation at serine 494 in vivo and subsequent chondrogenic differentiation ability may be necessary in the future study. In addition, a previous study indicated the importance of the delicate balance of Sox9 activity in MSC for proper differentiation. Our observation of chondrocyte accumulation in the most terminally differentiated hypertrophic state and their delayed exit from the growth plate, shown in the endochondral bones of Mast4^{-/-} mice, may be explained by Mast4 deficiency-mediated overexpression of Sox9 and collagen as well as reduction of Mmp9 and Mmp13.

With regard to Wnt/β-catenin, different mechanisms have been reported to explain Wnt-mediated β-catenin stabilization⁴². Notably, Wnt inhibits GSK3 activity towards β-catenin in various ways. Given that phosphorylation by GSK3 often marks the target proteins for ubiquitination and proteolysis, our findings that inhibition of Mast4 phosphorylation by GSK-3β increases the stability of Mast4 and subsequent β-catenin reinforce the action of Wnt/β-catenin signaling in MSCs selecting osteoblastic fate^{24,43}. Furthermore, it would be worth examining Mast4 protein level in GSK-3β- deficient mice and GSK-3β inhibitor-administered mice that display increased bone formation and bone mass^{21,44,45}.

Mast4 belongs to the MAST kinase family, consisting of Mast1-4 and Mast1⁴⁶. Mast1 through 4 share a similar domain organization having a kinase domain, a PDZ domain, and a domain of unknown function (DUF). Currently, little is known about the biological roles of the MAST kinase family. Several studies have demonstrated the association of Mast1, 2, and 3 with cancers^{47,48}. Besides its role as a neuroprotective mediator⁴⁹⁻⁵¹, Mast4 has been reported to undergo O-GlcNAc modification, of which global elevation is frequently observed during osteoblast differentiation⁵². In addition, it was demonstrated that Mast4 mediated FGF-2 signaling, known to play a role in bone formation, in Sertoli cells through induction of ERM phosphorylation at serine 367 residue⁵³. Meanwhile, the microtubule cytoskeletons have shown to contribute to the osteogenic differentiation of MSCs⁵⁴. Since the MAST kinase family shares a high degree of similarity in protein domains that are considered as structural and functional building blocks, it is likely that the MAST kinase family members are critical cellular mediators of a variety of signal transduction in normal and diseased states. In addition, examination of Mast4 regulation in the differentiation of MSCs into various lineages, including osteoblasts or adipocytes, may be worth further investigation.

In conclusion, we have demonstrated that Mast4 is a crucial mediator in MSC commitment towards chondro-osteogenic differentiation pathway. Our findings implicate a function of Masts4 in the limiting of Sox9 transcriptional activity to determine the fate of MSC development into cartilage or bone. Therefore, in the context of cell therapy, Mast4 will be an ideal target for potential MSC therapy.

### Therapeutic Composition

The formulation of therapeutic compounds is generally known in the art and reference can conveniently be made to Remington's Pharmaceutical Sciences, 17th ed., Mack Publishing Co., Easton, Pa., USA. For example, from about 0.05 μg to about 20 mg per kilogram of body weight per day may be administered. Dosage regime may be adjusted to provide the optimum therapeutic response. For example, several divided doses may be administered daily or the dose may be proportionally reduced as indicated by the exigencies of the therapeutic situation. The active compound may be administered in a convenient manner such as by the intravenous (where water soluble), intramuscular, subcutaneous, intra nasal, or intradermal.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, chlorobutanol, phenol, sorbic acid, themerosal and the like. In many cases, it will be preferable to include isotonic agents, for example, sugars or sodium chloride. Prolonged absorption of the injectable compositions can be brought about by the use in the composition of agents delaying absorption, for example, aluminium monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterile active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique, which yield a powder of the active ingredient plus any additional desired ingredient from a previously sterile-filtered solution thereof.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of the invention is dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims. The following examples are offered by way of illustration of the present invention, and not by way of limitation.

### EXAMPLES

All animal studies were approved by the Institutional Animal Care and Use Committee of KNOTUS Co., Ltd and Institutional Animal Care and Use Committee of Center for Phenogenomics Animal Research Facility and Woojung BSC, and performed in accordance with ethical and procedural guidelines. The laboratory mice were maintained on a 12-h light/dark cycle at room temperature (20-22° C.) with constant humidity (40±10%).

### Example 1—Generation of Mast4 Knockout Mice by CRISPR/Cas9 Technology

For generation of Mast4 knockout mice by CRISPR/Cas9-mediated gene targeting, we targeted exon 1 and exon 15 of Mast4 (RefSeq Accession number: 175171): 5′-GGAAACTCTGTCGGAGGAAG-3′ ID NO:13) (exon 1) and 5′-GGCACAAAGAGTCCCGCCAG-3′ (SEQ ID NO:14) (exon 15). We then inserted each sequence into pX330 plasmid, which carried both guide RNA and Cas9 expression units, received from Dr. Feng Zhang (Addgene, #42230)⁵⁵. We named these vectors pX330-Mast4-E1 and pX330-Mast4-E15.

The pregnant mare serum gonadotropin (5 units) and the human chorionic gonadotropin (5 units) were intraperitoneally injected at a 48 h interval into female C57BL/6J mice (Charles River Laboratories, Kanagawa, Japan), which were then mated with male C57BL/6J mice. The pX330-Mast4-E1 and pX330-Mast4-E15 (circular, 5 ng/μl each) were co-microinjected into 231 zygotes collected from the oviducts of the mated female mice. The survived 225 injected zygotes were transferred into the oviducts in pseudopregnant ICR female, and 47 newborns were obtained. We collected genomic DNA from the tails of 31 founder mice that survived.

To confirm indel mutations induced by CRISPR/Cas9, we amplified genomic region including the target sites by PCR with the primers for exon 1 target (MAST4-1 genotype F: 5′-GTAGGGACTCCACGCTCCAG-3′ (SEQ ID NO:15); MAST4-1 genotype R: 5′-CCGGACCCTAGTCTCTTCG-3′ (SEQ ID NO:16)) and for exon 15 target (MAST4-15 genotype F: 5′-GGGTTCTCTGCGAAAGTCAG-3′ (SEQ ID NO:17); MAST4-15 genotype R: 5′-ATCCCTGTGTTCCGTTTCAG-3′ (SEQ ID NO:18)). The PCR products were sequenced by using BigDye Terminator v3.1 Cycle Sequencing Kit (Thermo Fisher Scientific), MAST4-1 genotype F primer, and MAST4-15 genotype F primer. In male founder #38, we found indel mutations in both exon 1 and exon 15 without pX330 random integration. To identify the indel sequence and whether indel mutations in exon 1 and exon 15 occurred on the same chromosome (cis manner), the founder #38 was mated with wild-type female, and the indel mutations in F1 were sequenced. We obtained 17 F1 newborns, and 12 of them carried 71 bp deletion (chr13:103,333,981-103,334,051: GRCm38/mm10) in exon 1 and 3 bp deletion (chr13:102,774,360-102,774,362) in exon 15 in a cis manner.

### Example 2—CRISPR/Cas9-Mediated Deletion of the Mast4 in C3H10T1/2 and Human Bone Marrow-Derived Stem Cells

For C3H10T1/2 cells, lentiCRISPRv2 vector (Addgene, #52961) was digested with BsmBI and ligated with annealed oligonucleotide targeting Mast4 exon 1, 5′-TACCCTGCCGCTGCCGCACC-3′ (SEQ ID NO:19) (LentiCRISPRv2-Mast4 Ex1) and exon 2, 5′-AGCAACCCAGATGTGGCCTG-3′ (SEQ ID NO:20) (LentiCRISPRv2-Mast4 Ex2). To generate lentivirus, HEK293T cells were transfected with LentiCRISPRv2-Mast4 Ex1 and packaging vectors (pVSVG and psPAX2, Addgene #8454, #12260) using polyethylenimine at 70% confluency. Viral supernatant was harvested at 48 h post-transfection, filtered through 0.45-μm filters and applied to C3H10T1/2 cells. After puromycin-mediated selection, single-cell clones were grown in 96-well plates. From genomic DNA, the exon 1 and exon 2 regions of the Mast4 gene were amplified using AccuPower™ PCR premix (Bioneer). The indel mutations causing frameshift-mediated depletion of Mast4 protein were confirmed by sequencing. For human bone marrow-derived stem cells (hBMSC), we generated guide RNA (gRNA) using GeneArt™ Precision gRNA Synthesis Kit (Invitrogen) according to the manufacturer's protocol. Human bone marrow-derived stem cells at passage 5-6 were transfected with the gRNA targeting exon 5 (Forward: 5′-TAATACGACTCACTATAGAGCAACCGGAAAAGCTTAAT-3′ (SEQ ID NO:21); Reverse: 5′-TTCTAGCTCTAAAACATTAAGCTTTTCCGGTTGCT-3′ (SEQ ID NO:22)) and Cas9 protein (Toolgen) using the Neon Transfection System following the manufacturer's protocol. Since hBMSC were unable to form colonies from individual cells, the pools of edited cells were used for further chondrogenic differentiation, protein and mRNA isolation. The CRISPR/Cas9-mediated Mast4 gene knockout efficiency in hBMSC was determined by ICE knockout analysis (www.synthego.com). Mast4-depleted hBMSC obtained >70 of ICE and KO scores, which indicates indel percentage and the proportion of cells having frameshift or 21+bp indel, respectively, were used.

### Example 3—Lentiviral shRNA Production/Infections

Two different shRNAs targeting exon 15 and exon 22 (shMast4 Exon 15 F: CCGGCCCAGTTGATATGGCCAGAATCTCGAGATTCTGGCCATATCAACTGGGTTTTT G (SEQ ID NO:23), shMast4 Exon 22 F:CCGGCCGAAGTTTCTCCTGCTTAAACTCGAGTTTAAGCAGGAGAAACTTCGGTTTT TG (SEQ ID NO:24)) of Mast4 were designed, and annealed oligos were inserted into the pLKO.1 vectors. To generate shRNA lentivirus, 293T cells were transfected with pLKO-shMast4 (#1 and #2) or scrambled control pLKO-pGL2 together with lentiviral packaging plasmids, psPax2 and VSV-G. At 48 h post transfection, viral supernatants were harvested and filtrated. C3H10T1/2 cells were infected with shRNA lentivirus and polybrene (8 μg/ml) for 24 h, followed by puromycin selection (4 μg/ml).

### Example 4—Cell Culture and Chondrogenic/Osteogenic Differentiation

C3H10T1/2 cells (Clone 8, CCL-2260, ATCC), mouse bone marrow-derived mesenchymal stromal cells (mBMSC), and human embryonic kidney cell line HEK293T (CRL-3216, ATCC), were grown in Dulbecco's Modified Eagle's Medium (DMEM; LM001-05, WELGENE) containing 10% fetal bovine serum (FBS; S001-01, WELGENE) and 1% penicillin-streptomycin (P/S; LS202-02, WELGENE). ATDC5 (RCB0565, RIKEN BRC) cells were grown in DMEM/F-12 (11320033, Gibco) containing 5% FBS and 1% P/S. The hBMSC were kindly provided from SCM Lifescience (Incheon, S. Korea), where established hBMSC lines through the subfractionation culturing method⁵⁶. Briefly, human bone marrow aspirates from the iliac crest of three healthy donors after written informed consent approved by Inha University Hospital Institutional Review Board; IRB number 10-51, were mixed with isolation medium and incubated. The supernatants containing floating bone marrow cells without the cells settled down to the bottom were repeatedly transferred to new 100-mm dishes. After 10-14 days of incubation, well-separated colonies were isolated, expanded and characterized. These were grown in DMEM (low glucose; LM001-11, WELGENE) containing 10% FBS and 1% P/S. The human primary chondrocytes, which were collected by straining collagenase-treated cartilage tissues obtained from 1-year-old human female donor⁵⁷, were also kindly provided by SCM Lifescience. These were grown in DMEM (17-205-CVR, Corning) containing 10% FBS (26140-079, Gibco), 20 mM L-glutamine (25030-081, Gibco), and 10 μg/ml Gentamicin (15700-060, Thermo fisher). MC3T3-E1 cells (Subclone 4, CRL-2593, ATCC) were grown in Alpha Minimum Essential Medium (α-MEM) without ascorbic acid (LM008-53, WELGENE) containing 10% FBS and 1% P/S. All cells were cultured at 37° C. in a humidified 5% CO₂ incubator. For the micromass culture of C3H10T1/2 cells, 1×10⁵ cells in a 10 μl drop of normal growth medium were seeded onto the culture dish, followed by an 2 h attachment period. Then, BMP-2 (150 ng/ml; PeproTech)-containing medium was added to the dish, and the medium was replaced every 48-72 h. For the pellet culture of hBMSCs, 2×10⁵ cells were seeded onto a 15 ml conical tube and were grown in α-MEM containing 1% P/S, 10⁻⁷M of dexamethasone (Sigma Aldrich), 1/100 of ITS+Premix Universal Culture Supplement (Corning), 50 ng/ml of ascorbic acid (Sigma Aldrich), 10 ng/ml of TGF-β1 and TGF-β3 (R&D Systems), and 40 ng/ml of L-Proline (Sigma Aldrich) for 21 days. The medium was replaced every 48-72 h. For mBMSCs, cells were isolated from an aspirate of bone marrow harvested from the tibia marrow compartments and were cultured in DMEM containing 10% FBS for 3 h. Non-adherent cells were carefully removed, and fresh medium was resupplied. The cultured BMMSCs were differentiated to chondrocytes using the StemPro Chondrogenesis Differentiation Kit (Thermo Fisher Scientific) according to the manufacturer's instructions. For osteogenic differentiation of C3H10T1/2 cells, confluent cells were cultured in the maintenance medium supplemented with 50 μg/ml of ascorbic acid (Sigma Aldrich), 10 mM of β-glycerophosphate (Sigma Aldrich), and 200 ng/ml of BMP-2 for 10 days. The medium was replaced every 48-72 h.

### Example 5—Alcian Blue and Alkaline Phosphatase (ALP) Staining

The differentiated cells were washed with PBS twice and fixed in 4% paraformaldehyde at room temperature for 5-10 min. The chondrogenic differentiated cells were stained with alcian blue solution (1% alcian blue in 0.1M HCl, pH 1.0; Sigma Aldrich) overnight, followed by one wash with 0.1M HCl and two with PBS. The osteogenic differentiated cells were stained with 5-bromo-4-chloro-3-indolyl-phosphate/nitro-blue tetrazolium solution (BCIP/NBT; Merck) for 30 min at 37° C.

### Example 6—The 3D Spheroid Formation Assay

The 3D spheroid formation of C3H10T1/2 cells using low-binding plate was conducted as previously reported²³. Briefly, the round bottom ultra-low attachment 96-well microplate (Corning) was coated with gelatin (0.1%; Sigma Aldrich). Then, 1×10⁵ cells in 50 μl of BMP-2 (150 ng/ml)-containing medium were added to each well of the coated microplate and cultured for 8 days. The medium was replaced every 48-72 h.

### Example 7—RNA-Seq and Bioinformatics Analyses

For the sample preparation of RNA sequencing using the differentiating C3H10T1/2 cells, total 30 high-density micromass cultures obtained from three separate induction of chondrogenic differentiation (10 masses/each induction) of the wild-type and Mast4-depleted (KO #1) C3H10T1/2 cells were combined together for RNA sequencing. For the RNA sequencing using the cartilage and bone of mice, cartilage and bone was dissected as follows. After euthanizing Mast4^{+/+} mice and Mast4^{-/-} mice at PN 1 day in a CO₂ chamber, the middle part of the femur was cut. After removing the skin, all the muscles were removed with forceps. The fibula was removed after amputation at the articular cartilage of the knee and ankle joint of tibia. The epiphysis was separated from the body of tibia along the boundary of the calcified zone with a 30 G needle. The separated epiphyseal cartilage and tibia bone were placed in Trizol® (Invitrogen). The tibia was hemisectioned and chopped with a razor blade in Trizol®. Each sample was homogenized with an equal amount of 0.5 mm stainless steel beads. Then, RNA was obtained through layer separation using chloroform and precipitation using isopropanol. Since the total amount of RNA obtained from each mouse at PN 1 day was not sufficient for RNA sequencing analysis, RNAs obtained from the cartilage or bone of the tibias of Mast4^{+/+} mice and Mast4^{-/-} mice at PN 1 day were combined (n=3 per each group). RNA-Seq libraries were prepared using TruSeq RNA Sample Prep Kit according to the manufacturer's manual (Illumina, Inc., San Diego, CA) using 1 μg of the qualified RNA in each sample. After qPCR validation, libraries were subjected to paired-end sequencing with a 100 bp read length using an Illumina HiSeq 2500 platform, yielding an average of 57.7 million reads per library. The quality of raw reads was assessed with FastQC (version 0.11.9). Clean reads for each sample, in which average quality scores were greater than Q30, were aligned to the mouse reference genome GRCm38.p4mm10 using TopHat⁵⁸ with a set of gene model annotation. Gene expression was calculated as FPKM using Cufflinks. Differential expression analysis between the wild-type and Mast4-depleted samples was performed by using Cuffdiff⁵⁹ with a cutoff set at P<0.05 and ≥1.5-fold change in reference to qPCR validation of Sox9-targeted genes. Gene ontology (GO) enrichment analysis for DEG datasets was performed by DAVID⁶⁰ with a cutoff of P<0.001. Interaction for genes related to cartilage and/or bone development, BMP signaling, TGFβ signaling, and Wnt signaling was searched using STRING database (https://string-db.org/) with high confidence score (≥0.7) and further analyzed using Cytoscape (www.cytoscape.org) on the basis of the degree of connectivity of the nodes. Gene Set Enrichment Analysis (GSEA) (www.gsea-msigdb.org/gsea/index.jsp)⁶¹ was applied with a background dataset consisting of all DEGs analyzed from cartilage and bone of the tibias of Mast4^{+/+} and Mast4^{-/-} mice at PN 1 day that were expressed >0.3 FPKM, which balances the numbers of false positives and false negatives⁶², in either Mast4^{+/+} or Mast4^{-/-} mice.

### Example 8—Reverse Transcription PCR (RT-PCR) and Real-Time RT-PCR

Total RNA was prepared using EasyBlue (Boca Scientific). 2 μg of RNA was reverse-transcribed using M-MLV Reverse Transcriptase (Promega) according to the manufacturer's instructions. RT-PCR was conducted using AccuPower™ PCR premix (Bioneer) with specific primer pairs. Quantitative real-time PCR was performed using Power SYBR Green PCR Master Mix (Applied Biosystems) on the QuantStudio 5 Real-Time PCR Instrument (Applied Biosystems). The mRNA levels of various genes were measured in triplicate and normalized with Gapdh. Information on the oligonucleotides used in this study is provided as

### Example 9—Histological Analysis

For cartilage immunofluorescence staining, the tissues were fixed with 4% paraformaldehyde (Wako) in 0.01M PBS (pH 7.4) overnight at 4° C., followed by decalcification using 10% EDTA solution. After being embedded in paraffin (Leica Biosystems), the samples were sectioned at a thickness of 6 μm. The tissue sections were incubated with the primary antibodies against Mast4 (Bioworld Technology), Col2a1 (Abcam), and Sox9 (Cell Signaling Technology) at 4° C. overnight. After washing in PBS, the tissue sections were consecutively incubated in AlexaFluor488 (Invitrogen) for 2 h at room temperature. Then the tissue sections were counter-stained with TO-PRO™-3 (Invitrogen) for 15 minutes. The images were taken using a confocal microscope DMi8 (Leica). To detect collagen tissue, sections were stained with freshly prepared Russell-Movat modified pentachrome (American MasterTech) according to the manufacturer's protocols. The images were made binary at a standard threshold, and the positive pixels were counted by using the Leica Microsystem CTR 6000 (Leica). For bone immunofluorescence staining, the mice were anesthetized and perfusion-fixed with 4% PFA to collect femurs and tibiae. The samples were fixed with 2% PFA at 4° C. overnight. The samples were decalcified in 0.5M EDTA solution for 6 days. Then, the samples were embedded into 5% low melting agarose (Invitrogen) and cut into 150 μm sections by vibratome (Leica, CT1200S). After removal of agarose from the sections, the sections were permeabilized with PBST (0.3% Triton X-100 in phosphate-buffered saline) for 20 minutes and blocked with 5% goat serum in PBST for 30 minutes. The sections were incubated with primary antibodies diluted in blocking solution at RT for 2 h, washed for 3 times with PBS and treated with secondary antibodies in blocking solution at RT for 75 minutes. After the sections were washed in PBST for 3 times and PBS for 3 times, the sections were mounted on microscope glass slides with fluorescence mounting medium (DAKO). Primary antibodies and reagents used for immunofluorescence were as follows: CD31 (Millipore, MAB1398Zm, 1:150), MMP13 (Abcam, 1:150), Osterix (Abcam, 1:300), Runx2 monoclonal (Cell Signaling Technology, 1:150). Secondary antibodies and reagents used for IF were as follows: FITC-conjugated anti-hamster IgG (Jackson ImmunoResearch, 1:300), Cy3-conjugated anti-rabbit IgG (Jackson ImmunoResearch, 1:300). Stained bone sections were analyzed at high resolution with a Zeiss LSM 880 confocal microscope (Carl Zeiss). Z-stacks of images were processed with Zen software.

### Example 10—Elemental Mapping by EPMA

An electron probe microanalyzer (EPMA-1610; Shimadzu, Kyoto, Japan) was used for the elemental mapping of Ca, P, and Mg. Undecalcified 6-week-old mouse tibias were embedded in epoxy resin and trimmed with diamond disks until exposure to a sagittal plane. After polishing, the specimens were sputter-coated with carbon before elemental analysis. For each experiment, 256×256 pixels mapping were performed. The accelerating voltage and beam current were set to 15 kV and 0.03 μA, respectively, and integrating time was 0.05 seconds at each pixel.

### Example 11—Micro CT

Three-dimensional reconstructed computed tomography images were obtained by scanning calcified SPC-generated bone regions with a MicroCT, Skyscan 1076 (Antwerp). The data were then digitalized using a frame grabber, and the resulting images were transmitted to a computer for analysis using Comprehensive TeX Archive Network (CTAN) topographic reconstruction software.

### Example 12—In Vivo Calcein labeling

Three-week-old mice were intraperitoneally injected with 50 mg/kg of calcein (Sigma-Aldrich, St. Louis, MO) in a 5% sodium bicarbonate solution. Mice were labeled 7 days and 2 days prior to sacrifice. Tibias were fixed in 4% paraformaldehyde for 1 day at RT. Samples were incubated in 10% (v/v) KOH for 96 h and embedded in paraffin, as previously described 63. Embedded samples were sectioned in 5 um thickness and visualized with confocal microscope (DMi8, Leica, Germany). Distance between the labels on cortical bone was measured at 3 points per sample.

### Example 13—Growth Plate Morphometry

For morphometric analysis, the total thickness of the growth plate cartilage at the proximal end of each tibia was measured at the H&E- or pentachrome-stained section images, equally spaced intervals along an axis oriented 90° to the transverse plane of the growth plate and parallel to the longitudinal axis of the bone. Three measurements were obtained from each epiphyseal growth plate, and final thickness determinations in individual animal indicated the average of these values using image-analysis software (ImageJ, ver. 1.38e, NIH, USA). The widths of the layers occupied by hypertrophic chondrocytes were measured by the same method. In addition, the percentage of the hypertrophic layer to the total thickness of the growth plate was calculated. Three left and right tibias were used for each group.

### Example 14—Isolation of Mouse Skeletal Stem Cells Using Flow Cytometry

FACS separation was performed, referring to the protocol²⁷. Briefly, male Mast4^{+/+} and Mast4^{-/-} mice (n=5) at 5 weeks of age were sacrificed, followed by dissection of humerus, femur and tibia. Cells were isolated with a combination of mechanical and chemical digestion, and red blood cells were removed by ammonium-chloride-potassium (ACK) lysis buffer. The TER119⁺CD45⁺ hematopoietic cells were filtered by magnetic-activated cell sorting (MACS). The remaining cells were then stained for the following antibodies: CD45, TER119, TIE2, ITGAV, CD202B, THY1.1, THY1.2, CD105, and 6C3. 7-AAD was used for live/dead cell discrimination. FACS analysis was performed on an FACS Aria ll Instrument (BD Biosciences) and analyzed by FlowJo v10.7.1 and BD FACSDiva v9.0.1 software.

### Example 15—In Vivo Cartilage Formation Assay

The control and Mast4-depleted C3H10T1/2 cells were cultured in the chondrogenic differentiation medium, including BMP-2 (150 ng/ml), for 4 days in a micromass culture. The cells were resuspended in PBS (100 micromass cultures in 100 μl per injection) and subcutaneously injected into the flanks of athymic nude mice (6-week old females; n=4). After 2 weeks, the mice were euthanized, and the grafts were collected for IHC evaluation. The volume of cartilage-containing grafts was measured and calculated using the formula V=(A*B²)/2, where V is volume (mm³), A is long diameter (mm), and B is short diameter (mm). All experiments were conducted in accordance with guidelines provided by the Institutional Animal Care and Use Committee of Center for Phenogenomics Animal Research Facility, Woojung BSC (Suwon, Korea, Association for Assessment and Accreditation of Laboratory Animal Care-accredited facility).

### Example 16—In Vivo Transplantation in Full-Thickness Cartilage Defect Rabbit Model

A full-thickness cartilage defect model was prepared as previously reported⁶⁴. Briefly, thirteen healthy New Zealand white male rabbits (3.0-3.5 kg in weight) were obtained 4 weeks before the experiment. The rabbits were anesthetized with Zoletil and xylazine. In sterile conditions, a parapatellar skin incision skin incision was made on the right knees, and the patella was dislocated laterally. Full-thickness osteochondral defects (3 mm in diameter and 3 mm in depth) were created at the center of the trochlear groove of the femur by drilling. Cartilage and bone debris were removed, and the defect sites were carefully washed with normal saline. Vehicle (PBS 50 μl), naïve or MAST4-depleted hBMSCs (2×10⁶ cells in 50 μl; passage 6-7) were transplanted into the defect sites (n=3 for vehicle, n=5 for naïve and MAST4-depleted hBMSCs), followed by relocation of the patella. The wound was closed with 4-0 nylon sutures. All procedures were conducted in accordance with guidelines provided by the Institutional Animal Care and Use Committee of KNOTUS Co., Ltd (Incheon, Korea).

### Example 17—Chromatin Immunoprecipitation

Cells were cross-linked with 1% formaldehyde for 10 minutes at room temperature. Glycine was added to a final concentration of 125 mM for 5 minutes to quench the formaldehyde crosslinks. Cells were washed with ice-cold phosphate buffered saline, harvested by scraping, pelleted, and resuspended in SDS lysis buffer (50 mM Tris-HCl [pH 8.1], 1% SDS, 10 mM EDTA) with complete protease inhibitor cocktail (Roche). Cell extracts were sonicated with a Bioruptor TOS-UCW-310-EX (output, 250 W; 23 cycles of sonication with 30-second intervals; Cosmo Bio). Samples were centrifuged at 18,472× g at 4° C. for 10 minutes, and the supernatants were diluted 10-fold in dilution buffer (20 mM Tris-HCl [pH 8.0], 2 mM EDTA, 1% Triton X-100, 150 mM NaCl, and complete protease inhibitor cocktail). Chromatin samples were precleared with protein A-agarose beads (Santa Cruz) for 2 h before immunoprecipitation against Sox9 and Smad3 (Abcam) antibodies overnight at 4° C. Immune complexes were collected with protein A-agarose beads. Samples were washed five times (first wash with low salt immune complex wash buffer [20 mM Tris-HCl, pH.8.0, 2 mM EDTA, 1% Triton X-100, 0.1% SDS, and 150 mM NaCl], second wash with high salt immune complex wash buffer [20 mM Tris-HCl, pH.8.0, 2 mM EDTA, 1% Triton X-100, 0.1% SDS, and 500 mM NaCl], third wash with LiCl immune complex wash buffer [10 mM Tris-HCl, pH.8.0, 1 mM EDTA, 250 mM LiCl, 1% NP-40, and 1% Na-deoxycholate], and the last two washes with TE buffer). Immunoprecipitated samples were eluted with buffer containing 1% SDS and 100 mM NaHCO₃ at room temperature. Eluates were heated overnight at 65° C. to reverse crosslinks after adding NaCl to a final concentration of 100 mM. Genomic DNA was extracted with a PCR purification kit (GeneAll). Precipitated chromatin by real-time PCR and the readouts were normalized using 5% input chromatin for each sample. The experiments were repeated two or more times. A forward primer of 5′-AACCCTGCCCGTATTTATTT-3′ (SEQ ID NO:25) and a reverse primer of 5′-TGTGCATTGTGGGAGAGG-3′ (SEQ ID NO:26) were used to detect the binding of Sox 9 to the Col2a1 gene. A forward of 5′-TGCTGACACTTTATTTTGCTCT-3′ (SEQ ID NO:27) and a reverse primer of 5′-CATCTCCAAGCCTCTTTCTG-3′ (SEQ ID NO:28) were used to detect the binding of Smad3 to the Mast4 gene.

### Example 18—Ubiquitination Assay

Flag-MAST4-PDZ, GFP-Smruf1, GFP-GSK-3β, and HA-Ub plasmids were transfected into C3H10T1/2 cells, followed by MG-132 treatment (10 μM for 6 h). Cells were lysed in SDS lysis buffer [10 mM Tris-HCl (pH 8.0), 150 mM NaCl, 1% SDS, 5 mM NEM, protease inhibitor] by boiling for 10 min, followed by 10-fold dilution with dilution buffer [10 mM Tris-HCl (pH 8.0), 150 mM NaCl, 1% Triton X-100]. Lysed samples were immunoprecipitated with Flag antibody (Sigma-Aldrich) overnight, and antibody-bound proteins were precipitated with Dynabeads. Washing buffer A [10 mM Tris-HCl (pH 8.0), 150 mM NaCl, 1% Triton X-100, 0.1% SDS] and B [10 mM Tris-HCl (pH 8.0), 150 mM NaCl, 1% Triton X-100] were used to wash precipitated samples, followed by western blotting.

### Example 19—Luciferase Assay

C3H10T1/2 cells were transiently transfected with 4xCol2a1-luc, Smad3/4-responsive promoter (CAGA)₁₂-luc, SBE-luc, 6xOSE-luc, MAST4-promoter luciferase report plasmids, HA-MAST4 PDZ, Myc-Sox9 WT/S494A/S494D plasmids using polyethylenimine (Polysciences). Cells were treated with TGF-β1 (3 ng/ml for 24 h) (R&D Systems) and Vactosertib (500 nM for 26 h). The luciferase activities were analyzed using the Luciferase Assay System kit (Promega) according to the manufacturer's protocol. All assays were done in triplicate, and all values were normalized for transfection efficiency against β-galactosidase activities.

### Example 20—Immunoprecipitation Assay and Western Blot Analysis

For the immunoprecipitation assay, cell extracts were incubated with the indicated primary antibodies overnight at 4° C. Antibody-bound proteins were precipitated with Dynabeads Protein G (Invitrogen). Cells were lysed in a RIPA buffer containing protease inhibitor cocktail (Complete; Roche). Samples were separated by SDS-PAGE, followed by electrotransfer to polyvinylidene difluoride membranes (PVDF; Millipore). The membrane was blocked for 1 h at room temperature and incubated overnight at 4° C. with the primary antibodies. Horseradish peroxidase-conjugated antibodies (Millipore) were used as secondary antibodies. The peroxidase reaction products were visualized with WESTZOL (Intron). All signals were detected by Amersham Imager 600 (GE Healthcare Life Sciences).

### Example 21—In-Gel Digestion for Mass Spectrometry Analysis Sample Preparation

The gel band corresponding to Myc-Sox9 size was excised and destained for 15 min with 50% (v/v) acetonitrile (ACN) prepared in 25 mM ammonium bicarbonate, and 100 mM ammonium bicarbonate sequentially. Proteins were reduced with 20 mM DTT at 60° C. for 1 h and then alkylated with 55 mM iodoacetamide at room temperature for 45 min in the dark. After dehydration, the proteins were digested with Trypsin/Lys-C Mix, mass spec grade (Promega, Madison, WI, USA) prepared in 50 mM ammonium bicarbonate overnight at 37° C. The peptides were extracted from the gel pieces with 50% (v/v) ACN prepared in 5% formic acid, dried under a Centrivap concentrator (Labconco, Kansas City, MO, USA), and stored at −20° C. until use.

### Example 22—Mass Spectrometry for the Detection of Phosphorylation of Sox9

The peptide samples extracted by in-gel digestion were suspended in 20 μl of solvent A (0.1% formic acid prepared in water, Optima LC/MS grade, ThermoFisher Scientific). Thereafter, 4 μl of the sample was loaded onto a EASYSpray C18 column (75 μm×50 cm, 2 μm) and separated with a 2-35% gradient of solvent B (0.1% formic acid prepared in ACN) for 65 min at a flow rate of 300 nL/min. Mass spectra were recorded on a Q Exactive hybrid quadrupole-Orbitrap mass spectrometer (Thermo Fisher Scientific) interfaced with a nano-ultraHPLC system (Easy-nLC1000; Thermo Scientific). The spray voltage was set to 1.5 kV and the temperature of the heated capillary was set to 250° C. The Q-Exactive was operated in data-dependent mode and each cycle of survey consisted of full MS scan at the mass range 300-1400 m/z and MS/MS scan for ten most intense ions. Exclusion time of previously fragmented peptides was for 20 sec. Peptides were fragmented using Higher energy collision dissociation and the normalized collision energy value was set at 27%. The resolutions of full MS scans and MS/MS scans were 70,000 and 17,500. The advanced gain control target was 5×10⁴, maximum injection time was 120 ms, and the isolation window was set to 3 m/z.

The raw data were processed by using the Trans-Proteomic Pipeline (v4.8.0 PHILAE) for converting to mzXML file which is search-available format. Database search for sequenced peptides was the Sequest (version 27) algorithm in the SORCERER (Sage-N Research, Milpitas) platform with Uniprot human database. Parent and fragment ion tolerance were set to 10 ppm (monoisotopic) and 1 Da (monoisotopic), respectively. Fixed modification was set on cysteine of 57 Da (carbamidomethylation). Variable modifications were set on methionine of 16 Da (oxidation) and on serine, threonine, tyrosine of 80 Da (phosphorylation). Trypsin was chosen as an enzyme with a maximum allowance of up to two missed cleavages. The Scaffold software package (version 3.4.9, Proteome Software Inc., Portland, OR, USA) was used to validate MS/MS-based peptide and protein identifications. The thresholds for peptide and protein identification were 95% minimum and 95% minimum, 2 peptides minimum, respectively. Peptide and protein FDR were 0.2% (Decoy) and 0.6% (Decoy).

### Example 23—Statistical Analyses

All quantitative experiments were performed in triplicate and/or repeated at least three times. Data were expressed as mean±SD. Student t tests was conducted using GraphPad Prism version 5 (GraphPad Software Inc.). P<0.05 was considered statistically significant. Significance was achieved at P<0.05.

### Example 24—Results

### Example 24.1—Mast4 Regulates Chondro-Osteogenic Gene Expression of MSCs

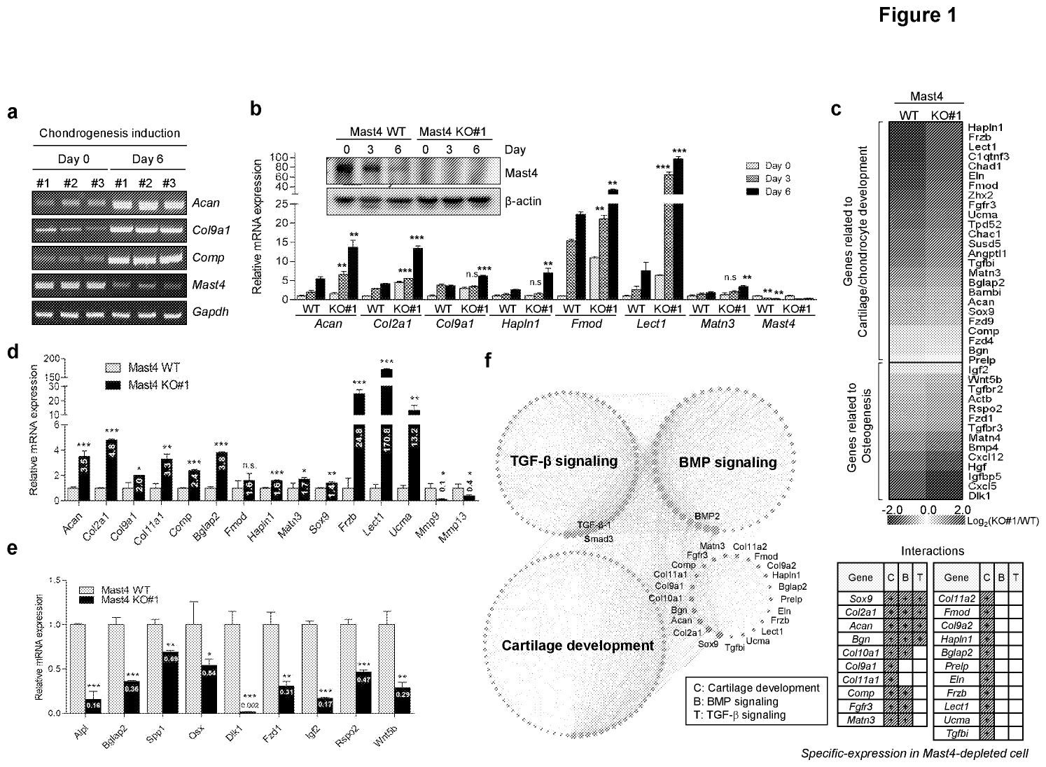

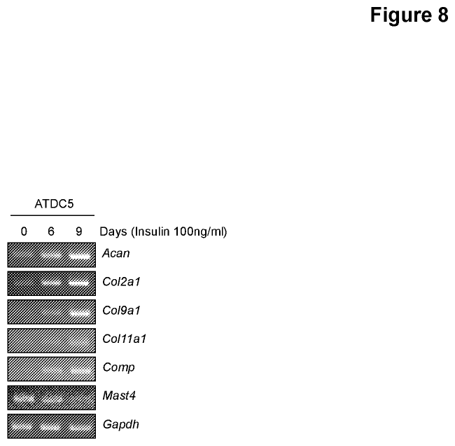

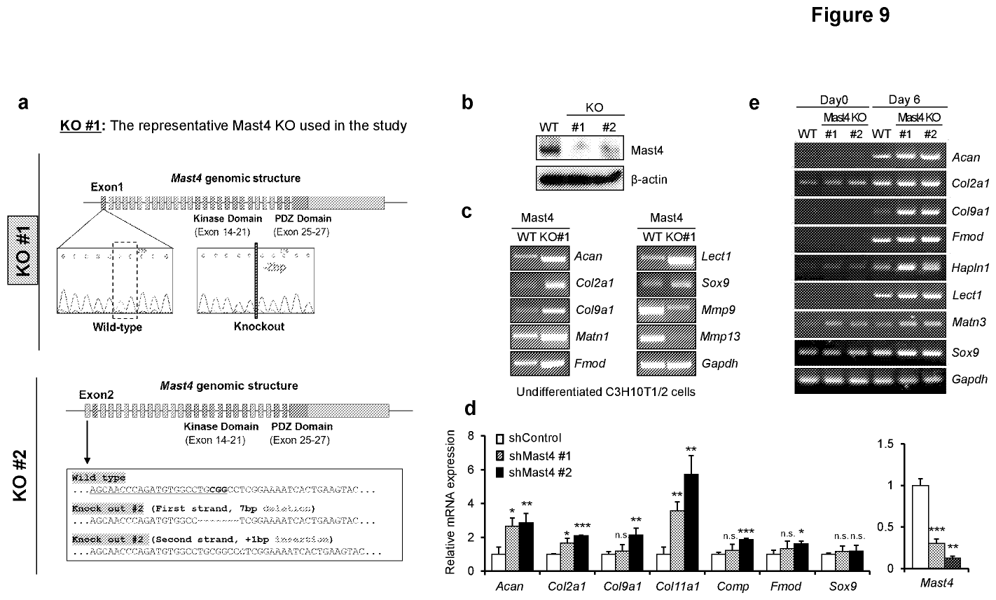

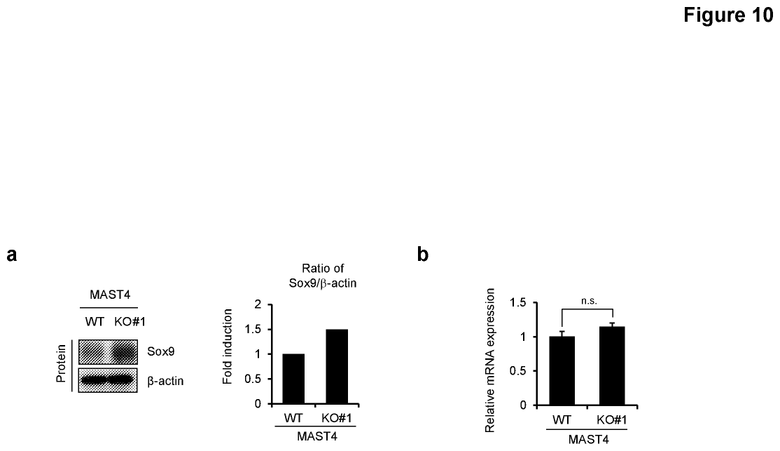

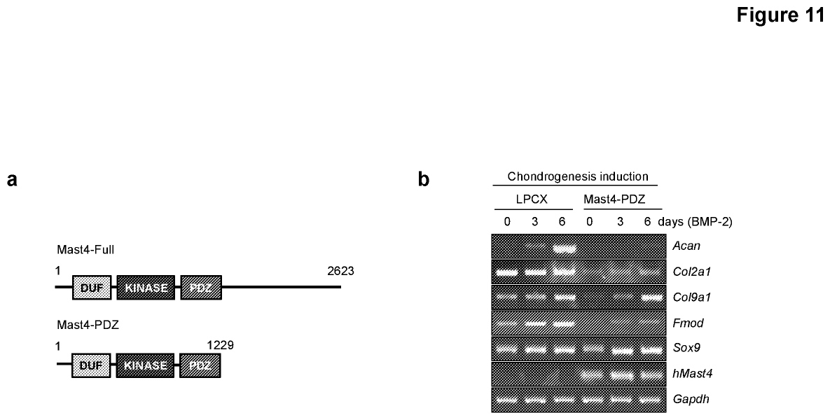

We identified microtubule-associated serine/threonine kinase 4 (Mast4) as one of the genes down-regulated during chondrogenic differentiation of C3H10T1/2 murine mesenchymal stromal cells and ATDC5 murine chondrogenic cells (FIG. 1a and FIG. 8). To investigate the role of Mast4 in MSC differentiation, we used the CRISPR/Cas9 system to disrupt Mast4 gene in C3H10T1/2 cells that underwent chondrogenic differentiation with the addition of BMP-2 or TGF-β1 (FIG. 9a,9b)²². We observed increased expression of cartilage-specific genes and reduced expression of matrix metallopeptidase (Mmp)-9/13 by CRISPR/Cas9-mediated Mast4 depletion and shRNA-mediated Mast4 knockdown in undifferentiated C3H10T1/2 cells (FIG. 9c,9d). Furthermore, during differentiation to chondrocytes under BMP-2 stimulation in high density micromass cultures, induction of cartilage matrix genes occurred earlier and was enhanced in Mast4-depleted C3H10T1/2 cells (FIG. 1b and FIG. 9d). Interestingly, we noticed that Mast4 depletion-mediated increase of Sox9 protein expression was more evident than that of Sox9 mRNA expression, implying post-translational regulation of Sox9 by Mast4 (FIG. 10). Since Sox9 is a key transcription factor for chondrocyte-specific genes, an increased amount of Sox9 protein induced by Mast4 depletion may help maintain the chondrocytic character of C3H10T1/2 cells. Next, we generated Mast4-overexpressing C3H10T1/2 cells to examine whether Mast4 inhibits chondrogenesis. Due to high molecular weight (>285 kDa) and relatively low expression of full-length Mast4, we alternatively used a truncated Mast4 construct (Mast4-PDZ) that contained a DUF, kinase, and PDZ domain (FIG. 11a). We found that forced expression of Mast4-PDZ protein efficiently suppressed the expression of chondrocyte marker genes (FIG. 11b).

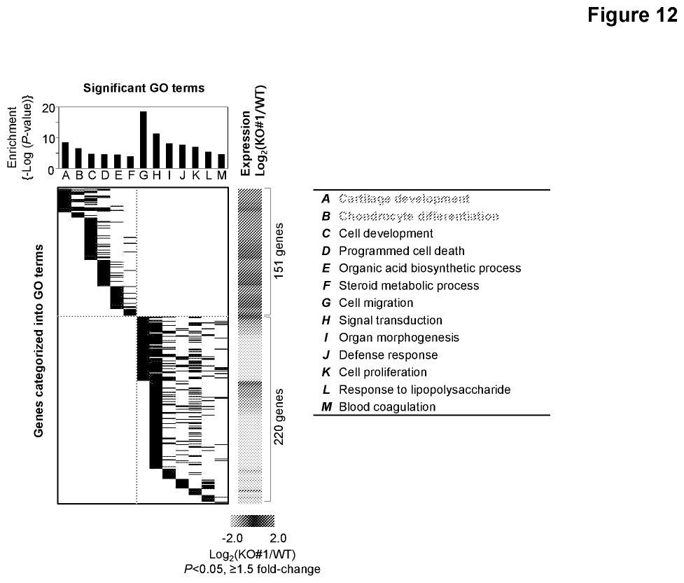

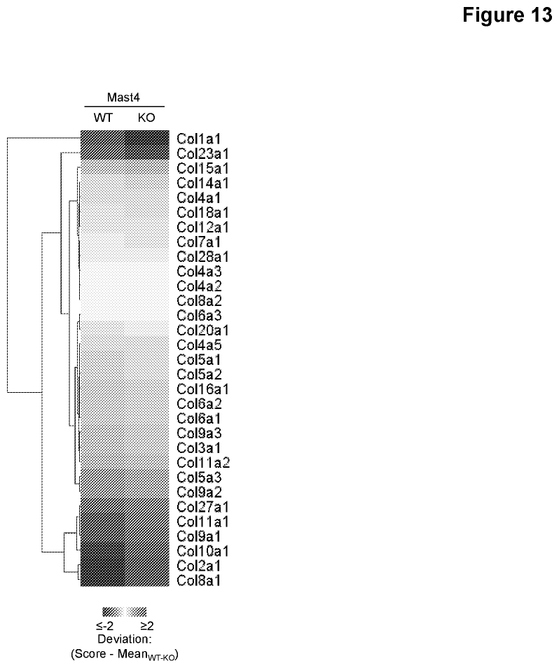

We further characterized the enhancement of chondrogenesis induced by Mast4 depletion by performing RNA sequencing using wild-type and Mast4-depleted C3H10T1/2 micromass cultures treated with BMP-2 for 6 days. Differentially expressed gene (DEG) analysis identified 151 up-regulated genes and 220 down-regulated genes in Mast4-depleted C3H10T1/2 cells (FIG. 12). Gene ontology enrichment analysis and the heatmap showed that genes related to chondrocyte differentiation (FDR=1.01×10⁻³) and cartilage development (FDR=5.05×10⁶were significantly enriched in Mast4-depleted cells (FIG. 12 and FIG. 1c). The expression of up-regulated genes involved in cartilage development (FIG. 1d) and down-regulated genes involved in bone formation (FIG. 1e) in the chondrogenic and osteogenic differentiated Mast4-depleted cells, respectively, were confirmed by qRT-PCR. Furthermore, among collagen genes, cartilage-specific collagen genes (Col2a1, Col9a1, Col9a2, Col9a3, Col11a1 and Col11a2) were specifically up-regulated by Mast4 depletion (FIG. 13). Particularly, 21 significantly up-regulated genes, related to cartilage and chondrocyte development, in Mast4-depleted cells highly interacted with the genes associated with BMP and TGF-β signaling pathways, which play important roles in chondrogenesis, besides cartilage development in a transcriptional network (FIG. 1f). Interestingly, Sox9 directly interacted with BMP2 and TGF-β1, being appeared to function as the hub of the network as larger nodes in the two signaling pathways. In summary, our data suggest that Mast4 may play a key role in regulating chondrogenic differentiation of MSCs.

### Example 24.2—Mast4 Modulates Chondrogenesis by Regulating Sox9 Stability

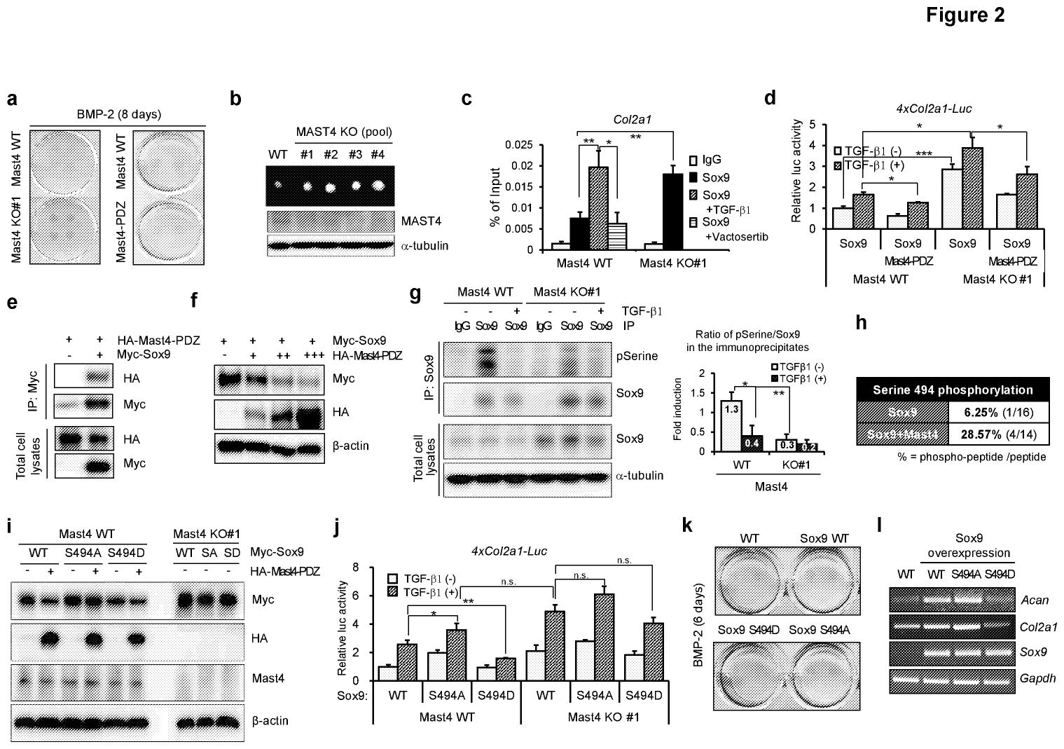

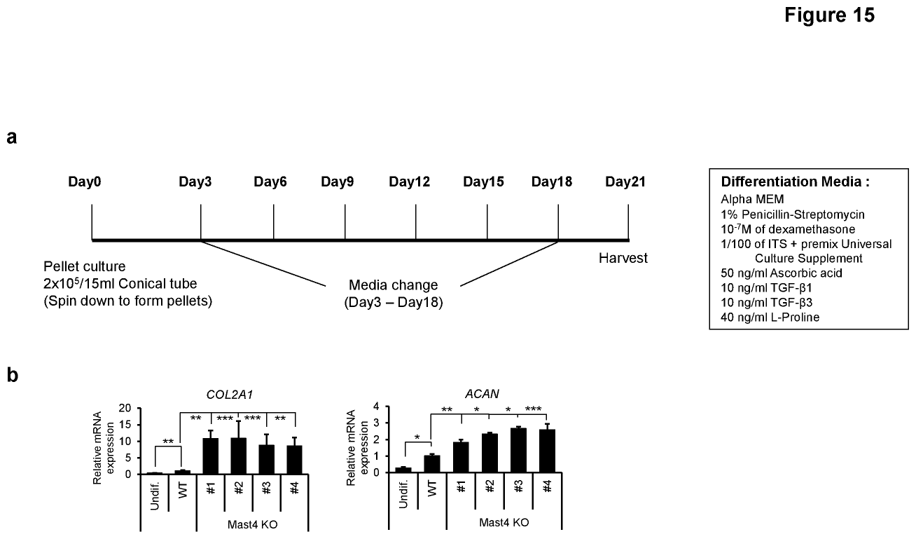

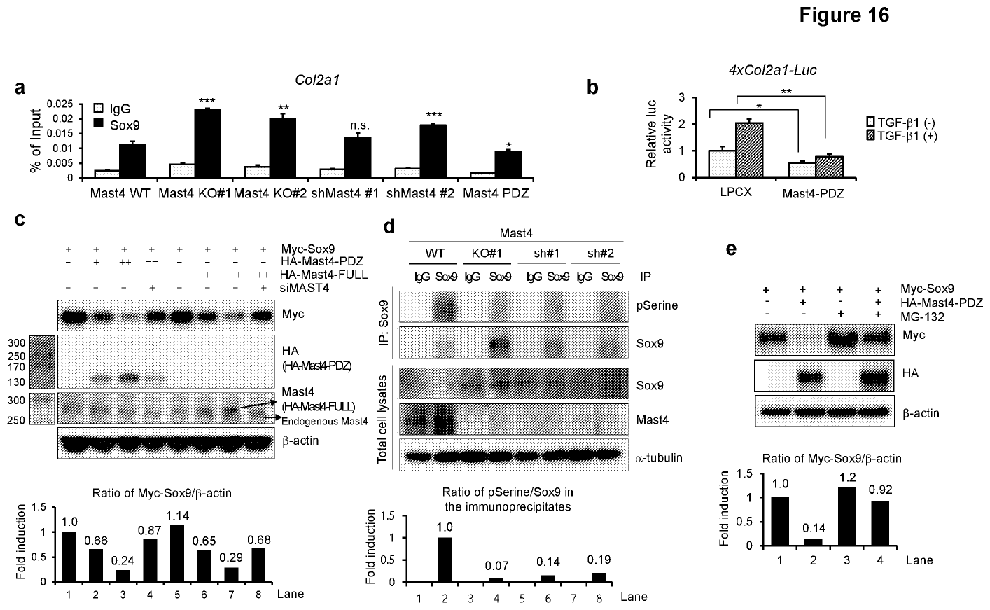

Next, we validated the effect of Mast4 on chondrogenesis of MSCs (FIG. 13a). The difference in cell growth was not significant between the wild-type and Mast4-depleted cells, whereas the cell growth was significantly increased in Mast4-PDZ-overexpressing C3H10T1/2 cells (FIG. 13b). However, alcian blue staining demonstrated that chondrogenic differentiation in Mast4-depleted cells was enhanced, whereas overexpression of Mast4-PDZ suppressed chondrogenic differentiation (FIG. 2a and FIG. 13c). The 3D spheroid formation assay using low-binding plates²³ also showed increased spheroid size and expression of cartilage-specific genes by Mast4 depletion (FIG. 13d,13e). In addition, chondrogenic differentiation of MAST4-depleted human bone marrow-derived stem cells (hBMSC) produced significantly increased size of cartilaginous aggregates and increased mRNA expression of COL2A1 and ACAN (FIG. 2b and FIG. 15). Chromatin immunoprecipitation (ChIP) assays further revealed that Mast4 deficiency significantly increased the binding of Sox9 protein to the promoter region of Col2a1 gene, which was reduced by Mast4 overexpression, during chondrogenic differentiation (FIG. 16a). Notably, Mast4 depletion was sufficient to considerably increase the binding of Sox9 protein to the promoter region of Col2a1 gene even in the absence of TGF-β1 treatment, which activates Col2a1 gene transcription through Sox9¹⁸ (FIG. 2c). On the other hand, Mast4-PDZ overexpression reduced basal and TGF-β1-induced Col2a1 promoter activity as well as Sox9-induced increase of Col2a1 promoter activity, which was more noticeable in Mast4-deficient cells (FIG. 16b and FIG. 2d).

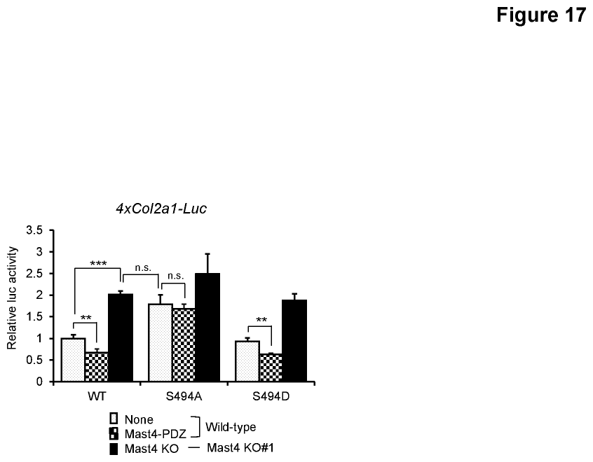

Our observation of the increase of Sox9 protein expression by Mast4 depletion (FIG. 10) prompted us to examine post-translational regulation of Sox9 protein stability by Mast4. Given that Mast4 functions as a serine/threonine kinase, we examined whether Mast4 induced Sox9 protein phosphorylation, leading to its protein degradation. We found that Mast4 bound to Sox9 and that overexpression of not only full-length but also truncated Mast4 decreased Sox9 protein expression, which was restored by blockade of Mast4 expression by siRNA, in a dose-dependent manner (FIG. 2e,2f and FIG. 16c). As expected, Sox9 serine phosphorylation was significantly reduced in Mast4-deficient cells, while basal Sox9 protein expression was increased, suggesting that Mast4 might promote Sox9 degradation by inducing Sox9 phosphorylation (FIG. 2g and FIG. 16d). Indeed, Mast4-induced Sox9 degradation was restored by MG-132 treatment (FIG. 16e). To identify the Mast4-mediated phosphorylation sites in Sox9 protein, we transiently overexpressed Sox9 and Mast4-PDZ proteins in C3H10T1/2 cells in the presence of MG-132 to prevent Mast4-mediated Sox9 degradation. And then Sox9 was immunoprecipitated, followed by MASS SPEC analysis. MASS SPEC analysis revealed serine 494 whose phosphorylation status was regulated by Mast4 (FIG. 2h). Particularly, we observed that Mast4-induced Sox9 degradation was more evident in C3H10T1/2 cells transfected with Sox9 WT and S494D substitution mutant of Sox9 protein which mimics phosphorylation, while S494A substitution mutant of Sox9 protein was barely degraded by Mast4 (FIG. 2i). We further found that luciferase activity of Sox9-induced Col2a1 promoter reporter by Sox9 S494A was increased in the wild-type cells, comparable to that by Sox9 WT in Mast4-depleted cells (FIG. 2j). Interestingly, the difference in Col2a1 promoter activities by Sox9 WT, S494A and S494D was not significant in the absence of Mast4. Besides, Mast4-PDZ-mediated reduction of Col2a1 activity was not significant in Sox9 S494A-transfected cells (FIG. 17). We further observed increased chondrogenesis of C3H10T1/2 cells stably overexpressing Sox9 S494A and decreased chondrogenesis of Sox9 S494D-overexpressing cells by alcian blue stain staining and RT-PCR for chondrocyte marker genes (FIG. 2k,2l). These results indicate that Mast4-mediated Sox9 protein phosphorylation at serine 494 may play an important role in chondrogenic differentiation of MSCs.

### Example 24.3—TGF-β-Induced Mast4 Suppression Enhances Sox9-Smad3 Association

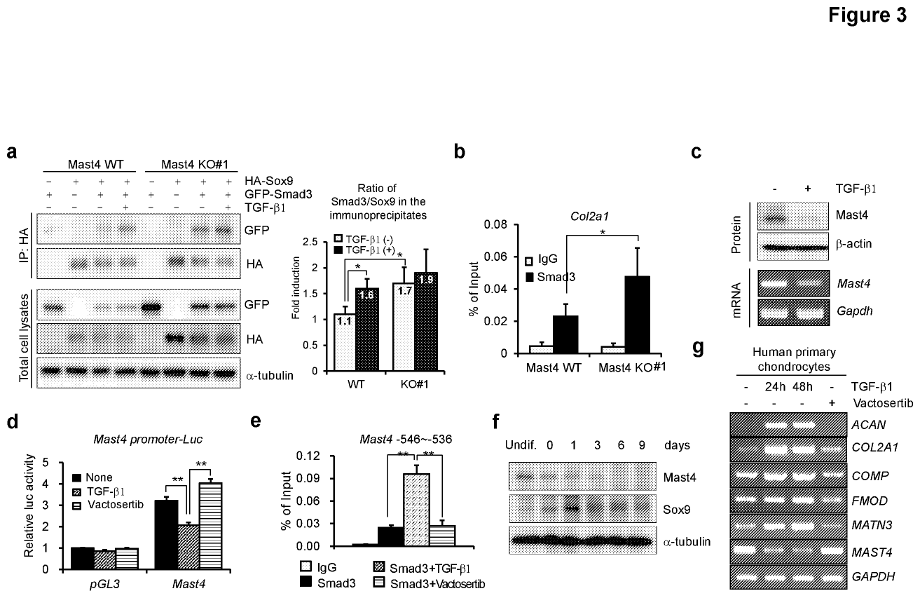

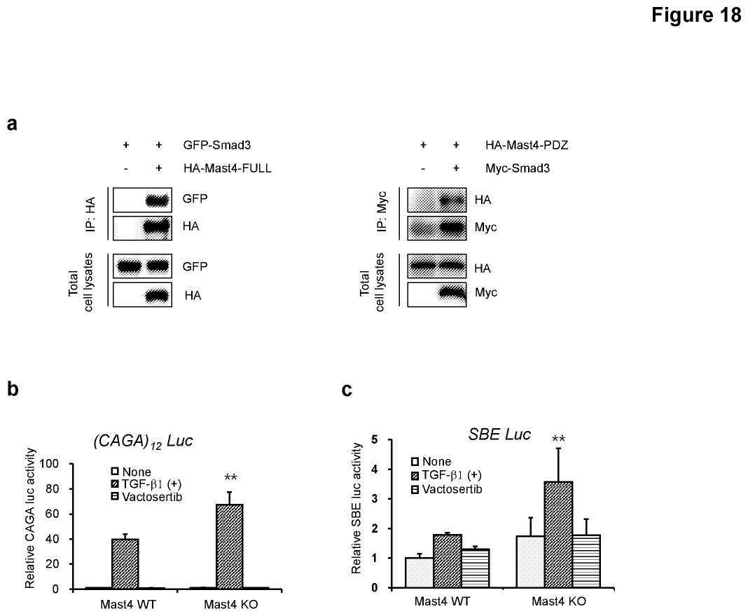

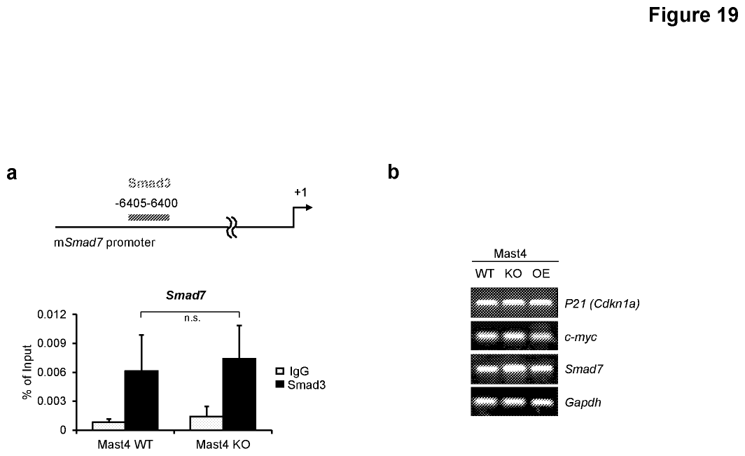

We have observed the interaction between Mast4 protein and Smad3 protein as well as increased TGF-β1/Smad3-induced transcriptional activity in Mast4-deficient cells (FIG. 18). In addition, a previous report demonstrated that Smad3 enhances Sox9-dependent transcriptional activation during chondrogenesis through interaction with Sox9¹⁸. Therefore, we hypothesized that Mast4 might suppress TGF-β1/Smad3-induced Sox9 transcriptional activity by facilitating Sox9 protein degradation, leading to decreased Smad3-Sox9 complex formation. Indeed, both Sox9 protein stability and Smad3-Sox9 association were increased by Mast4 depletion (FIG. 3a). Moreover, we found that Mast4 depletion elevated Smad3 occupancy at the Sox9-binding site of the Col2a1 gene (FIG. 3b). Importantly, the binding of Smad3 to the Smad7 promoter and expression levels of TGF-β1 cytostatic target genes, such as Cdkn1a, c-myc, and Smad7, were not affected by the status of Mast4 expression, indicating that TGF-β1-induced chondrogenic differentiation may rely upon the level of Mast4 which controls Sox9 protein stability (FIG. 19).

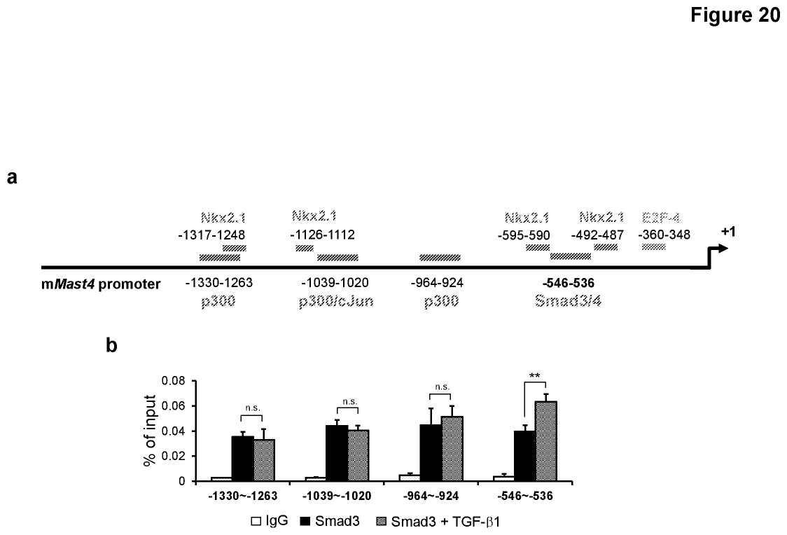

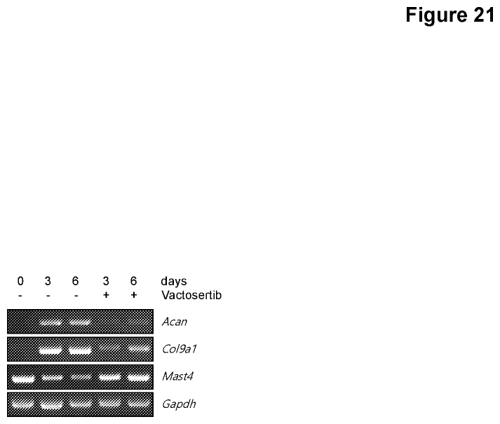

Considering the role of TGF-β1 signaling in chondrogenic differentiation¹², we investigated whether TGF-β1 induced chondrogenesis through regulation of Mast4 expression. Interestingly, TGF-β1 treatment markedly suppressed both mRNA and protein expression of Mast4 (FIG. 3c). Mast4 promoter activity was also suppressed by the TGF-β1 treatment, but Smad3 occupancy at the Mast4 promoter was significantly increased by TGF-β1 treatment, implying that TGF-β1/Smad3 signaling may negatively regulate Mast4 transcription (FIG. 3d,3e and FIG. 20). In particular, gradual reduction of Mast4 protein and mRNA expression level were observed during chondrogenic differentiation of C3H10T1/2 cells and in human primary chondrocytes treated with TGF-β1, respectively (FIG. 3f, 3g). These were well correlated with the increased Sox9 protein expression and mRNA expression of chondrocyte marker genes, reinforcing our findings of enhanced chondrogenesis of Mast4-deficient cells. Furthermore, inhibition of TGF-β signaling by the treatment of C3H10T1/2 cells with Vactosertib, a TGF-β receptor kinase inhibitor, prevented down-regulation of Mast4 gene and blocked induction of chondrocyte marker genes, indicating that suppression of Mast4 expression by TGF-β1 is essential for chondrogenic differentiation of MSCs (FIG. 21). Taken together, our observations suggest that TGF-β1/Smad3 signaling promotes chondrogenesis through suppression of Mast4 gene expression, resulting in subsequent accumulation of Sox9 protein.

### Example 24.4—Wnt-Induced Mast4 Stabilization Increases Osteogenesis of MSC

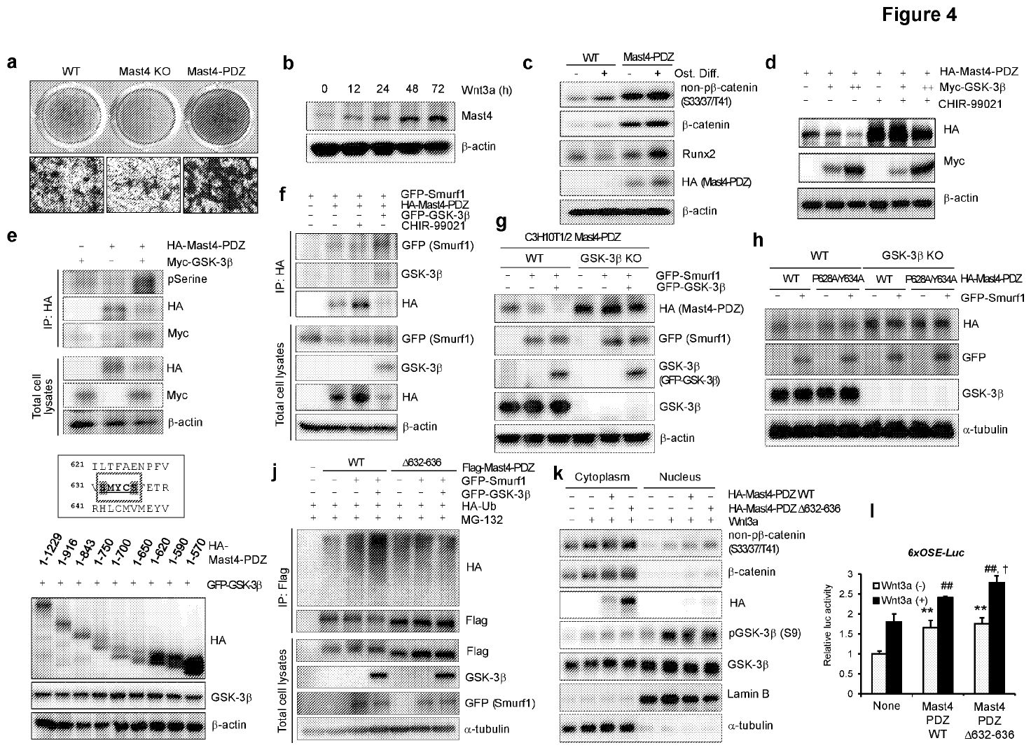

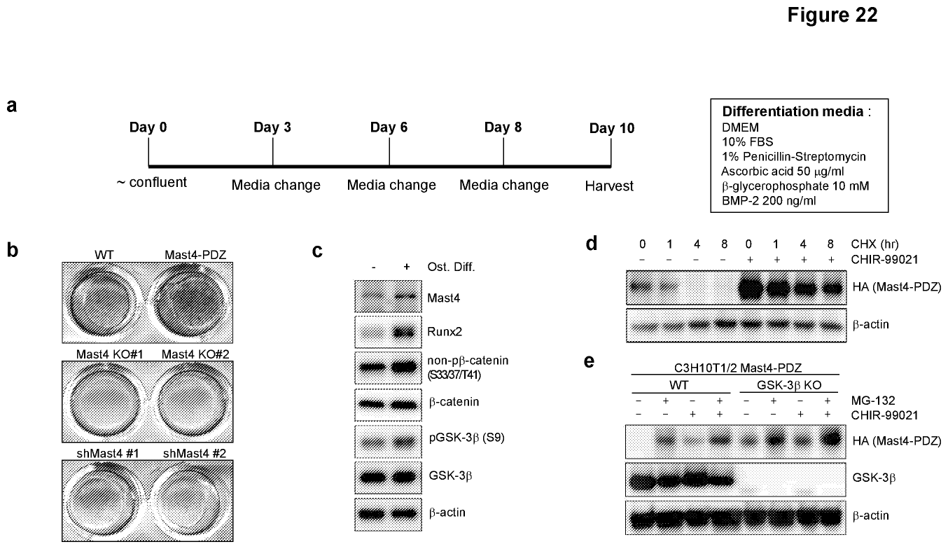

It is widely appreciated that Wnt/β-catenin signaling plays a critical role in skeletal development by governing the lineage commitment and differentiation of mesenchymal stromal cells into osteoblasts¹⁹. Our observation of down-regulation of the genes related to osteogenesis by Mast4 depletion in C3H10T1/2 cells led us to investigate whether Mast4 mediates Wnt/β-catenin-induced osteogenesis. Indeed, Alizarin Red S staining demonstrated enhanced osteogenic differentiation of C3H10T1/2 cells by stable overexpression of Mast4-PDZ (FIG. 4a and FIG. 22a,22b). Interestingly, Mast4 expression was elevated by Wnt3a stimulation in undifferentiated MC3T3-E1 pre-osteoblasts (FIG. 4b). Moreover, osteogenic differentiation of MC3T3-E1 cells led to increased Mast4 expression as well as increased Wnt signaling, shown by increased levels of active β-catenin and inactive GSK-3β (FIG. 22c). We further observed that stable overexpression of Mast4-PDZ significantly increased β-catenin expression and subsequent Runx2 expression during osteogenic differentiation of C3H10T1/2 cells (FIG. 4c). These observations were supported by RNA-seq analysis, demonstrating significant down-regulation of genes associated with osteogenesis in Mast4-depleted C3H10T1/2 cells (FIG. 1c).

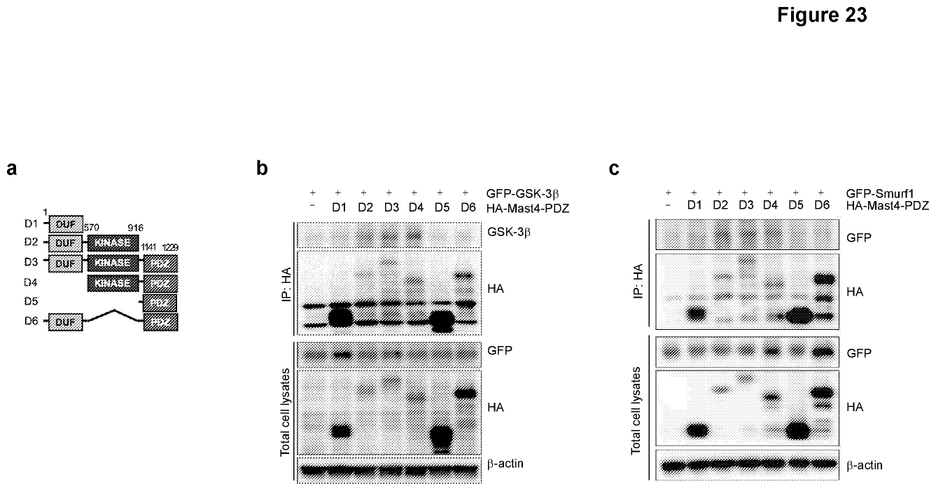

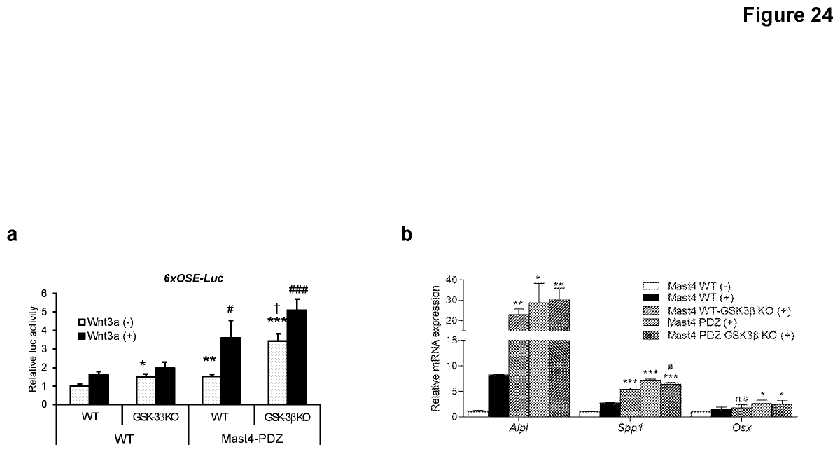

Having evidence for a role of Mast4 as a potent mediator of Wnt/β-catenin-induced osteogenic differentiation of progenitor cells, we found that Mast4 protein expression was decreased by GSK-3β in a dose-dependent manner and that GSK-3β inhibitor treatment dramatically increased Mast4 expression (FIG. 4d). Moreover, Mast4 degradation was significantly delayed by GSK-3β inhibition as well as by GSK-3β depletion (FIG. 22d,22e). Since GSK-3β- induced phosphorylation has been found to regulate the stability of numerous target proteins²⁴, we next addressed whether GSK-3β interacted with Mast4 to induce its degradation by phosphorylating Mast4. We observed that GSK-3β bound to the kinase domain of Mast4 and induced Mast4 serine phosphorylation (FIG. 23a,23b and FIG. 4e). Besides, among the reported E3 ubiquitin ligases that are recruited to target proteins in GSK3 phosphorylation-dependent manner²⁴, Smurf1 showed interaction with the kinase domain of Mast4 which intensity was regulated by GSK-3β status (FIG. 23c and FIG. 4f). We further observed that Smurf1-mediated Mast4 degradation was enhanced by GSK-3β overexpression, but blocked by GSK-3β depletion (FIG. 4g). Considering that WW domain-containing E3 ligases, including Smurf1, recognize the consensus PY motif (PPxY)²⁵, we found that mutation of PY-like motif in the kinase domain of Mast4 (P628A/Y634A) prevented Smurf1-mediated Mast4 degradation, which was no longer affected by GSK-3β depletion (FIG. 4h). To map the region responsible for the GSK-3β- mediated degradation of Mast4 in conjunction with Smurf1, we have generated serial deletion mutants of Mast4-PDZ (a.a.1-a.a.1229). As shown in FIG. 4i, expression levels of Mast4 deletion mutants containing a region between a.a.620 and a.a.1229 of Mast4-PDZ were significantly lower than those of Mast4 deletion mutants shorter than a.a.620 in the presence of GSK-3β, suggesting that a region between a.a.620 and a.a.1229 might contain the expected GSK3 consensus phosphorylation site (SxxxS^{P}). Interestingly, amino acid deletion at residues S632-S636 decreased Smurf1-mediated Mast4 polyubiquitination, which was enhanced by GSK-3β, while increasing Mast4 stability (FIG. 4j). Eventually, β-catenin nuclear localization and Runx2 transcriptional activity were increased by Mast4 overexpression and further enhanced by the blockade of GSK3β regulation of Mast4 through S632-S636 deletion (FIG. 4k,4l). Furthermore, either Mast4-PDZ overexpression or GSK-3β depletion resulted in significantly increased Runx2 transcriptional activity and subsequent osteogenic differentiation of C3H10T1/2 cells (FIG. 24). Taken together, our data suggest that Mast4, which is stabilized by Wnt-mediated GSK-3β inhibition, may function as an important mediator of Wnt/β-catenin-induced osteogenesis.

### Example 24.5—Skeletal Abnormalities in Mast4^{-/-} Mice

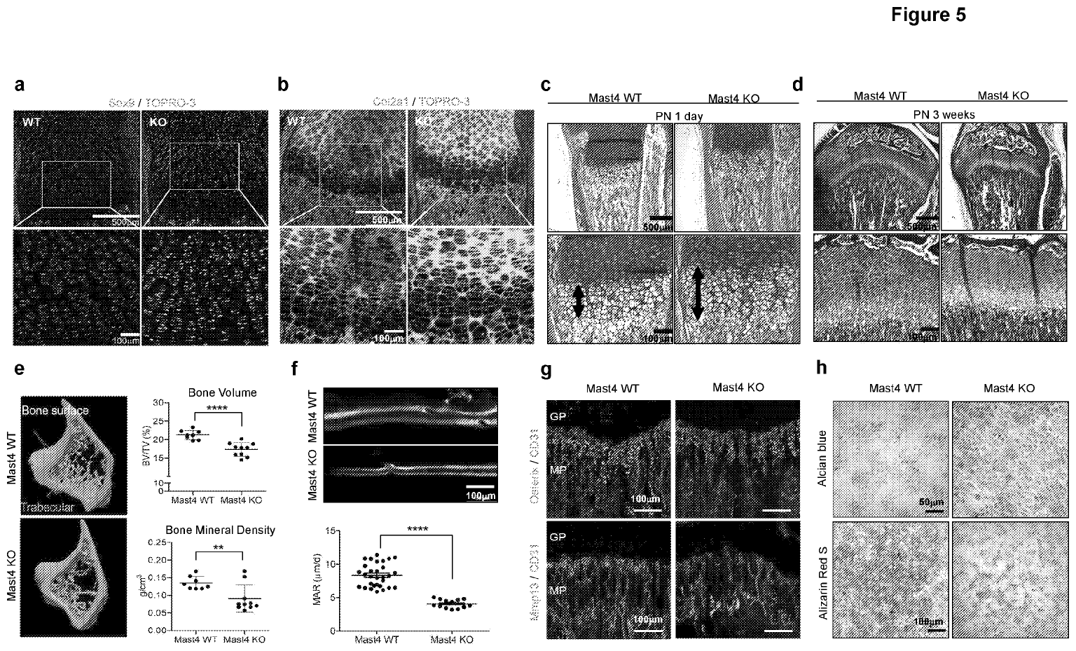

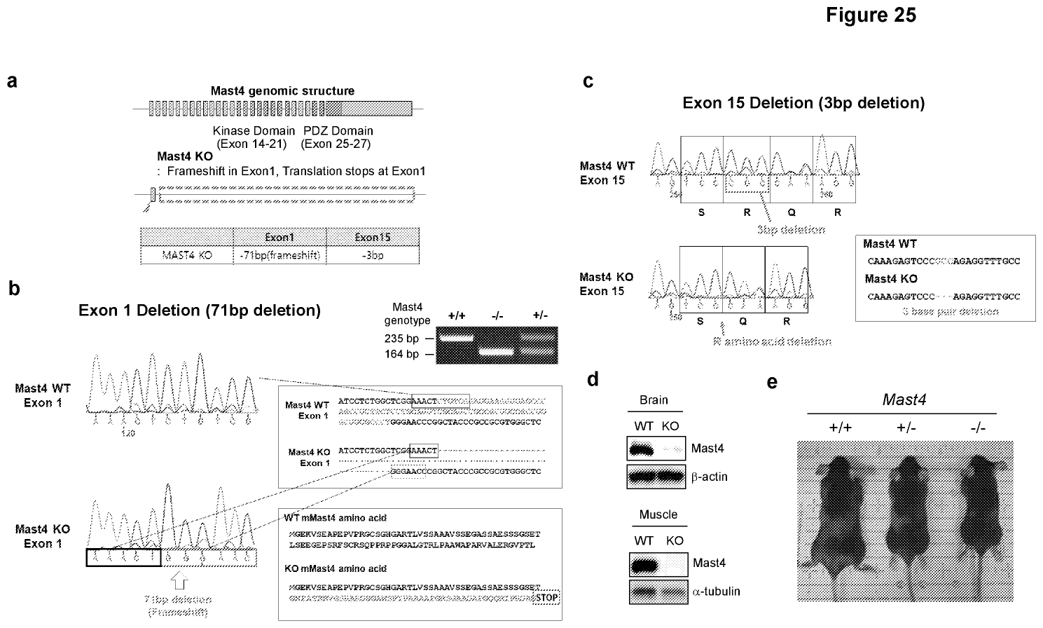

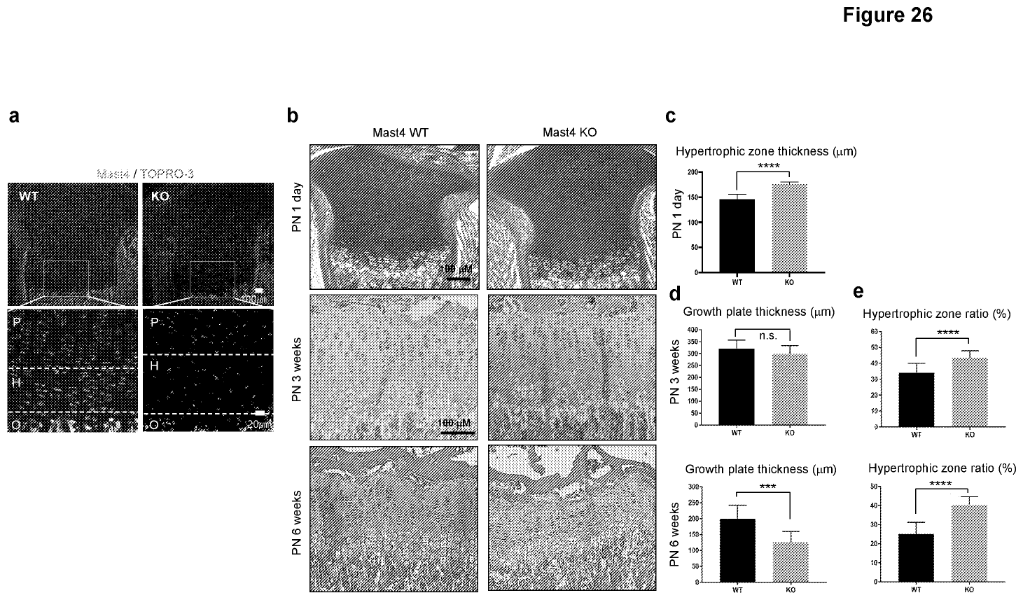

To examine the role of Mast4 in MSC differentiation, we generated Mast4^{-/-} mice using CRISPR/Cas9-mediated knockout system (FIG. 25a-25d). Even though Mast4^{-/-} mice were viable, they became smaller in size, compared to the wild-type littermates (FIG. 25e). Although we cannot exclude the possibility that Mast4 effects on other organs also contribute to the small size of the mice, we focused on investigating whether disruption of the Mast4 gene affects differentiation of MSCs during endochondral ossification in the long bones since MSCs are found in many internal organs of adult mice. The expression of Mast4 was observed throughout the tibial growth plate of the Mast4^{+/+} mice at postnatal (PN) 1 day (FIG. 26a). Interestingly, increased expression of Mast4 was observed in hypertrophic chondrocytes, while a smaller portion was localized in proliferating chondrocytes in the tibias. This observation supports our findings regarding enhanced chondrogenic differentiation of C3H10T1/2 cells by Mast4 depletion and suggests a role of Mast4 in mediating a switch to bone growth in the terminal stage of chondrocyte differentiation. Indeed, stronger expression of Sox9 and Col2a1 proteins, two key markers of chondrocytes, was observed in the growth plates of Mast4^{-/-} mice at PN 1 day (FIG. 5a,5b). Pentachrome staining also displayed an increased hypertrophic layer in the proximal epiphysis of Mast4^{-/-} mice at PN 1 day (FIG. 5c and FIG. 26b,26c). Furthermore, more yellowish pentachrome staining was detected in the growth plates of Mast4^{-/-} mice at PN 3 weeks, suggesting that Mast4 depletion may contribute to collagen synthesis in the growth plate (FIG. 5d). The tibial growth plate thickness of Mast4^{-/-} mice showed no significant difference between Mast4^{+/+} mice at PN 1 day and 3 weeks, while being significantly reduced at PN 6 weeks (FIG. 26b,26d). However, the ratio of hypertrophic layer to the total thickness of the growth plate was significantly increased in Mast4^{-/-} mice at PN 3 and 6 weeks (FIG. 26e). This observation suggests that excessive cartilage synthesis in hypertrophic zone of the growth plate of Mast4^{-/-} mice resulted in reduced proliferation of the growth plate, leading to abnormal ossification. Since the increased hypertrophic layer is the major phenotype observed in both Mmp9^{-/-} and Mmp13^{-/-} mice²⁶, increased hypertrophic layer in Mast4^{-/-} mice may be associated with down-regulation of Mmp9 and Mmp13 observed in Mast4-depleted C3H10T1/2 cells (FIG. 1d and FIG. 9c).

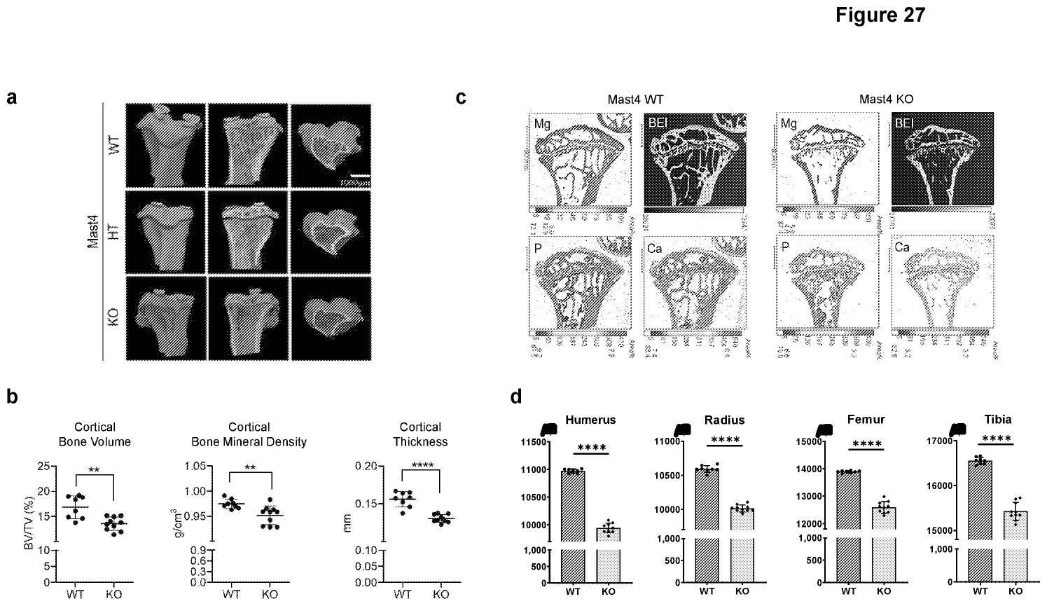

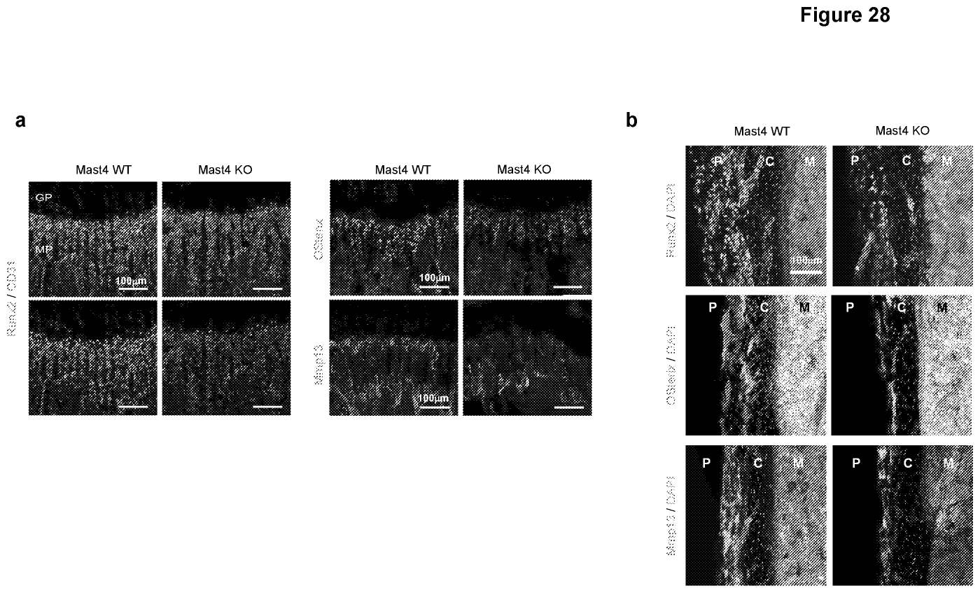

On the other hand, the μCT analyses of Mast4^{-/-} mice demonstrated an osteoporotic phenotype with significantly reduced metaphyseal trabecular bones, more porous and thinner cortical bones, and decreased bone volume and mineral density (FIG. 5e and FIG. 27a, 27b). Furthermore, the electron probe microanalyzer (EPMA) exhibited lower levels of critical mineral ions for bone development and bone health, such as magnesium (Mg), phosphate (P) and calcium (Ca), in the tibias of 6-week-old Mast4^{-/-} mice (FIG. 27c). In particular, we observed that Mast4^{-/-} mice showed significantly decreased bone formation, measured by double calcein labeling, and shorter limb length compared to Mast4^{+/+} mice (FIG. 5f and FIG. 27d). Consistently, reduced expression of Osterix, an osteoblast marker crucial for bone formation, and Mmp13, a marker for mature osteoblasts and a target of Osterix, were observed in the proximal tibias and distal femurs of Mast4^{-/-} mice at PN 1 day (FIG. 5g and FIG. 28).

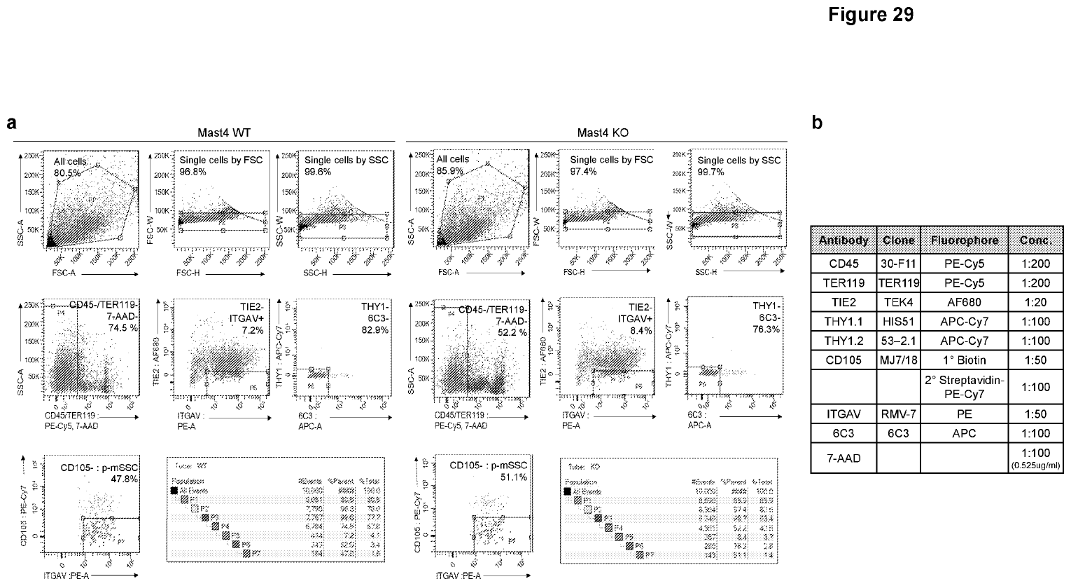

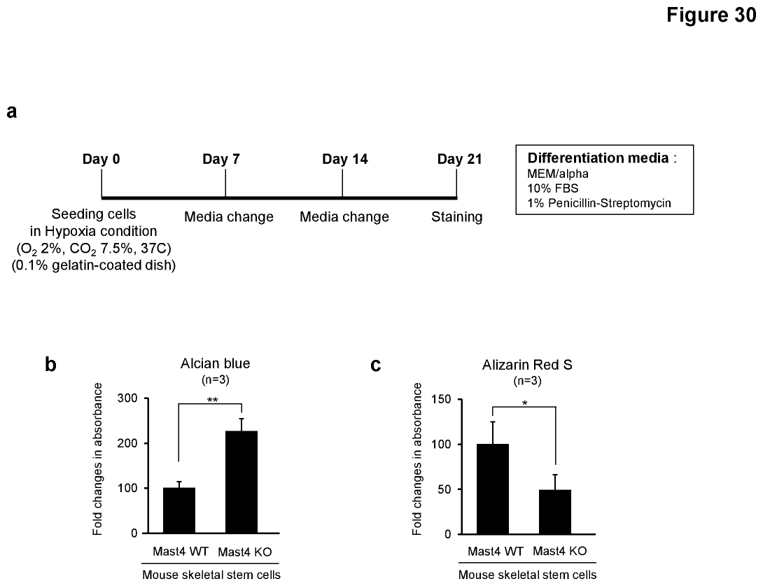

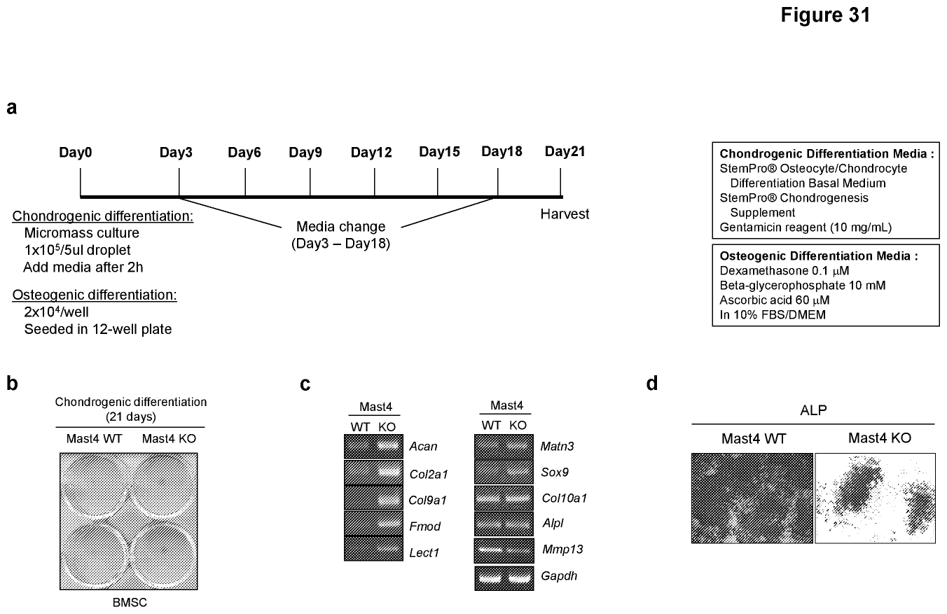

We further isolated skeletal stem cells, a purified population of CD45⁻TER119⁻TIE2⁻ITGAV⁺THY1⁻6C3⁻CD105⁻, from Mast4^{+/+} mice and Mast4^{-/-} mice using the expression of cell surface markers²⁷ (FIG. 29). Functional assessment of these stem cells by in vitro colony formation assay revealed enhanced chondrogenic differentiation, shown by stronger alcian blue staining intensity, but suppressed osteogenic differentiation, shown by weaker Alizarin Red S staining intensity, abilities of the skeletal stem cells of Mast4^{-/-} mice (FIG. 5h and FIG. 30). We also isolated bone marrow-derived stem cells (BMSC) and induced chondrogenic and osteogenic differentiation. Consistently, enhanced chondrogenic but reduced osteogenic differentiation was observed in BMSCs of Mast4^{-/-} mice (FIG. 31). Collectively, our data suggest that Mast4 regulates the fate of MSC differentiation into cartilage or bone in vivo.

### Example 24.6—Phenotype of Cartilage in the Tibias of Mast4^{-/-} Mice

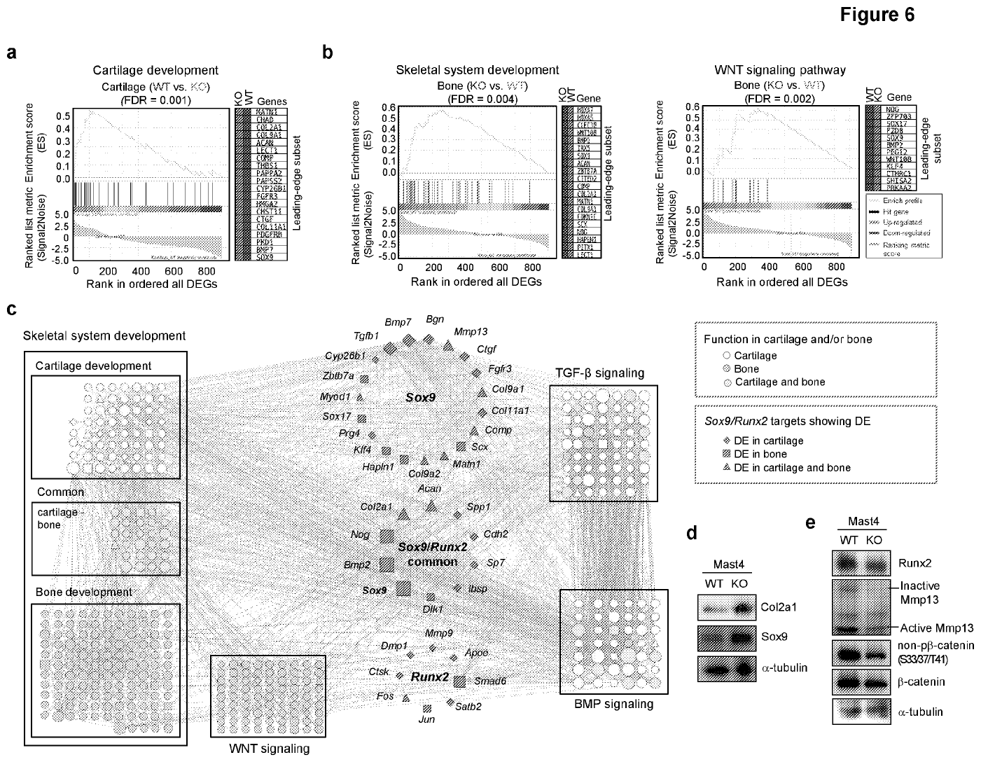

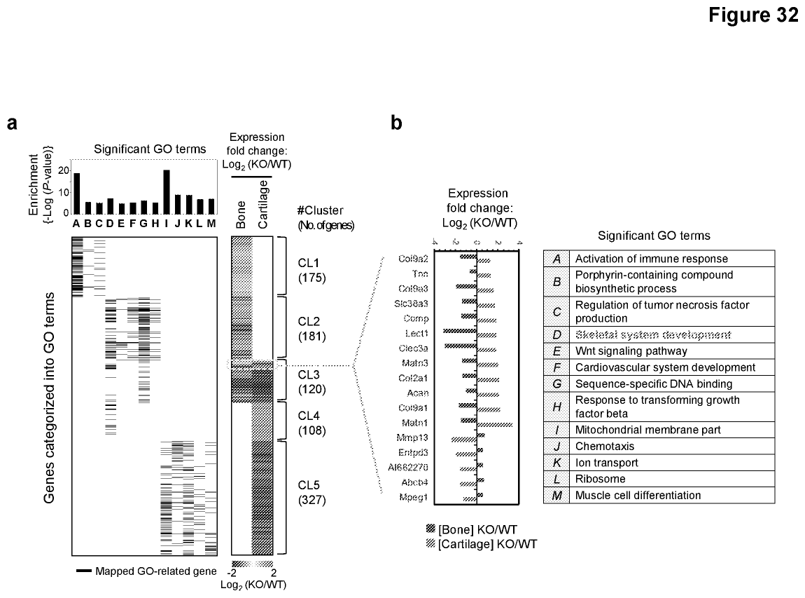

To gain a better understanding of the role of Mast4 in MSC differentiation, we conducted RNA sequencing by collecting and combining RNAs obtained from bone and cartilage of the tibias of Mast4^{-/-} mice at PN 1 day with those of wild-type mice (3 mice per each group). Differentially expression (DE) analysis exhibited tissue-specific expression with 175 up-regulated (CL1) and 181 down-regulated (CL2) genes in bone, and 108 up-regulated (CL4) and 327 down-regulated (CL5) genes in cartilage of Mast4^{-/-} mice (FIG. 32a). Gene ontology (GO) enrichment analysis revealed the high association with skeletal system development (D in FIG. 32b; represented by CL2, CL3, and CL4; P=4.5×10⁻⁸˜9.3×10⁻⁴). Remarkably, 17 of the 120 common differentially expressed genes (DEGs; CL3), mostly identified as Sox9 target genes that are cartilage-specific for skeletal system development, showed distinct switches in expression between bone and cartilage (FIG. 32b), suggesting the functional involvement in MSC differentiation. Gene set enrichment analysis for DEGs also revealed that significant numbers of DEGs in cartilage were assigned to cartilage development (FDR=0.001), while DEGs in bone were assigned to skeletal system development (FDR=0.004) and Wnt signaling pathway (FDR=0.002) (FIG. 6a,6b). Particularly, most of Sox9 target genes were up-regulated in cartilage of Mast4^{-/-} mice, but not in bone. A network of Sox9 and Runx2 target genes showing differential expression in cartilage and/or bone of Mast4^{-/-} mice were analyzed. These Sox9 and Runx2 targets were highly interacted with the genes related to skeletal system development, including cartilage and bone development, TGF-β signaling, BMP signaling, and Wnt signaling (FIG. 6c). Genes related to cartilage development were highly interacted with Sox9 target genes (i.e. Tgfb1, Bmp7 and Bgn; DE in cartilage). However, genes related to bone development and Wnt signaling were mostly interacted with the common target genes of Sox9 and Runx2 (i.e. Nog, Bmp2 and Sox9; DE in bone), or Runx2 targets (i.e. Smad6; DE in bone). Interestingly, the network of Sox9 target genes showed the regulation of collagen family and CL3-switch genes in bone and cartilage. Collectively, our results suggest that Mast4 is a key regulator of a transcriptional network associated with skeletal system development.

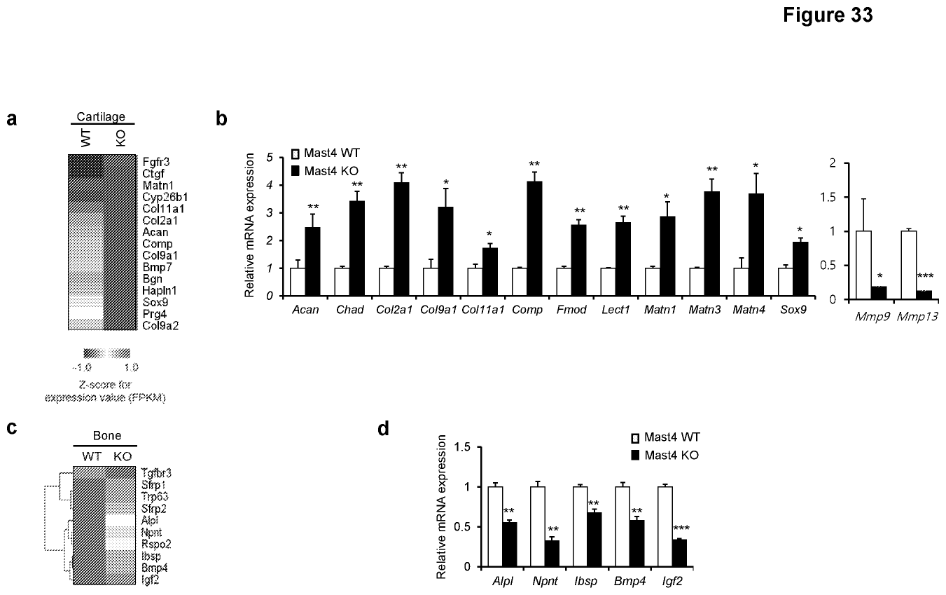

Next, expression of the selected cartilage matrix genes and Sox9 target genes in the transcriptional network was further examined in cartilage tissues isolated from the tibias of wild-type and Mast4^{-/-} mice at PN 1 day by qRT-PCR (FIG. 33a,33b). Particularly, collagenase genes (Mmp9 and Mmp13) were down-regulated, supporting our observation of increased hypertrophic layer in Mast4^{-/-} mice (FIG. 5c). We also found that mRNA expression of the genes associated with osteoblast differentiation was decreased in the bone tissues of Mast4^{-/-} mice (FIG. 33c,33d). Consistent with these observations, the increase of Sox9 and Col2a1 and the decrease of β-catenin, Runx2, and Mmp13 protein expressions were observed in the cartilage and bone tissues, respectively, of Mast4^{-/-} mice (FIG. 6d,6e). Taken together, our results of molecular phenotype of Mast4^{-/-} mice support that Mast4 regulates the expression of the aforementioned genes through modulation of Sox9, ultimately facilitating chondrogenesis in vivo.

### Example 24.7—Mast4 Depletion Facilitates Cartilage Formation and Repair

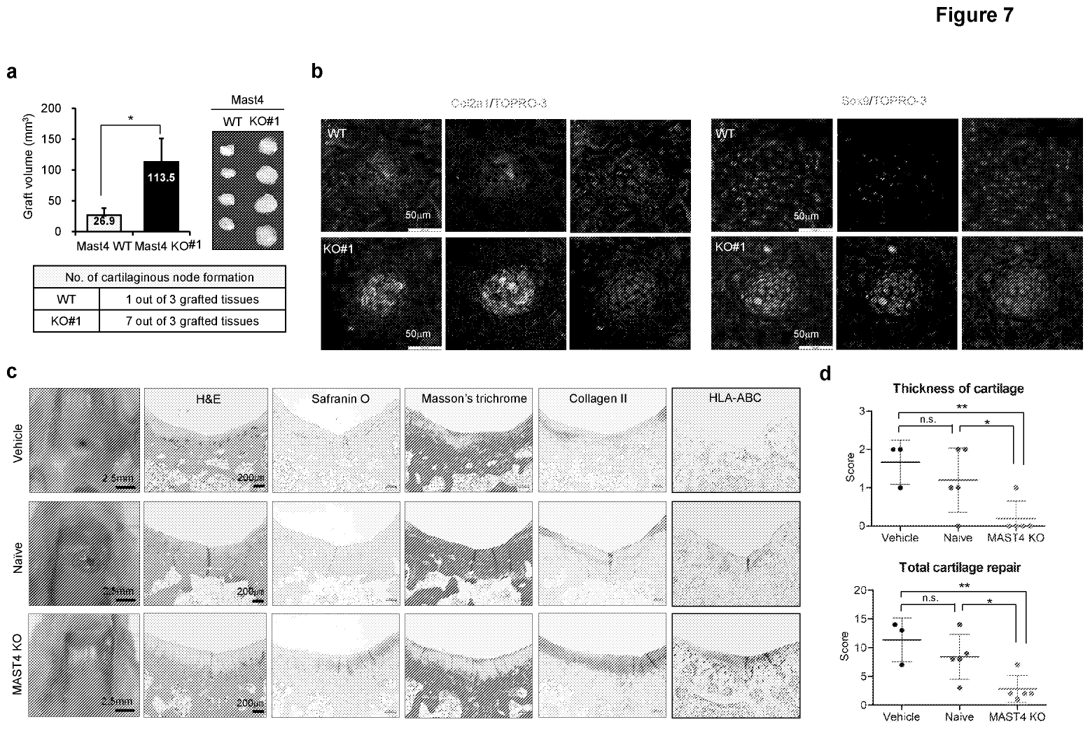

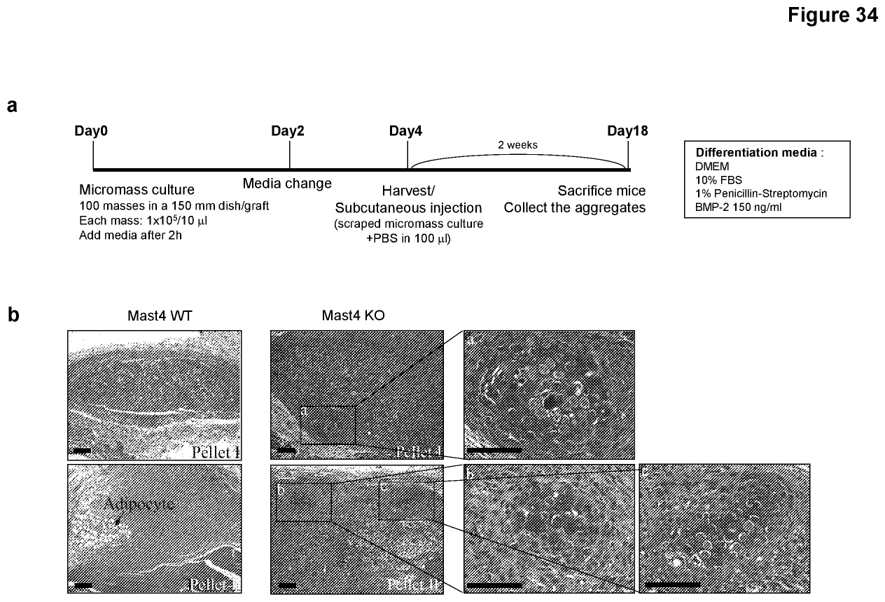

To further investigate the effect of Mast4 depletion on chondrogenesis in vivo, differentiated wild-type and Mast4-depleted C3H10T1/2 micromass cultures were subcutaneously implanted into nude mice for assessment of cartilage formation (FIG. 34a). After 2 weeks, the grafted tissues formed by Mast4-depleted cells showed a significant increase in volume (FIG. 7a). Previous reports showed that cartilage formation from implantation of MSCs was barely observed by subcutaneous implantation, while it was more commonly observed by using diffusion chamber²⁸ or implanting inside a cartilaginous defects²⁹. Similar to these reports, the grafts formed by both wild-type and Mast4-depleted C3H10T1/2 cells were not comprised of high portion of cartilaginous nodes. However, we found an increased incidence of cartilaginous node formation in the grafts formed by Mast4-depleted cells. To confirm whether the nodes were comprised of cartilage, tissue sections were stained with pentachrome (FIG. 34b). An increased collagen deposition around the nodes was shown in the grafts formed by Mast4-depleted cells. Moreover, Col2a1, a chondrogenic marker protein, and Sox9 proteins were highly expressed in the grafts formed by Mast4-depleted cells (FIG. 7b). Taken together, these results imply that depletion of Mast4 may enhance chondrogenic commitment of MSCs in vivo.

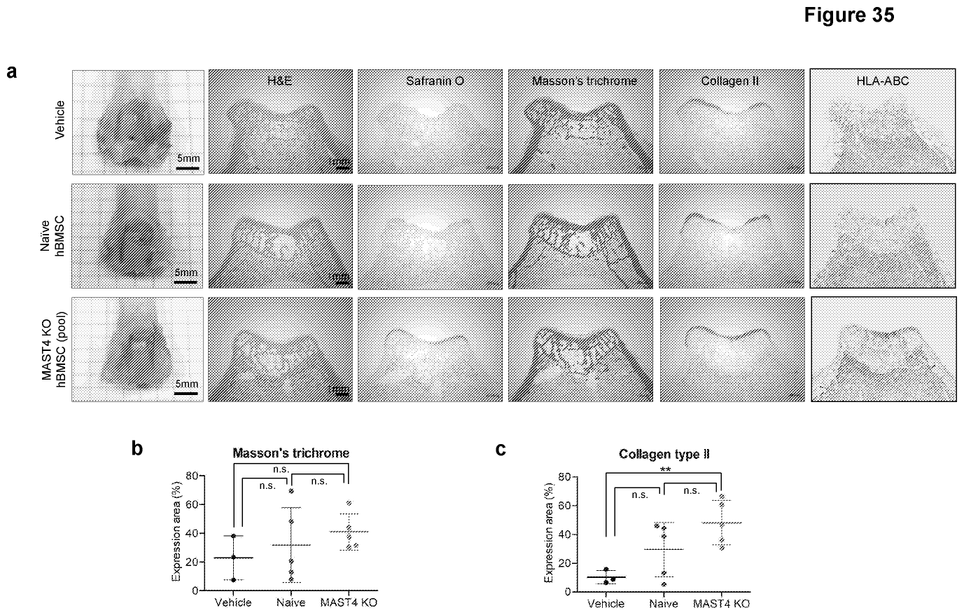

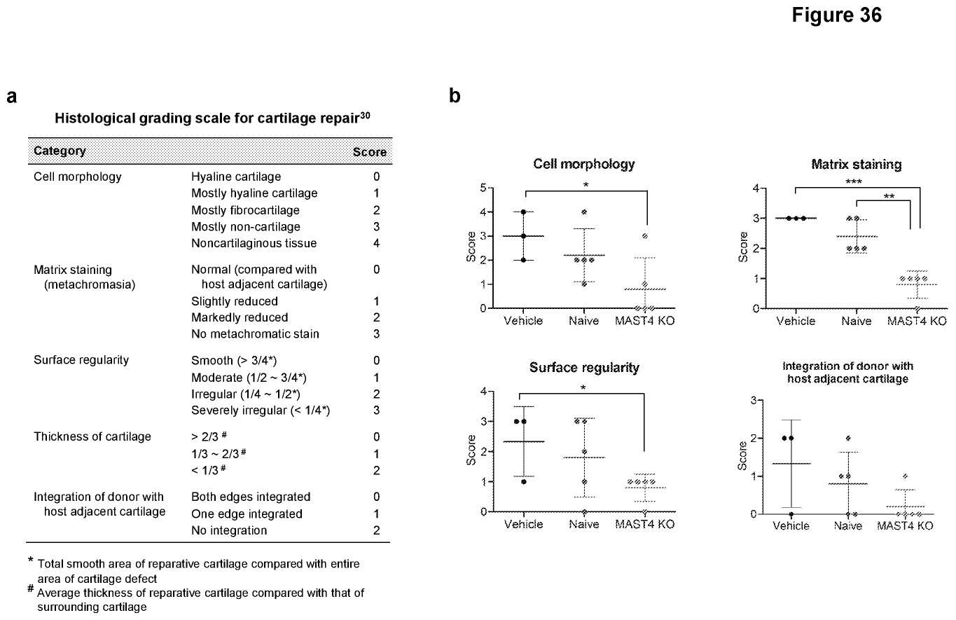

Next, we assessed the effect of MAST4 depletion in human bone marrow-derived stem cells (hBMSC) on cartilage repair in a rabbit full-thickness cartilage defect model. Since hBMSC were unable to form colonies from individual cells, the pools of CRISPR/Cas9-mediated MAST4-depleted cells showing at least 70% of indel, frameshift or 21+ bp indel were used. No abnormal findings or severe inflammatory reactions were observed. While the defects in the knees treated with vehicle (PBS) or transplanted with naïve hBMSCs were vacant and distinguishable from the surrounding tissues, the knees transplanted with MAST4-depleted hBMSCs exhibited smooth white repaired tissues which covered the defect without showing obvious margin with the normal surrounding cartilage (FIG. 7c and FIG. 35). The architecture of the repaired tissues in the MAST4-depleted hBMSCs-transplanted knees resembled those of normal surrounding cartilage, whereas those in the naïve hBMSCs-transplanted knees showed irregular surface and noticeable gaps in the border area. In addition, increased Safranin O and Massons' trichrome staining were noted in the repaired tissues of MAST4-depleted hBMSCs-transplanted knees without significant gaps, indicating enhanced production of cartilage tissues and collagen matrix, respectively. Immunohistochemical staining against type II collagen further revealed significantly strong expression and similar density with the surrounding normal tissues by transplantation of MAST4-depleted hBMSCs. Overall, the modified Wakitani score analysis³⁰ demonstrated that the transplantation of MAST4-depleted hBMSCs to the defect sites significantly improved cartilage repair and regeneration (FIG. 7d and FIG. 36). Collectively, these results suggest that Mast4 depletion may be a useful strategy for MSC-based therapy for cartilage regeneration.

### REFERENCES

1 Pittenger, M. F. et al. Multilineage potential of adult human mesenchymal stem cells. Science (New York, N.Y.) 284, 143-147 (1999).
2 Chen, Q. et al. Fate decision of mesenchymal stem cells: adipocytes or osteoblasts? Cell Death & Differentiation 23, 1128-1139, doi:10.1038/cdd.2015.168 (2016).
3 Bell, D. M. et al. SOX9 directly regulates the type-ll collagen gene. Nature Genetics 16, 174-178, doi:10.1038/ng0697-174 (1997).
4 Lefebvre, V., Huang, W. D., Harley, V., Goodfellow, P. N. & de Crombrugghe, B. SOX9 is a potent activator of the chondrocyte-specific enhancer of the Proa1(II) collagen gene. Molecular and cellular biology 17, 2336-2346, doi:10.1128/MCB.17.4.2336 (1997).
5 Bi, W., Deng, J. M., Zhang, Z., Behringer, R. R. & de Crombrugghe, B. Sox9 is required for cartilage formation. Nature Genetics 22, 85-89, doi:10.1038/8792 (1999).
6 Sekiya, I. et al. SOX9 enhances aggrecan gene promoter/enhancer activity and is up-regulated by retinoic acid in a cartilage-derived cell line, TC6. The Journal of biological chemistry 275, 10738-10744 (2000).
7 Liu, C. J. et al. Transcriptional activation of cartilage oligomeric matrix protein by Sox9, Sox5, and Sox6 transcription factors and CBP/p300 coactivators. Frontiers in bioscience: a journal and virtual library 12, 3899-3910, doi:10.2741/2359 (2007).
8 Lefebvre, V. & Dvir-Ginzberg, M. SOX9 and the many facets of its regulation in the chondrocyte lineage. Connect Tissue Res 58, 2-14, doi:10.1080/03008207.2016.1183667 (2017).
9 Chikuda, H. et al. Cyclic GMP-dependent protein kinase II is a molecular switch from proliferation to hypertrophic differentiation of chondrocytes. Genes & development 18, 2418-2429, doi:10.1101/gad.1224204 (2004).
10 Huang, W., Chung, U. I., Kronenberg, H. M. & de Crombrugghe, B. The chondrogenic transcription factor Sox9 is a target of signaling by the parathyroid hormone-related peptide in the growth plate of endochondral bones. Proceedings of the National Academy of Sciences of the United States of America 98, 160-165, doi: 10.1073/pnas.011393998 (2001).
11 Haudenschild, D. R., Chen, J., Pang, N., Lotz, M. K. & D'Lima, D. D. Rho kinase-dependent activation of SOX9 in chondrocytes. Arthritis and rheumatism 62, 191-200, doi:10.1002/art.25051 (2010).
12 Joyce, M. E., Roberts, A. B., Sporn, M. B. & Bolander, M. E. Transforming growth factor-beta and the initiation of chondrogenesis and osteogenesis in the rat femur. The Journal of cell biology 110, 2195-2207 (1990).
13 Kawakami, Y., Rodriguez-León, J. & Izpisúa Belmonte, J. C. The role of TGFbetas and Sox9 during limb chondrogenesis. Current opinion in cell biology 18, 723-729, doi:10.1016/j.ceb.2006.10.007 (2006).
14 Chen, C. G., Thuillier, D., Chin, E. N. & Alliston, T. Chondrocyte-intrinsic Smad3 represses Runx2-inducible matrix metalloproteinase 13 expression to maintain articular cartilage and prevent osteoarthritis. Arthritis and rheumatism 64, 3278-3289, doi:10.1002/art.34566 (2012).
15 Yang, X. et al. TGF-beta/Smad3 signals repress chondrocyte hypertrophic differentiation and are required for maintaining articular cartilage. The Journal of cell biology 153, 35-46 (2001).
16 Coricor, G. & Serra, R. TGF-β regulates phosphorylation and stabilization of Sox9 protein in chondrocytes through p38 and Smad dependent mechanisms. Scientific Reports 6, 38616, doi:10.1038/srep38616 (2016).
17 Chavez, R. D., Coricor, G., Perez, J., Seo, H. S. & Serra, R. SOX9 protein is stabilized by TGF-β and regulates PAPSS2 mRNA expression in chondrocytes. Osteoarthritis and Cartilage 25, 332-340, (2017).
18 Furumatsu, T., Tsuda, M., Taniguchi, N., Tajima, Y. & Asahara, H. Smad3 induces chondrogenesis through the activation of SOX9 via CREB-binding protein/p300 recruitment. The Journal of biological chemistry 280, 8343-8350, doi:10.1074/jbc.M413913200 (2005).
19 Baron, R. & Rawadi, G. Targeting the Wnt/beta-catenin pathway to regulate bone formation in the adult skeleton. Endocrinology 148, 2635-2643, doi:10.1210/en.2007-0270 (2007).
20 Gaur, T. et al. Canonical WNT signaling promotes osteogenesis by directly stimulating Runx2 gene expression. The Journal of biological chemistry 280, 33132-33140, doi:10.1074/jbc.M500608200 (2005).
21 Kugimiya, F. et al. GSK-3beta controls osteogenesis through regulating Runx2 activity. PLoS One 2, e837, doi:10.1371/journal.pone.0000837 (2007).
22 Denker, A. E., Haas, A. R., Nicoll, S. B. & Tuan, R. S. Chondrogenic differentiation of murine C3H10T1/2 multipotential mesenchymal cells: I. Stimulation by bone morphogenetic protein-2 in high-density micromass cultures. Differentiation; research in biological diversity 64, 67-76, doi:10.1046/j.1432-0436.1999.6420067.x (1999).
23 Redondo-Castro, E. et al. Generation of Human Mesenchymal Stem Cell 3D Spheroids Using Low-binding Plates. Bio Protoc 8, doi:10.21769/BioProtoc.2968 (2018).
24 Xu, C., Kim, N. G. & Gumbiner, B. M. Regulation of protein stability by GSK3 mediated phosphorylation. Cell Cycle 8, 4032-4039, doi:10.4161/cc.8.24.10111 (2009).
25 Huang, S. S., Hsu, L. J. & Chang, N. S. Functional role of WW domain-containing proteins in tumor biology and diseases: Insight into the role in ubiquitin-proteasome system. FASEB Bioadv 2, 234-253, doi:10.1096/fba.2019-00060 (2020).
26 Stickens, D. et al. Altered endochondral bone development in matrix metalloproteinase 13-deficient mice. Development 131, 5883, doi:10.1242/dev.01461 (2004).
27 Gulati, G. S. et al. Isolation and functional assessment of mouse skeletal stem cell lineage. Nature Protocols 13, 1294-1309, doi:10.1038/nprot.2018.041 (2018).
28 Ashton, B. A. et al. Formation of bone and cartilage by marrow stromal cells in diffusion chambers in vivo. Clin Orthop Relat Res, 294-307 (1980).
29 Noel, D. et al. Short-term BMP-2 expression is sufficient for in vivo osteochondral differentiation of mesenchymal stem cells. Stem Cells 22, 74-85, doi:10.1634/stemcells.22-1-74 (2004).
30 Lee, J. et al. Fully Dedifferentiated Chondrocytes Expanded in Specific Mesenchymal Stem Cell Growth Medium with FGF2 Obtains Mesenchymal Stem Cell Phenotype In Vitro but Retains Chondrocyte Phenotype In Vivo. Cell Transplant 26, 1673-1687, doi:10.1177/0963689717724794 (2017).
31 Siegel, P. M. & Massague, J. Cytostatic and apoptotic actions of TGF-beta in homeostasis and cancer. Nature reviews. Cancer 3, 807-821, doi:10.1038/nrc1208 (2003).
32 Wu, M., Chen, G. & Li, Y. P. TGF-beta and BMP signaling in osteoblast, skeletal development, and bone formation, homeostasis and disease. Bone research 4, 16009, doi:10.1038/boneres.2016.9 (2016).
33 Matsunobu, T. et al. Critical roles of the TGF-β type I receptor ALK5 in perichondrial formation and function, cartilage integrity, and osteoblast differentiation during growth plate development. Developmental Biology 332, 325-338, (2009).
34 Massague, J., Seoane, J. & Wotton, D. Smad transcription factors. Genes & development 19, 2783-2810, doi:10.1101/gad.1350705 (2005).
35 Kang, J. S., Alliston, T., Delston, R. & Derynck, R. Repression of Runx2 function by TGF-beta through recruitment of class II histone deacetylases by Smad3. The EMBO journal 24, 2543-2555, doi:10.1038/sj.emboj.7600729 (2005).
36 Chen, C. R., Kang, Y., Siegel, P. M. & Massague, J. E2F4/5 and p107 as Smad cofactors linking the TGFbeta receptor to c-myc repression. Cell 110, 19-32 (2002).
37 Minoo, P. et al. SMAD3 prevents binding of NKX2.1 and FOXA1 to the SpB promoter through its MH1 and MH2 domains. Nucleic acids research 36, 179-188, doi:10.1093/nar/gkm871 (2008).
38 Deribe, Y. L., Pawson, T. & Dikic, I. Post-translational modifications in signal integration. Nature structural & molecular biology 17, 666-672, doi:10.1038/nsmb.1842 (2010).
39 Hong, X. et al. SOX9 is targeted for proteasomal degradation by the E3 ligase FBW7 in response to DNA damage. Nucleic acids research 44, 8855-8869, doi:10.1093/nar/gkw748 (2016).
40 Akiyama, H., Chaboissier, M. C., Martin, J. F., Schedl, A. & de Crombrugghe, B. The transcription factor Sox9 has essential roles in successive steps of the chondrocyte differentiation pathway and is required for expression of Sox5 and Sox6. Genes & development 16, 2813-2828, doi:10.1101/gad.1017802 (2002).
41 Hattori, T. et al. E6-AP/UBE3A protein acts as a ubiquitin ligase toward SOX9 protein. The Journal of biological chemistry 288, 35138-35148, doi:10.1074/jbc.M113.486795 (2013).
42 Clevers, H. & Nusse, R. Wnt/beta-catenin signaling and disease. Cell 149, 1192-1205, doi:10.1016/j.cell.2012.05.012 (2012).
43 Sutherland, C. What Are the bona fide GSK3 Substrates? Int J Alzheimers Dis 2011, 505607, doi:10.4061/2011/505607 (2011).
44 Clement-Lacroix, P. et al. Lrp5-independent activation of Wnt signaling by lithium chloride increases bone formation and bone mass in mice. Proceedings of the National Academy of Sciences of the United States of America 102, 17406-17411, doi:10.1073/pnas.0505259102 (2005).
45 Marsell, R. et al. GSK-3 inhibition by an orally active small molecule increases bone mass in rats. Bone 50, 619-627, doi:10.1016/j.bone.2011.11.007 (2012).
46 Pearce, L. R., Komander, D. & Alessi, D. R. The nuts and bolts of AGC protein kinases. Nature reviews. Molecular cell biology 11, 9-22, doi:10.1038/nrm2822 (2010).
47 Clay, M. R., Varma, S. & West, R. B. MAST2 and NOTCH1 translocations in breast carcinoma and associated pre-invasive lesions. Human pathology 44, 2837-2844, doi:10.1016/j.humpath.2013.08.001 (2013).
48 Valiente, M. et al. Binding of PTEN to specific PDZ domains contributes to PTEN protein stability and phosphorylation by microtubule-associated serine/threonine kinases. The Journal of biological chemistry 280, 28936-28943, doi:10.1074/jbc.M504761200 (2005).
49 Hibar, D. P. et al. Novel genetic loci associated with hippocampal volume. Nature communications 8, 13624, doi:10.1038/ncomms13624 (2017).
50 Steffens, M. et al. Genome-wide association analysis of genetic generalized epilepsies implicates susceptibility loci at 1q43, 2p16.1, 2q22.3 and 17q21.32. Human molecular genetics 21, 5359-5372, doi:10.1093/hmg/dds373 (2012).
51 De Simone, R., Puig, X. S., Gelisse, P., Crespel, A. & Genton, P. Senile myoclonic epilepsy: delineation of a common condition associated with Alzheimer's disease in Down syndrome. Seizure 19, 383-389, doi:10.1016/j.seizure.2010.04.008 (2010).
52 Nagel, A. K., Schilling, M., Comte-Walters, S., Berkaw, M. N. & Ball, L. E. Identification of O-linked N-acetylglucosamine (O-GlcNAc)-modified osteoblast proteins by electron transfer dissociation tandem mass spectrometry reveals proteins critical for bone formation. Mol Cell Proteomics 12, 945-955, doi:10.1074/mcp.M112.026633 (2013).
53 Lee, S.-J. et al. Mast4 knockout shows the regulation of spermatogonial stem cell self-renewal via the FGF2/ERM pathway. Cell Death & Differentiation 28, 1441-1454, doi:10.1038/s41418-020-00670-2 (2021).
54 Meka, S. R. K., Chacko, L. A., Ravi, A., Chatterjee, K. & Ananthanarayanan, V. Role of Microtubules in Osteogenic Differentiation of Mesenchymal Stem Cells on 3D Nanofibrous Scaffolds. ACS Biomaterials Science & Engineering 3, 551-559, doi:10.1021/acsbiomaterials.6b00725 (2017).
55 Cong, L. et al. Multiplex genome engineering using CRISPR/Cas systems. Science (New York, N.Y.) 339, 819-823, doi:10.1126/science.1231143 (2013).
56 Yi, T. et al. Manufacture of Clinical-Grade Human Clonal Mesenchymal Stem Cell Products from Single Colony Forming Unit-Derived Colonies Based on the Subfractionation Culturing Method. Tissue Eng Part C-Me 21, 1251-1262, doi:10.1089/ten.tec.2015.0017 (2015).
57 Lee, D. K. et al. Continuous transforming growth factor beta1 secretion by cell-mediated gene therapy maintains chondrocyte redifferentiation. Tissue Eng 11, 310-318, doi:10.1089/ten.2005.11.310 (2005).
58 Kim, D. et al. TopHat2: accurate alignment of transcriptomes in the presence of insertions, deletions and gene fusions. Genome Biol 14, R36, doi:10.1186/gb-2013-14-4-r36 (2013).
59 Trapnell, C. et al. Transcript assembly and quantification by RNA-Seq reveals unannotated transcripts and isoform switching during cell differentiation. Nature Biotechnology 28, 511-515, doi:10.1038/nbt.1621 (2010).
60 Huang da, W., Sherman, B. T. & Lempicki, R. A. Systematic and integrative analysis of large gene lists using DAVID bioinformatics resources. Nat Protoc 4, 44-57, doi:10.1038/nprot.2008.211 (2009).
61 Subramanian, A. et al. Gene set enrichment analysis: a knowledge-based approach for interpreting genome-wide expression profiles. Proceedings of the National Academy of Sciences of the United States of America 102, 15545-15550, doi:10.1073/pnas.0506580102 (2005).
62 Ramskold, D., Wang, E. T., Burge, C. B. & Sandberg, R. An abundance of ubiquitously expressed genes revealed by tissue transcriptome sequence data. PLOS Comput Biol 5, e1000598, doi:10.1371/journal.pcbi.1000598 (2009).
63 Rauch, D. A. et al. The ARF tumor suppressor regulates bone remodeling and osteosarcoma development in mice. PloS one 5, e15755-e15755, doi:10.1371/journal.pone.0015755 (2010).
64 Park, Y. B. et al. Single-stage cell-based cartilage repair in a rabbit model: cell tracking and in vivo chondrogenesis of human umbilical cord blood-derived mesenchymal stem cells and hyaluronic acid hydrogel composite. Osteoarthritis Cartilage 25, 570-580, doi:10.1016/j.joca.2016.10.012 (2017). All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method of generating bone, comprising administering to a subject in need thereof at or near a site of bone defect, where bone is desired to be formed, eukaryotic cells in which expression or activity of Microtubule Associated Serine/Threonine Kinase Family Member 4 (MAST4) protein or a fragment thereof is stabilized or increased compared with normal cell.

2. The method of claim 1, comprising recombinantly expressing MAST4 in the cell.

3. The method of claim 1, wherein the cell is a connective tissue cell.

4. The method of claim 3, wherein the cell is mesenchymal stem cell, fibroblast, osteoprogenitor cell, osteocyte, preosteoblast, osteoblast or osteoclast.

5. The method of claim 1, wherein the cell is allogeneic with respect to host.

6. The method of claim 2, comprising recombinantly overexpressing MAST4 in the cell.

7. The method of claim 2, wherein the recombinantly expressed MAST4 is under control of a viral promoter.

8. The method of claim 7, wherein the viral promoter may be from lentivirus, or adeno-associated virus.

9. The method of claim 1, wherein the cells are contacted with a composition comprising (1) a compound that specifically binds to nucleic acid encoding a MAST4 inhibiting protein thus inhibiting expression of the MAST4 inhibiting protein; or (2) a compound that specifically binds to a MAST4 inhibiting protein thus preventing its binding to MAST4.

10. The method of claim 9, wherein the MAST4 inhibiting protein is GSK-3 family.

11. The method of claim 10, wherein the compound is a chemical, polypeptide, or polynucleotide, or a combination thereof.

12. The method of claim 11, wherein the polypeptide is an antibody or an antigen-binding molecule.

13. The method of claim 11, wherein the compound is microRNA (miRNA), small interfering RNA (siRNA), short hairpin RNA (shRNA), Piwi-interacting RNA (piRNA), small nuclear RNA (snRNA), or antisense oligonucleotide, or a combination thereof.

14. The method of claim 11, wherein the compound is CRISPR-Cas comprising guide RNA specific to the nucleic acid encoding the MAST4 inhibiting protein (or the fragment thereof).

15. The method of claim 14, wherein the guide RNA is a dual RNA comprising CRISPR RNA (crRNA) and transactivating crRNA (tracrRNA) specific to the nucleic acid encoding the MAST4 inhibiting protein (or the fragment thereof), or a single strand guide RNA comprising parts of the crRNA and the tracrRNA and hybridizing with the nucleic acid encoding the MAST4 inhibiting protein (or the fragment thereof).

16. The method of claim 1, wherein the cell is a connective tissue cell.

17. The method of claim 16, wherein the cell is mesenchymal stem cell, fibroblast, osteoprogenitor cell, osteocyte, preosteoblast, osteoblast or osteoclast.

18. The method of claim 9, wherein the cell is allogeneic with respect to host.

19. The method of claim 9, wherein the cell further comprises a recombinant construct that expresses MAST4.

20. The method of claim 19, wherein the recombinant construct overexpresses MAST4.

21. A method of producing extracellular matrix from eukaryotic cells, comprising contacting the eukaryotic cells with a composition comprising a compound capable of specifically binding to a nucleic acid encoding Microtubule Associated Serine/Threonine Kinase Family Member 4 (MAST4) protein or a fragment thereof and inhibits expression or activity of the MAST4 protein, wherein the compound capable of specifically binding to the nucleic acid encoding the MAST4 protein or the fragment thereof, wherein the eukaryotic cells are chondrocytes, fibroblasts or mesenchymal stem cells.

22. The method of claim 21, wherein the compound is microRNA (miRNA), small interfering RNA (siRNA), short hairpin RNA (shRNA), Piwi-interacting RNA (piRNA), small nuclear RNA (snRNA), or antisense oligonucleotide, or a combination thereof.

23. A method of preventing, treating, or improving a joint disease, the method comprising
(i) administering a compound to inhibit Microtubule Associated Serine/Threonine Kinase Family Member 4 (MAST4) in a eukaryotic cell, such that MAST4 protein expression or activity is inhibited; and
(ii) administering to a subject in need thereof at or near a joint in need thereof where cartilage is desired to be formed, the eukaryotic cells obtained thereby.


SEQUENCE LISTING (44 sequences)

SEQ ID NO: 1 - protein, 2434 aa, Homo sapiens
MDESSILRRRGLQKELSLPRRGSLIDSQKWNCLVKRCRTSNRKSLIGNGQSPALPRPHSPLSAHAGNSPQDSPRNFSPSASAHFSFARRTDGRRWSLASLPSSGYGTNTPSSTVSSSCSSQEKLHQLPYQPTPDELHFLSKHFCTTESIATENRCRNTPMRPRSRSLSPGRSPACCDHEIIMMNHVYKERFPKATAQMEERLKEIITSYSPDNVLPLADGVLSFTHHQIIELARDCLDKSHQGLITSRYFLELQHKLDKLLQEAHDRSESGELAFIKQLVRKILIVIARPARLLECLEFDPEEFYYLLEAAEGHAKEGQGIKTDIPRYIISQLGLNKDPLEEMAHLGNYDSGTAETPETDESVSSSNASLKLRRKPRESDFETIKLISNGAYGAVYFVRHKESRQRFAMKKINKQNLILRNQIQQAFVERDILTFAENPFVVSMYCSFETRRHLCMVMEYVEGGDCATLMKNMGPLPVDMARMYFAETVLALEYLHNYGIVHRDLKPDNLLVTSMGHIKLTDFGLSKVGLMSMTTNLYEGHIEKDAREFLDKQVCGTPEYIAPEVILRQGYGKPVDWWAMGIILYEFLVGCVPFFGDTPEELFGQVISDEINWPEKDEAPPPDAQDLITLLLRQNPLERLGTGGAYEVKQHRFFRSLDWNSLLRQKAEFIPQLESEDDTSYFDTRSEKYHHMETEEEDDTNDEDFNVEIRQFSSCSHRFSKVFSSIDRITQNSAEEKEDSVDKTKSTTLPSTETLSWSSEYSEMQQLSTSNSSDTESNRHKLSSGLLPKLAISTEGEQDEAASCPGDPHEEPGKPALPPEECAQEEPEVTTPASTISSSTLSVGSFSEHLDQINGRSECVDSTDNSSKPSSEPASHMARQRLESTEKKKISGKVTKSLSASALSLMIPGDMFAVSPLGSPMSPHSLSSDPSSSRDSSPSRDSSAASASPHQPIVIHSSGKNYGFTIRAIRVYVGDSDIYTVHHIVWNVEEGSPACQAGLKAGDLITHINGEPVHGLVHTEVIELLLKSGNKVSITTTPFENTSIKTGPARRNSYKSRMVRRSKKSKKKESLERRRSLFKKLAKQPSPLLHTSRSFSCLNRSLSSGESLPGSPTHSLSPRSPTPSYRSTPDFPSGTNSSQSSSPSSSAPNSPAGSGHIRPSTLHGLAPKLGGQRYRSGRRKSAGNIPLSPLARTPSPTPQPTSPQRSPSPLLGHSLGNSKIAQAFPSKMHSPPTIVRHIVRPKSAEPPRSPLLKRVQSEEKLSPSYGSDKKHLCSRKHSLEVTQEEVQREQSQREAPLQSLDENVCDVPPLSRARPVEQGCLKRPVSRKVGRQESVDDLDRDKLKAKVVVKKADGFPEKQESHQKSHGPGSDLENFALFKLEEREKKVYPKAVERSSTFENKASMQEAPPLGSLLKDALHKQASVRASEGAMSDGRVPAEHRQGGGDFRRAPAPGTLQDGLCHSLDRGISGKGEGTEKSSQAKELLRCEKLDSKLANIDYLRKKMSLEDKEDNLCPVLKPKMTAGSHECLPGNPVRPTGGQQEPPPASESRAFVSSTHAAQMSAVSFVPLKALTGRVDSGTEKPGLVAPESPVRKSPSEYKLEGRSVSCLKPIEGTLDIALLSGPQASKTELPSPESAQSPSPSGDVRASVPPVLPSSSGKKNDTTSARELSPSSLKMNKSYLLEPWFLPPSRGLQNSPAVSLPDPEFKRDRKGPHPTARSPGTVMESNPQQREGSSPKHQDHTTDPKLLTCLGQNLHSPDLARPRCPLPPEASPSREKPGLRESSERGPPTARSERSAARADTCREPSMELCFPETAKTSDNSKNLLSVGRTHPDFYTQTQAMEKAWAPGGKTNHKDGPGEARPPPRDNSSLHSAGIPCEKELGKVRRGVEPKPEALLARRSLQPPGIESEKSEKLSSFPSLQKDGAKEPERKEQPLQRHPSSIPPPPLTAKDLSSPAARQHCSSPSHASGREPGAKPSTAEPSSSPQDPPKPVAAHSESSSHKPRPGPDPGPPKTKHPDRSLSSQKPSVGATKGKEPATQSLGGSSREGKGHSKSGPDVFPATPGSQNKASDGIGQGEGGPSVPLHTDRAPLDAKPQPTSGGRPLEVLEKPVHLPRPGHPGPSEPADQKLSAVGEKQTLSPKHPKPSTVKDCPTLCKQTDNRQTDKSPSQPAANTDRRAEGKKCTEALYAPAEGDKLEAGLSFVHSENRLKGAERPAAGVGKGFPEARGKGPGPQKPPTEADKPNGMKRSPSATGQSSFRSTALPEKSLSCSSSFPETRAGVREASAASSDTSSAKAAGGMLELPAPSNRDHRKAQPAGEGRTHMTKSDSLPSFRVSTLPLESHHPDPNTMGGASHRDRALSVTATVGETKGKDPAPAQPPPARKQNVGRDVTKPSPAPNTDRPISLSNEKDFVVRQRRGKESLRSSPHKKAL

SEQ ID NO: 2 - protein, 250 aa, Homo sapiens
MGEKVSEAPEPVPRGCSGHGSRTPASALVAASSPGASSAESSSGSETLSEEGEPGGFSREHQPPPPPPLGGTLGARAPAAWAPASVLLERGVLALPPPLPGGAVPPAPRGSSASQEEQDEELDHILSPPPMPFRKCSNPDVASGPGKSLKYKRQLSEDGRQLRRGSLGGALTGRYLLPNPVAGQAWPASAETSNLVRMRSQALGQSAPSLTASLKELSLPRRGSLIDSQKWNCLVKRPVCPNAGRTSPLG

SEQ ID NO: 3 - protein, 2623 aa, Homo sapiens
MGEKVSEAPEPVPRGCSGHGSRTPASALVAASSPGASSAESSSGSETLSEEGEPGGFSREHQPPPPPPLGGTLGARAPAAWAPASVLLERGVLALPPPLPGGAVPPAPRGSSASQEEQDEELDHILSPPPMPFRKCSNPDVASGPGKSLKYKRQLSEDGRQLRRGSLGGALTGRYLLPNPVAGQAWPASAETSNLVRMRSQALGQSAPSLTASLKELSLPRRGSFCRTSNRKSLIGNGQSPALPRPHSPLSAHAGNSPQDSPRNFSPSASAHFSFARRTDGRRWSLASLPSSGYGTNTPSSTVSSSCSSQEKLHQLPYQPTPDELHFLSKHFCTTESIATENRCRNTPMRPRSRSLSPGRSPACCDHEIIMMNHVYKERFPKATAQMEERLKEIITSYSPDNVLPLADGVLSFTHHQIIELARDCLDKSHQGLITSRYFLELQHKLDKLLQEAHDRSESGELAFIKQLVRKILIVIARPARLLECLEFDPEEFYYLLEAAEGHAKEGQGIKTDIPRYIISQLGLNKDPLEEMAHLGNYDSGTAETPETDESVSSSNASLKLRRKPRESDFETIKLISNGAYGAVYFVRHKESRQRFAMKKINKQNLILRNQIQQAFVERDILTFAENPFVVSMYCSFETRRHLCMVMEYVEGGDCATLMKNMGPLPVDMARMYFAETVLALEYLHNYGIVHRDLKPDNLLVTSMGHIKLTDFGLSKVGLMSMTTNLYEGHIEKDAREFLDKQVCGTPEYIAPEVILRQGYGKPVDWWAMGIILYEFLVGCVPFFGDTPEELFGQVISDEINWPEKDEAPPPDAQDLITLLLRQNPLERLGTGGAYEVKQHRFFRSLDWNSLLRQKAEFIPQLESEDDTSYFDTRSEKYHHMETEEEDDTNDEDFNVEIRQFSSCSHRFSKVFSSIDRITQNSAEEKEDSVDKTKSTTLPSTETLSWSSEYSEMQQLSTSNSSDTESNRHKLSSGLLPKLAISTEGEQDEAASCPGDPHEEPGKPALPPEECAQEEPEVTTPASTISSSTLSVGSFSEHLDQINGRSECVDSTDNSSKPSSEPASHMARQRLESTEKKKISGKVTKSLSASALSLMIPGDMFAVSPLGSPMSPHSLSSDPSSSRDSSPSRDSSAASASPHQPIVIHSSGKNYGFTIRAIRVYVGDSDIYTVHHIVWNVEEGSPACQAGLKAGDLITHINGEPVHGLVHTEVIELLLKSGNKVSITTTPFENTSIKTGPARRNSYKSRMVRRSKKSKKKESLERRRSLFKKLAKQPSPLLHTSRSFSCLNRSLSSGESLPGSPTHSLSPRSPTPSYRSTPDFPSGTNSSQSSSPSSSAPNSPAGSGHIRPSTLHGLAPKLGGQRYRSGRRKSAGNIPLSPLARTPSPTPQPTSPQRSPSPLLGHSLGNSKIAQAFPSKMHSPPTIVRHIVRPKSAEPPRSPLLKRVQSEEKLSPSYGSDKKHLCSRKHSLEVTQEEVQREQSQREAPLQSLDENVCDVPPLSRARPVEQGCLKRPVSRKVGRQESVDDLDRDKLKAKVVVKKADGFPEKQESHQKSHGPGSDLENFALFKLEEREKKVYPKAVERSSTFENKASMQEAPPLGSLLKDALHKQASVRASEGAMSDGRVPAEHRQGGGDFRRAPAPGTLQDGLCHSLDRGISGKGEGTEKSSQAKELLRCEKLDSKLANIDYLRKKMSLEDKEDNLCPVLKPKMTAGSHECLPGNPVRPTGGQQEPPPASESRAFVSSTHAAQMSAVSFVPLKALTGRVDSGTEKPGLVAPESPVRKSPSEYKLEGRSVSCLKPIEGTLDIALLSGPQASKTELPSPESAQSPSPSGDVRASVPPVLPSSSGKKNDTTSARELSPSSLKMNKSYLLEPWFLPPSRGLQNSPAVSLPDPEFKRDRKGPHPTARSPGTVMESNPQQREGSSPKHQDHTTDPKLLTCLGQNLHSPDLARPRCPLPPEASPSREKPGLRESSERGPPTARSERSAARADTCREPSMELCFPETAKTSDNSKNLLSVGRTHPDFYTQTQAMEKAWAPGGKTNHKDGPGEARPPPRDNSSLHSAGIPCEKELGKVRRGVEPKPEALLARRSLQPPGIESEKSEKLSSFPSLQKDGAKEPERKEQPLQRHPSSIPPPPLTAKDLSSPAARQHCSSPSHASGREPGAKPSTAEPSSSPQDPPKPVAAHSESSSHKPRPGPDPGPPKTKHPDRSLSSQKPSVGATKGKEPATQSLGGSSREGKGHSKSGPDVFPATPGSQNKASDGIGQGEGGPSVPLHTDRAPLDAKPQPTSGGRPLEVLEKPVHLPRPGHPGPSEPADQKLSAVGEKQTLSPKHPKPSTVKDCPTLCKQTDNRQTDKSPSQPAANTDRRAEGKKCTEALYAPAEGDKLEAGLSFVHSENRLKGAERPAAGVGKGFPEARGKGPGPQKPPTEADKPNGMKRSPSATGQSSFRSTALPEKSLSCSSSFPETRAGVREASAASSDTSSAKAAGGMLELPAPSNRDHRKAQPAGEGRTHMTKSDSLPSFRVSTLPLESHHPDPNTMGGASHRDRALSVTATVGETKGKDPAPAQPPPARKQNVGRDVTKPSPAPNTDRPISLSNEKDFVVRQRRGKESLRSSPHKKAL

SEQ ID NO: 4 - protein, 2417 aa, Homo sapiens
MKAQRERLQIPGLTLDCRTSNRKSLIGNGQSPALPRPHSPLSAHAGNSPQDSPRNFSPSASAHFSFARRNDRTDGRRWSLASLPSSGYGTNTPSSTVSSSCSSQEKLHQLPYQPTPDELHFLSKHFCTTESIATENRCRNTPMRPRSRSLSPGRSPACCDHEIIMMNHVYKERFPKATAQMEERLKEIITSYSPDNVLPLADGVLSFTHHQIIELARDCLDKSHQGLITSRYFLELQHKLDKLLQEAHDRSESGELAFIKQLVRKILIVIARPARLLECLEFDPEEFYYLLEAAEGHAKEGQGIKTDIPRYIISQLGLNKDPLEEMAHLGNYDSGTAETPETDESVSSSNASLKLRRKPRESDFETIKLISNGAYGAVYFVRHKESRQRFAMKKINKQNLILRNQIQQAFVERDILTFAENPFVVSMYCSFETRRHLCMVMEYVEGGDCATLMKNMGPLPVDMARMYFAETVLALEYLHNYGIVHRDLKPDNLLVTSMGHIKLTDFGLSKVGLMSMTTNLYEGHIEKDAREFLDKQVCGTPEYIAPEVILRQGYGKPVDWWAMGIILYEFLVGCVPFFGDTPEELFGQVISDEINWPEKDEAPPPDAQDLITLLLRQNPLERLGTGGAYEVKQHRFFRSLDWNSLLRQKAEFIPQLESEDDTSYFDTRSEKYHHMETEEEDDTNDEDFNVEIRQFSSCSHRFSKVFSSIDRITQNSAEEKEDSVDKTKSTTLPSTETLSWSSEYSEMQQLSTSNSSDTESNRHKLSSGLLPKLAISTEGEQDEAASCPGDPHEEPGKPALPPEECAQEEPEVTTPASTISSSTLSVGSFSEHLDQINGRSECVDSTDNSSKPSSEPASHMARQRLESTEKKKISGKVTKSLSASALSLMIPGDMFAVSPLGSPMSPHSLSSDPSSSRDSSPSRDSSAASASPHQPIVIHSSGKNYGFTIRAIRVYVGDSDIYTVHHIVWNVEEGSPACQAGLKAGDLITHINGEPVHGLVHTEVIELLLKSGNKVSITTTPFENTSIKTGPARRNSYKSRMVRRSKKSKKKESLERRRSLFKKLAKQPSPLLHTSRSFSCLNRSLSSGESLPGSPTHSLSPRSPTPSYRSTPDFPSGTNSSQSSSPSSSAPNSPAGSGHIRPSTLHGLAPKLGGQRYRSGRRKSAGNIPLSPLARTPSPTPQPTSPQRSPSPLLGHSLGNSKIAQAFPSKMHSPPTIVRHIVRPKSAEPPRSPLLKRVQSEEKLSPSYGSDKKHLCSRKHSLEVTQEEVQREQSQREAPLQSLDENVCDVPPLSRARPVEQGCLKRPVSRKVGRQESVDDLDRDKLKAKVVVKKADGFPEKQESHQKSHGPGSDLENFALFKLEEREKKVYPKAVERSSTFENKASMQEAPPLGSLLKDALHKQASVRASEGAMSDGRVPAEHRQGGGDFRRAPAPGTLQDGLCHSLDRGISGKGEGTEKSSQAKELLRCEKLDSKLANIDYLRKKMSLEDKEDNLCPVLKPKMTAGSHECLPGNPVRPTGGQQEPPPASESRAFVSSTHAAQMSAVSFVPLKALTGRVDSGTEKPGLVAPESPVRKSPSEYKLEGRSVSCLKPIEGTLDIALLSGPQASKTELPSPESAQSPSPSGDVRASVPPVLPSSSGKKNDTTSARELSPSSLKMNKSYLLEPWFLPPSRGLQNSPAVSLPDPEFKRDRKGPHPTARSPGTVMESNPQQREGSSPKHQDHTTDPKLLTCLGQNLHSPDLARPRCPLPPEASPSREKPGLRESSERGPPTARSERSAARADTCREPSMELCFPETAKTSDNSKNLLSVGRTHPDFYTQTQAMEKAWAPGGKTNHKDGPGEARPPPRDNSSLHSAGIPCEKELGKVRRGVEPKPEALLARRSLQPPGIESEKSEKLSSFPSLQKDGAKEPERKEQPLQRHPSSIPPPPLTAKDLSSPAARQHCSSPSHASGREPGAKPSTAEPSSSPQDPPKPVAAHSESSSHKPRPGPDPGPPKTKHPDRSLSSQKPSVGATKGKEPATQSLGGSSREGKGHSKSGPDVFPATPGSQNKASDGIGQGEGGPSVPLHTDRAPLDAKPQPTSGGRPLEVLEKPVHLPRPGHPGPSEPADQKLSAVGEKQTLSPKHPKPSTVKDCPTLCKQTDNRQTDKSPSQPAANTDRRAEGKKCTEALYAPAEGDKLEAGLSFVHSENRLKGAERPAAGVGKGFPEARGKGPGPQKPPTEADKPNGMKRSPSATGQSSFRSTALPEKSLSCSSSFPETRAGVREASAASSDTSSAKAAGGMLELPAPSNRDHRKAQPAGEGRTHMTKSDSLPSFRVSTLPLESHHPDPNTMGGASHRDRALSVTATVGETKGKDPAPAQPPPARKQNVGRDVTKPSPAPNTDRPISLSNEKDFVVRQRRGKESLRSSPHKKAL

SEQ ID NO: 5 - protein, 2362 aa, Homo sapiens
MDMSDPNFWTVLSNFTLPHLRSGNRLRRTQSCRTSNRKSLIGNGQSPALPRPHSPLSAHAGNSPQDSPRNFSPSASAHFSFARRTDGRRWSLASLPSSGYGTNTPSSTVSSSCSSQEKLHQLPYQPTPDELHFLSKHFCTTESIATENRCRNTPMRPRSRSLSPGRSPACCDHEIIMMNHVYKERFPKATAQMEERLKEIITSYSPDNVLPLADGVLSFTHHQIIELARDCLDKSHQGLITSRYFLELQHKLDKLLQEAHDRSESGELAFIKQLVRKILIVIARPARLLECLEFDPEEFYYLLEAAEGHAKEGQGIKTDIPRYIISQLGLNKDPLEEMAHLGNYDSGTAETPETDESVSSSNASLKLRRKPRESDFETIKLISNGAYGAVYFVRHKESRQRFAMKKINKQNLILRNQIQQAFVERDILTFAENPFVVSMYCSFETRRHLCMVMEYVEGGDCATLMKNMGPLPVDMARMYFAETVLALEYLHNYGIVHRDLKPDNLLVTSMGHIKLTDFGLSKVGLMSMTTNLYEGHIEKDAREFLDKQVCGTPEYIAPEVILRQGYGKPVDWWAMGIILYEFLVGCVPFFGDTPEELFGQVISDEINWPEKDEAPPPDAQDLITLLLRQNPLERLGTGGAYEVKQHRFFRSLDWNSLLRQKAEFIPQLESEDDTSYFDTRSEKYHHMETEEEDDTNDEDFNVEIRQFSSCSHRFSKVFSSIDRITQNSAEEKEDSVDKTKSTTLPSTETLSWSSEYSEMQQLSTSNSSDTESNRHKLSSGLLPKLAISTEGEQDEAASCPGDPHEEPGKPALPPEECAQEEPEVTTPASTISSSTLSDMFAVSPLGSPMSPHSLSSDPSSSRDSSPSRDSSAASASPHQPIVIHSSGKNYGFTIRAIRVYVGDSDIYTVHHIVWNVEEGSPACQAGLKAGDLITHINGEPVHGLVHTEVIELLLKSGNKVSITTTPFENTSIKTGPARRNSYKSRMVRRSKKSKKKESLERRRSLFKKLAKQPSPLLHTSRSFSCLNRSLSSGESLPGSPTHSLSPRSPTPSYRSTPDFPSGTNSSQSSSPSSSAPNSPAGSGHIRPSTLHGLAPKLGGQRYRSGRRKSAGNIPLSPLARTPSPTPQPTSPQRSPSPLLGHSLGNSKIAQAFPSKMHSPPTIVRHIVRPKSAEPPRSPLLKRVQSEEKLSPSYGSDKKHLCSRKHSLEVTQEEVQREQSQREAPLQSLDENVCDVPPLSRARPVEQGCLKRPVSRKVGRQESVDDLDRDKLKAKVVVKKADGFPEKQESHQKSHGPGSDLENFALFKLEEREKKVYPKAVERSSTFENKASMQEAPPLGSLLKDALHKQASVRASEGAMSDGRVPAEHRQGGGDFRRAPAPGTLQDGLCHSLDRGISGKGEGTEKSSQAKELLRCEKLDSKLANIDYLRKKMSLEDKEDNLCPVLKPKMTAGSHECLPGNPVRPTGGQQEPPPASESRAFVSSTHAAQMSAVSFVPLKALTGRVDSGTEKPGLVAPESPVRKSPSEYKLEGRSVSCLKPIEGTLDIALLSGPQASKTELPSPESAQSPSPSGDVRASVPPVLPSSSGKKNDTTSARELSPSSLKMNKSYLLEPWFLPPSRGLQNSPAVSLPDPEFKRDRKGPHPTARSPGTVMESNPQQREGSSPKHQDHTTDPKLLTCLGQNLHSPDLARPRCPLPPEASPSREKPGLRESSERGPPTARSERSAARADTCREPSMELCFPETAKTSDNSKNLLSVGRTHPDFYTQTQAMEKAWAPGGKTNHKDGPGEARPPPRDNSSLHSAGIPCEKELGKVRRGVEPKPEALLARRSLQPPGIESEKSEKLSSFPSLQKDGAKEPERKEQPLQRHPSSIPPPPLTAKDLSSPAARQHCSSPSHASGREPGAKPSTAEPSSSPQDPPKPVAAHSESSSHKPRPGPDPGPPKTKHPDRSLSSQKPSVGATKGKEPATQSLGGSSREGKGHSKSGPDVFPATPGSQNKASDGIGQGEGGPSVPLHTDRAPLDAKPQPTSGGRPLEVLEKPVHLPRPGHPGPSEPADQKLSAVGEKQTLSPKHPKPSTVKDCPTLCKQTDNRQTDKSPSQPAANTDRRAEGKKCTEALYAPAEGDKLEAGLSFVHSENRLKGAERPAAGVGKGFPEARGKGPGPQKPPTEADKPNGMKRSPSATGQSSFRSTALPEKSLSCSSSFPETRAGVREASAASSDTSSAKAAGGMLELPAPSNRDHRKAQPAGEGRTHMTKSDSLPSFRVSTLPLESHHPDPNTMGGASHRDRALSVTATVGETKGKDPAPAQPPPARKQNVGRDVTKPSPAPNTDRPISLSNEKDFVVRQRRGKESLRSSPHKKAL

SEQ ID NO: 6 - protein, 238 aa, Homo sapiens
MGEKVSEAPEPVPRGCSGHGSRTPASALVAASSPGASSAESSSGSETLSEEGEPGGFSREHQPPPPPPLGGTLGARAPAAWAPASVLLERGVLALPPPLPGGAVPPAPRGSSASQEEQDEELDHILSPPPMPFRKCSNPDVASGPGKSLKYKRQLSEDGRQLRRGSLGGALTGRYLLPNPVAGQAWPASAETSNLVRMRSQALGQSAPSLTASLKELSLPRRGSFPVCPNAGRTSPLG

SEQ ID NO: 7 - protein, 2429 aa, Homo sapiens
MDMSDPNFWTVLSNFTLPHLRSGNRLRRTQSCRTSNRKSLIGNGQSPALPRPHSPLSAHAGNSPQDSPRNFSPSASAHFSFARRTDGRRWSLASLPSSGYGTNTPSSTVSSSCSSQEKLHQLPYQPTPDELHFLSKHFCTTESIATENRCRNTPMRPRSRSLSPGRSPACCDHEIIMMNHVYKERFPKATAQMEERLKEIITSYSPDNVLPLADGVLSFTHHQIIELARDCLDKSHQGLITSRYFLELQHKLDKLLQEAHDRSESGELAFIKQLVRKILIVIARPARLLECLEFDPEEFYYLLEAAEGHAKEGQGIKTDIPRYIISQLGLNKDPLEEMAHLGNYDSGTAETPETDESVSSSNASLKLRRKPRESDFETIKLISNGAYGAVYFVRHKESRQRFAMKKINKQNLILRNQIQQAFVERDILTFAENPFVVSMYCSFETRRHLCMVMEYVEGGDCATLMKNMGPLPVDMARMYFAETVLALEYLHNYGIVHRDLKPDNLLVTSMGHIKLTDFGLSKVGLMSMTTNLYEGHIEKDAREFLDKQVCGTPEYIAPEVILRQGYGKPVDWWAMGIILYEFLVGCVPFFGDTPEELFGQVISDEINWPEKDEAPPPDAQDLITLLLRQNPLERLGTGGAYEVKQHRFFRSLDWNSLLRQKAEFIPQLESEDDTSYFDTRSEKYHHMETEEEDDTNDEDFNVEIRQFSSCSHRFSKVFSSIDRITQNSAEEKEDSVDKTKSTTLPSTETLSWSSEYSEMQQLSTSNSSDTESNRHKLSSGLLPKLAISTEGEQDEAASCPGDPHEEPGKPALPPEECAQEEPEVTTPASTISSSTLSVGSFSEHLDQINGRSECVDSTDNSSKPSSEPASHMARQRLESTEKKKISGKVTKSLSASALSLMIPGDMFAVSPLGSPMSPHSLSSDPSSSRDSSPSRDSSAASASPHQPIVIHSSGKNYGFTIRAIRVYVGDSDIYTVHHIVWNVEEGSPACQAGLKAGDLITHINGEPVHGLVHTEVIELLLKSGNKVSITTTPFENTSIKTGPARRNSYKSRMVRRSKKSKKKESLERRRSLFKKLAKQPSPLLHTSRSFSCLNRSLSSGESLPGSPTHSLSPRSPTPSYRSTPDFPSGTNSSQSSSPSSSAPNSPAGSGHIRPSTLHGLAPKLGGQRYRSGRRKSAGNIPLSPLARTPSPTPQPTSPQRSPSPLLGHSLGNSKIAQAFPSKMHSPPTIVRHIVRPKSAEPPRSPLLKRVQSEEKLSPSYGSDKKHLCSRKHSLEVTQEEVQREQSQREAPLQSLDENVCDVPPLSRARPVEQGCLKRPVSRKVGRQESVDDLDRDKLKAKVVVKKADGFPEKQESHQKSHGPGSDLENFALFKLEEREKKVYPKAVERSSTFENKASMQEAPPLGSLLKDALHKQASVRASEGAMSDGRVPAEHRQGGGDFRRAPAPGTLQDGLCHSLDRGISGKGEGTEKSSQAKELLRCEKLDSKLANIDYLRKKMSLEDKEDNLCPVLKPKMTAGSHECLPGNPVRPTGGQQEPPPASESRAFVSSTHAAQMSAVSFVPLKALTGRVDSGTEKPGLVAPESPVRKSPSEYKLEGRSVSCLKPIEGTLDIALLSGPQASKTELPSPESAQSPSPSGDVRASVPPVLPSSSGKKNDTTSARELSPSSLKMNKSYLLEPWFLPPSRGLQNSPAVSLPDPEFKRDRKGPHPTARSPGTVMESNPQQREGSSPKHQDHTTDPKLLTCLGQNLHSPDLARPRCPLPPEASPSREKPGLRESSERGPPTARSERSAARADTCREPSMELCFPETAKTSDNSKNLLSVGRTHPDFYTQTQAMEKAWAPGGKTNHKDGPGEARPPPRDNSSLHSAGIPCEKELGKVRRGVEPKPEALLARRSLQPPGIESEKSEKLSSFPSLQKDGAKEPERKEQPLQRHPSSIPPPPLTAKDLSSPAARQHCSSPSHASGREPGAKPSTAEPSSSPQDPPKPVAAHSESSSHKPRPGPDPGPPKTKHPDRSLSSQKPSVGATKGKEPATQSLGGSSREGKGHSKSGPDVFPATPGSQNKASDGIGQGEGGPSVPLHTDRAPLDAKPQPTSGGRPLEVLEKPVHLPRPGHPGPSEPADQKLSAVGEKQTLSPKHPKPSTVKDCPTLCKQTDNRQTDKSPSQPAANTDRRAEGKKCTEALYAPAEGDKLEAGLSFVHSENRLKGAERPAAGVGKGFPEARGKGPGPQKPPTEADKPNGMKRSPSATGQSSFRSTALPEKSLSCSSSFPETRAGVREASAASSDTSSAKAAGGMLELPAPSNRDHRKAQPAGEGRTHMTKSDSLPSFRVSTLPLESHHPDPNTMGGASHRDRALSVTATVGETKGKDPAPAQPPPARKQNVGRDVTKPSPAPNTDRPISLSNEKDFVVRQRRGKESLRSSPHKKAL

SEQ ID NO: 8 - protein, 2626 aa, Homo sapiens
MGEKVSEAPEPVPRGCSGHGSRTPASALVAASSPGASSAESSSGSETLSEEGEPGGFSREHQPPPPPPLGGTLGARAPAAWAPASVLLERGVLALPPPLPGGAVPPAPRGSSASQEEQDEELDHILSPPPMPFRKCSNPDVASGPGKSLKYKRQLSEDGRQLRRGSLGGALTGRYLLPNPVAGQAWPASAETSNLVRMRSQALGQSAPSLTASLKELSLPRRGSFCRTSNRKSLIGNGQSPALPRPHSPLSAHAGNSPQDSPRNFSPSASAHFSFARRNDRTDGRRWSLASLPSSGYGTNTPSSTVSSSCSSQEKLHQLPYQPTPDELHFLSKHFCTTESIATENRCRNTPMRPRSRSLSPGRSPACCDHEIIMMNHVYKERFPKATAQMEERLKEIITSYSPDNVLPLADGVLSFTHHQIIELARDCLDKSHQGLITSRYFLELQHKLDKLLQEAHDRSESGELAFIKQLVRKILIVIARPARLLECLEFDPEEFYYLLEAAEGHAKEGQGIKTDIPRYIISQLGLNKDPLEEMAHLGNYDSGTAETPETDESVSSSNASLKLRRKPRESDFETIKLISNGAYGAVYFVRHKESRQRFAMKKINKQNLILRNQIQQAFVERDILTFAENPFVVSMYCSFETRRHLCMVMEYVEGGDCATLMKNMGPLPVDMARMYFAETVLALEYLHNYGIVHRDLKPDNLLVTSMGHIKLTDFGLSKVGLMSMTTNLYEGHIEKDAREFLDKQVCGTPEYIAPEVILRQGYGKPVDWWAMGIILYEFLVGCVPFFGDTPEELFGQVISDEINWPEKDEAPPPDAQDLITLLLRQNPLERLGTGGAYEVKQHRFFRSLDWNSLLRQKAEFIPQLESEDDTSYFDTRSEKYHHMETEEEDDTNDEDFNVEIRQFSSCSHRFSKVFSSIDRITQNSAEEKEDSVDKTKSTTLPSTETLSWSSEYSEMQQLSTSNSSDTESNRHKLSSGLLPKLAISTEGEQDEAASCPGDPHEEPGKPALPPEECAQEEPEVTTPASTISSSTLSVGSFSEHLDQINGRSECVDSTDNSSKPSSEPASHMARQRLESTEKKKISGKVTKSLSASALSLMIPGDMFAVSPLGSPMSPHSLSSDPSSSRDSSPSRDSSAASASPHQPIVIHSSGKNYGFTIRAIRVYVGDSDIYTVHHIVWNVEEGSPACQAGLKAGDLITHINGEPVHGLVHTEVIELLLKSGNKVSITTTPFENTSIKTGPARRNSYKSRMVRRSKKSKKKESLERRRSLFKKLAKQPSPLLHTSRSFSCLNRSLSSGESLPGSPTHSLSPRSPTPSYRSTPDFPSGTNSSQSSSPSSSAPNSPAGSGHIRPSTLHGLAPKLGGQRYRSGRRKSAGNIPLSPLARTPSPTPQPTSPQRSPSPLLGHSLGNSKIAQAFPSKMHSPPTIVRHIVRPKSAEPPRSPLLKRVQSEEKLSPSYGSDKKHLCSRKHSLEVTQEEVQREQSQREAPLQSLDENVCDVPPLSRARPVEQGCLKRPVSRKVGRQESVDDLDRDKLKAKVVVKKADGFPEKQESHQKSHGPGSDLENFALFKLEEREKKVYPKAVERSSTFENKASMQEAPPLGSLLKDALHKQASVRASEGAMSDGRVPAEHRQGGGDFRRAPAPGTLQDGLCHSLDRGISGKGEGTEKSSQAKELLRCEKLDSKLANIDYLRKKMSLEDKEDNLCPVLKPKMTAGSHECLPGNPVRPTGGQQEPPPASESRAFVSSTHAAQMSAVSFVPLKALTGRVDSGTEKPGLVAPESPVRKSPSEYKLEGRSVSCLKPIEGTLDIALLSGPQASKTELPSPESAQSPSPSGDVRASVPPVLPSSSGKKNDTTSARELSPSSLKMNKSYLLEPWFLPPSRGLQNSPAVSLPDPEFKRDRKGPHPTARSPGTVMESNPQQREGSSPKHQDHTTDPKLLTCLGQNLHSPDLARPRCPLPPEASPSREKPGLRESSERGPPTARSERSAARADTCREPSMELCFPETAKTSDNSKNLLSVGRTHPDFYTQTQAMEKAWAPGGKTNHKDGPGEARPPPRDNSSLHSAGIPCEKELGKVRRGVEPKPEALLARRSLQPPGIESEKSEKLSSFPSLQKDGAKEPERKEQPLQRHPSSIPPPPLTAKDLSSPAARQHCSSPSHASGREPGAKPSTAEPSSSPQDPPKPVAAHSESSSHKPRPGPDPGPPKTKHPDRSLSSQKPSVGATKGKEPATQSLGGSSREGKGHSKSGPDVFPATPGSQNKASDGIGQGEGGPSVPLHTDRAPLDAKPQPTSGGRPLEVLEKPVHLPRPGHPGPSEPADQKLSAVGEKQTLSPKHPKPSTVKDCPTLCKQTDNRQTDKSPSQPAANTDRRAEGKKCTEALYAPAEGDKLEAGLSFVHSENRLKGAERPAAGVGKGFPEARGKGPGPQKPPTEADKPNGMKRSPSATGQSSFRSTALPEKSLSCSSSFPETRAGVREASAASSDTSSAKAAGGMLELPAPSNRDHRKAQPAGEGRTHMTKSDSLPSFRVSTLPLESHHPDPNTMGGASHRDRALSVTATVGETKGKDPAPAQPPPARKQNVGRDVTKPSPAPNTDRPISLSNEKDFVVRQRRGKESLRSSPHKKAL

SEQ ID NO: 9 - protein, 2556 aa, Homo sapiens
MGEKVSEAPEPVPRGCSGHGSRTPASALVAASSPGASSAESSSGSETLSEEGEPGGFSREHQPPPPPPLGGTLGARAPAAWAPASVLLERGVLALPPPLPGGAVPPAPRGSSASQEEQDEELDHILSPPPMPFRKCSNPDVASGPGKSLKYKRQLSEDGRQLRRGSLGGALTGRYLLPNPVAGQAWPASAETSNLVRMRSQALGQSAPSLTASLKELSLPRRGSFCRTSNRKSLIGNGQSPALPRPHSPLSAHAGNSPQDSPRNFSPSASAHFSFARRTDGRRWSLASLPSSGYGTNTPSSTVSSSCSSQEKLHQLPYQPTPDELHFLSKHFCTTESIATENRCRNTPMRPRSRSLSPGRSPACCDHEIIMMNHVYKERFPKATAQMEERLKEIITSYSPDNVLPLADGVLSFTHHQIIELARDCLDKSHQGLITSRYFLELQHKLDKLLQEAHDRSESGELAFIKQLVRKILIVIARPARLLECLEFDPEEFYYLLEAAEGHAKEGQGIKTDIPRYIISQLGLNKDPLEEMAHLGNYDSGTAETPETDESVSSSNASLKLRRKPRESDFETIKLISNGAYGAVYFVRHKESRQRFAMKKINKQNLILRNQIQQAFVERDILTFAENPFVVSMYCSFETRRHLCMVMEYVEGGDCATLMKNMGPLPVDMARMYFAETVLALEYLHNYGIVHRDLKPDNLLVTSMGHIKLTDFGLSKVGLMSMTTNLYEGHIEKDAREFLDKQVCGTPEYIAPEVILRQGYGKPVDWWAMGIILYEFLVGCVPFFGDTPEELFGQVISDEINWPEKDEAPPPDAQDLITLLLRQNPLERLGTGGAYEVKQHRFFRSLDWNSLLRQKAEFIPQLESEDDTSYFDTRSEKYHHMETEEEDDTNDEDFNVEIRQFSSCSHRFSKVFSSIDRITQNSAEEKEDSVDKTKSTTLPSTETLSWSSEYSEMQQLSTSNSSDTESNRHKLSSGLLPKLAISTEGEQDEAASCPGDPHEEPGKPALPPEECAQEEPEVTTPASTISSSTLSDMFAVSPLGSPMSPHSLSSDPSSSRDSSPSRDSSAASASPHQPIVIHSSGKNYGFTIRAIRVYVGDSDIYTVHHIVWNVEEGSPACQAGLKAGDLITHINGEPVHGLVHTEVIELLLKSGNKVSITTTPFENTSIKTGPARRNSYKSRMVRRSKKSKKKESLERRRSLFKKLAKQPSPLLHTSRSFSCLNRSLSSGESLPGSPTHSLSPRSPTPSYRSTPDFPSGTNSSQSSSPSSSAPNSPAGSGHIRPSTLHGLAPKLGGQRYRSGRRKSAGNIPLSPLARTPSPTPQPTSPQRSPSPLLGHSLGNSKIAQAFPSKMHSPPTIVRHIVRPKSAEPPRSPLLKRVQSEEKLSPSYGSDKKHLCSRKHSLEVTQEEVQREQSQREAPLQSLDENVCDVPPLSRARPVEQGCLKRPVSRKVGRQESVDDLDRDKLKAKVVVKKADGFPEKQESHQKSHGPGSDLENFALFKLEEREKKVYPKAVERSSTFENKASMQEAPPLGSLLKDALHKQASVRASEGAMSDGRVPAEHRQGGGDFRRAPAPGTLQDGLCHSLDRGISGKGEGTEKSSQAKELLRCEKLDSKLANIDYLRKKMSLEDKEDNLCPVLKPKMTAGSHECLPGNPVRPTGGQQEPPPASESRAFVSSTHAAQMSAVSFVPLKALTGRVDSGTEKPGLVAPESPVRKSPSEYKLEGRSVSCLKPIEGTLDIALLSGPQASKTELPSPESAQSPSPSGDVRASVPPVLPSSSGKKNDTTSARELSPSSLKMNKSYLLEPWFLPPSRGLQNSPAVSLPDPEFKRDRKGPHPTARSPGTVMESNPQQREGSSPKHQDHTTDPKLLTCLGQNLHSPDLARPRCPLPPEASPSREKPGLRESSERGPPTARSERSAARADTCREPSMELCFPETAKTSDNSKNLLSVGRTHPDFYTQTQAMEKAWAPGGKTNHKDGPGEARPPPRDNSSLHSAGIPCEKELGKVRRGVEPKPEALLARRSLQPPGIESEKSEKLSSFPSLQKDGAKEPERKEQPLQRHPSSIPPPPLTAKDLSSPAARQHCSSPSHASGREPGAKPSTAEPSSSPQDPPKPVAAHSESSSHKPRPGPDPGPPKTKHPDRSLSSQKPSVGATKGKEPATQSLGGSSREGKGHSKSGPDVFPATPGSQNKASDGIGQGEGGPSVPLHTDRAPLDAKPQPTSGGRPLEVLEKPVHLPRPGHPGPSEPADQKLSAVGEKQTLSPKHPKPSTVKDCPTLCKQTDNRQTDKSPSQPAANTDRRAEGKKCTEALYAPAEGDKLEAGLSFVHSENRLKGAERPAAGVGKGFPEARGKGPGPQKPPTEADKPNGMKRSPSATGQSSFRSTALPEKSLSCSSSFPETRAGVREASAASSDTSSAKAAGGMLELPAPSNRDHRKAQPAGEGRTHMTKSDSLPSFRVSTLPLESHHPDPNTMGGASHRDRALSVTATVGETKGKDPAPAQPPPARKQNVGRDVTKPSPAPNTDRPISLSNEKDFVVRQRRGKESLRSSPHKKAL

SEQ ID NO: 10 - protein, 2365 aa, Homo sapiens
MDESSILRRRGLQKELSLPRRGSLIDSQKWNCLVKRCRTSNRKSLIGNGQSPALPRPHSPLSAHAGNSPQDSPRNFSPSASAHFSFARRTDGRRWSLASLPSSGYGTNTPSSTVSSSCSSQEKLHQLPYQPTPDELHFLSKHFCTTESIATENRCRNTPMRPRSRSLSPGRSPACCDHEIIMMNHVYKERFPKATAQMEERLKEIITSYSPDNVLPLADGVLSFTHHQIIELARDCLDKSHQGLITSRYFLELQHKLDKLLQEAHDRSESGELAFIKQLVRKILIVIARPARLLECLEFDPEEFYYLLEAAEGHAKEGQGIKTDIPRYIISQLGLNKDPLEEMAHLGNYDSGTAETPETDESVSSSNASLKLRRKPRESDFETIKLISNGAYGAVYFVRHKESRQRFAMKKINKQNLILRNQIQQAFVERDILTFAENPFVVSMYCSFETRRHLCMVMEYVEGGDCATLMKNMGPLPVDMARMYFAETVLALEYLHNYGIVHRDLKPDNLLVTSMGHIKLTDFGLSKVGLMSMTTNLYEGHIEKDAREFLDKQVCGTPEYIAPEVILRQGYGKPVDWWAMGIILYEFLVGCVPFFGDTPEELFGQVISDEINWPEKDEAPPPDAQDLITLLLRQNPLERLGTGGAYEVKQHRFFRSLDWNSLLRQKAEFIPQLESEDDTSYFDTRSEKYHHMETEEEDDTNDEDFNVEIRQFSSCSHRFSKVFSSIDRITQNSAEEKEDSVDKTKSTTLPSTETLSWSSEYSEMQQLSTSNSSDTESNRHKLSSGLLPKLAISTEGEQDEAASCPGDPHEEPGKPALPPEECAQEEPEVTTPASTISSSTSDMFAVSPLGSPMSPHSLSSDPSSSRDSSPSRDSSAASASPHQPIVIHSSGKNYGFTIRAIRVYVGDSDIYTVHHIVWNVEEGSPACQAGLKAGDLITHINGEPVHGLVHTEVIELLLKSGNKVSITTTPFENTSIKTGPARRNSYKSRMVRRSKKSKKKESLERRRSLFKKLAKQPSPLLHTSRSFSCLNRSLSSGESLPGSPTHSLSPRSPTPSYRSTPDFPSGTNSSQSSSPSSSANSPAGSGHIRPSTLHGLAPKLGGQRYRSGRRKSAGNIPLSPLARTPSPTPQPTSPQRSPSPLLGHSLGNSKIAQAFPSKMHSPPTIVRHIVRPKSAEPPRSPLLKRVQSEEKLSPSYGSDKKHLCSRKHSLEVTQEEVQREQSQREAPLQSLDENVCDVPPLSRARPVEQGCLKRPVSRKVGRQESVDDLDRDKLKAKVVVKKADGFPEKQESHQKSHGPGSDLENFALFKLEEREKKVYPKAVERSSTFENKASMQEAPPLGSLLKDALHKQASVRASEGAMSDGRVPAEHRQGGGDFRRAPAPGTLQDGLCHSLDRGISGKGEGTEKSSQAKELLRCEKLDSKLANIDYLRKKMSLEDKEDNLCPVLKPKMTAGSHECLPGNPVRPTGGQQEPPPASESRAFVSSTHAAQMSAVSFVPLKALTGRVDSGTEKPGLVAPESPVRKSPSEYKLEGRSVSCLKPIEGTLDIALLSGPQASKTELPSPESAQSPSPSGDVRASVPPVLPSSSGKKNDTTSARELSPSSLKMNKSYLLEPWFLPPSRGLQNSPAVSLPDPEFKRDRKGPHPTARSPGTVMESNPQQREGSSPKHQDHTTDPKLLTCLGQNLHSPDLARPRCPLPPEASPSREKPGLRESSERGPPTARSERSAARADTCREPSMELCFPETAKTSDNSKNLLSVGRTHPDFYTQTQAMEKAWAPGGKTNHKDGPGEARPPPRDNSSLHSAGIPCEKELGKVRRGVEPKPEALLARRSLQPPGIESEKSEKLSSFPSLQKDGAKEPERKEQPLQRHPSSIPPPPLTAKDLSSPAARQHCSSPSHASGREPGAKPSTAEPSSSPQDPPKPVAAHSESSSHKPRPGPDPGPPKTKHPDRSLSSQKPSVGATKGKEPATQSLGGSSREGKGHSKSGPDVFPATPGSQNKASDGIGQGEGGPSVPLHTDRAPLDAKPQPTSGGRPLEVLEKPVHLPRPGHPGPSEPADQKLSAVGEKQTLSPKHPKPSTVKDCPTLCKQTDNRQTDKSPSQPAANTDRRAEGKKCTEALYAPAEGDKLEAGLSFVHSENRLKGAERPAAGVGKGFPEARGKGPGPQKPPTEADKPNGMKRSPSATGQSSFRSTALPEKSLSCSSSFPETRAGVREASAASSDTSSAKAAGGMLELPAPSNRDHRKAQPAGEGRTHMTKSDSLPSFRVSTLPLESHHPDPNTMGGASHRDRALSVTATVGETKGKDPAPAQPPPARKQNVGRDVTKPSPAPNTDRPISLSNEKDFVVRQRRGKESLRSSPHKKAL

SEQ ID NO: 11 - protein, 2362 aa, Homo sapiens; identical to SEQ ID NO: 5

SEQ ID NO: 12 - protein, 2355 aa, Homo sapiens
MDESSILRRRGLQKELSLPRRGSFCRTSNRKSLIGNGQSPALPRPHSPLSAHAGNSPQDSPRNFSPSASAHFSFARRTDGRRWSLASLPSSGYGTNTPSSTVSSSCSSQEKLHQLPYQPTPDELHFLSKHFCTTESIATENRCRNTPMRPRSRSLSPGRSPACCDHEIIMMNHVYKERFPKATAQMEERLKEIITSYSPDNVLPLADGVLSFTHHQIIELARDCLDKSHQGLITSRYFLELQHKLDKLLQEAHDRSESGELAFIKQLVRKILIVIARPARLLECLEFDPEEFYYLLEAAEGHAKEGQGIKTDIPRYIISQLGLNKDPLEEMAHLGNYDSGTAETPETDESVSSSNASLKLRRKPRESDFETIKLISNGAYGAVYFVRHKESRQRFAMKKINKQNLILRNQIQQAFVERDILTFAENPFVVSMYCSFETRRHLCMVMEYVEGGDCATLMKNMGPLPVDMARMYFAETVLALEYLHNYGIVHRDLKPDNLLVTSMGHIKLTDFGLSKVGLMSMTTNLYEGHIEKDAREFLDKQVCGTPEYIAPEVILRQGYGKPVDWWAMGIILYEFLVGCVPFFGDTPEELFGQVISDEINWPEKDEAPPPDAQDLITLLLRQNPLERLGTGGAYEVKQHRFFRSLDWNSLLRQKAEFIPQLESEDDTSYFDTRSEKYHHMETEEEDDTNDEDFNVEIRQFSSCSHRFSKVFSSIDRITQNSAEEKEDSVDKTKSTTLPSTETLSWSSEYSEMQQLSTSNSSDTESNRHKLSSGLLPKLAISTEGEQDEAASCPGDPHEEPGKPALPPEECAQEEPEVTTPASTISSSTLSDMFAVSPLGSPMSPHSLSSDPSSSRDSSPSRDSSAASASPHQPIVIHSSGKNYGFTIRAIRVYVGDSDIYTVHHIVWNVEEGSPACQAGLKAGDLITHINGEPVHGLVHTEVIELLLKSGNKVSITTTPFENTSIKTGPARRNSYKSRMVRRSKKSKKKESLERRRSLFKKLAKQPSPLLHTSRSFSCLNRSLSSGESLPGSPTHSLSPRSPTPSYRSTPDFPSGTNSSQSSSPSSSAPNSPAGSGHIRPSTLHGLAPKLGGQRYRSGRRKSAGNIPLSPLARTPSPTPQPTSPQRSPSPLLGHSLGNSKIAQAFPSKMHSPPTIVRHIVRPKSAEPPRSPLLKRVQSEEKLSPSYGSDKKHLCSRKHSLEVTQEEVQREQSQREAPLQSLDENVCDVPPLSRARPVEQGCLKRPVSRKVGRQESVDDLDRDKLKAKVVVKKADGFPEKQESHQKSHGPGSDLENFALFKLEEREKKVYPKAVERSSTFENKASMQEAPPLGSLLKDALHKQASVRASEGAMSDGRVPAEHRQGGGDFRRAPAPGTLQDGLCHSLDRGISGKGEGTEKSSQAKELLRCEKLDSKLANIDYLRKKMSLEDKEDNLCPVLKPKMTAGSHECLPGNPVRPTGGQQEPPPASESRAFVSSTHAAQMSAVSFVPLKALTGRVDSGTEKPGLVAPESPVRKSPSEYKLEGRSVSCLKPIEGTLDIALLSGPQASKTELPSPESAQSPSPSGDVRASVPPVLPSSSGKKNDTTSARELSPSSLKMNKSYLLEPWFLPPSRGLQNSPAVSLPDPEFKRDRKGPHPTARSPGTVMESNPQQREGSSPKHQDHTTDPKLLTCLGQNLHSPDLARPRCPLPPEASPSREKPGLRESSERGPPTARSERSAARADTCREPSMELCFPETAKTSDNSKNLLSVGRTHPDFYTQTQAMEKAWAPGGKTNHKDGPGEARPPPRDNSSLHSAGIPCEKELGKVRRGVEPKPEALLARRSLQPPGIESEKSEKLSSFPSLQKDGAKEPERKEQPLQRHPSSIPPPPLTAKDLSSPAARQHCSSPSHASGREPGAKPSTAEPSSSPQDPPKPVAAHSESSSHKPRPGPDPGPPKTKHPDRSLSSQKPSVGATKGKEPATQSLGGSSREGKGHSKSGPDVFPATPGSQNKASDGIGQGEGGPSVPLHTDRAPLDAKPQPTSGGRPLEVLEKPVHLPRPGHPGPSEPADQKLSAVGEKQTLSPKHPKPSTVKDCPTLCKQTDNRQTDKSPSQPAANTDRRAEGKKCTEALYAPAEGDKLEAGLSFVHSENRLKGAERPAAGVGKGFPEARGKGPGPQKPPTEADKPNGMKRSPSATGQSSFRSTALPEKSLSCSSSFPETRAGVREASAASSDTSSAKAAGGMLELPAPSNRDHRKAQPAGEGRTHMTKSDSLPSFRVSTLPLESHHPDPNTMGGASHRDRALSVTATVGETKGKDPAPAQPPPARKQNVGRDVTKPSPAPNTDRPISLSNEKDFVVRQRRGKESLRSSPHKKAL

SEQ ID NO: 13 - DNA, 20 bp, synthetic construct
  source 1..20: mol_type = other DNA
ggaaactctgtcggaggaag

SEQ ID NO: 14 - DNA, 20 bp, synthetic construct
  source 1..20: mol_type = other DNA
ggcacaaagagtcccgccag

SEQ ID NO: 15 - DNA, 20 bp, synthetic construct
  source 1..20: mol_type = other DNA
gtagggactccacgctccag

SEQ ID NO: 16 - DNA, 19 bp, synthetic construct
  source 1..19: mol_type = other DNA
ccggaccctagtctcttcg

SEQ ID NO: 17 - DNA, 20 bp, synthetic construct
  source 1..20: mol_type = other DNA
gggttctctgcgaaagtcag

SEQ ID NO: 18 - DNA, 20 bp, synthetic construct
  source 1..20: mol_type = other DNA
atccctgtgttccgtttcag

SEQ ID NO: 19 - DNA, 20 bp, synthetic construct
  source 1..20: mol_type = other DNA
taccctgccgctgccgcacc

SEQ ID NO: 20 - DNA, 20 bp, synthetic construct
  source 1..20: mol_type = other DNA
agcaacccagatgtggcctg

SEQ ID NO: 21 - DNA, 38 bp, synthetic construct
  source 1..38: mol_type = other DNA
taatacgactcactatagagcaaccggaaaagcttaat

SEQ ID NO: 22 - DNA, 35 bp, synthetic construct
  source 1..35: mol_type = other DNA
ttctagctctaaaacattaagcttttccggttgct

SEQ ID NO: 23 - DNA, 58 bp, synthetic construct
  source 1..58: mol_type = other DNA
ccggcccagttgatatggccagaatctcgagattctggccatatcaactgggtttttg

SEQ ID NO: 24 - DNA, 58 bp, synthetic construct
  source 1..58: mol_type = other DNA
ccggccgaagtttctcctgcttaaactcgagtttaagcaggagaaacttcggtttttg

SEQ ID NO: 25 - DNA, 20 bp, synthetic construct
  source 1..20: mol_type = other DNA
aaccctgcccgtatttattt

SEQ ID NO: 26 - DNA, 18 bp, synthetic construct
  source 1..18: mol_type = other DNA
tgtgcattgtgggagagg

SEQ ID NO: 27 - DNA, 22 bp, synthetic construct
  source 1..22: mol_type = other DNA
tgctgacactttattttgctct

SEQ ID NO: 28 - DNA, 20 bp, synthetic construct
  source 1..20: mol_type = other DNA
catctccaagcctctttctg

SEQ ID NO: 29 - protein, 10 aa, synthetic construct
ILTFAENPFV

SEQ ID NO: 30 - protein, 10 aa, synthetic construct
VSMYCSFETR

SEQ ID NO: 31 - protein, 10 aa, synthetic construct
RHLCMVMEYV

SEQ ID NO: 32 - DNA, 10 bp, synthetic construct
  source 1..10: mol_type = other DNA
gccgcacccg

SEQ ID NO: 33 - DNA, 10 bp, synthetic construct
  source 1..10: mol_type = other DNA
gccgcccggg

SEQ ID NO: 34 - DNA, 44 bp, synthetic construct
  source 1..44: mol_type = other DNA
agcaacccagatgtggcctgcgcctcggaaaatcactgaagtac

SEQ ID NO: 35 - DNA, 38 bp, synthetic construct
  source 1..38: mol_type = other DNA
agcaacccagatgtggcctcggaaaatcactgaagtac

SEQ ID NO: 36 - DNA, 46 bp, synthetic construct
  source 1..46: mol_type = other DNA
agcaacccagatgtggcctgcggccctcggaaaatcactgaagtac

SEQ ID NO: 37 - DNA, 119 bp, synthetic construct
  source 1..119: mol_type = other DNA
atcctctggctcggaaactctgtcggaggaaggggagcccagccgcttctcctgcaggtcgcagccgccgcggccgccgggcggcgccctgggaacccggctacccgccgcgtgggctc

SEQ ID NO: 38 - DNA, 48 bp, synthetic construct
  source 1..48: mol_type = other DNA
atcctctggctcggaaactgggaacccggctacccgccgcgtgggctc

SEQ ID NO: 39 - protein, 93 aa, synthetic construct
MGEKVSEAPEPVPRGCSGHGARTLVSSAAAVSSEGASSAESSSGSETLSEEGEPSRFSCRSQPPRPPGGALGTRLPAAWAPARVALERGVPTL

SEQ ID NO: 40 - protein, 93 aa, synthetic construct
MGEKVSEAPEPVPRGCSGHGARTLVSSAAAVSSEGASSAESSSGSETGNPATRRVGSRARGSGAWSPYPAAAAPGRSGAAGAPGQQRIPRGAG

SEQ ID NO: 41 - DNA, 25 bp, synthetic construct
  source 1..25: mol_type = other DNA
caaagagtcccgccagaggtttgcc

SEQ ID NO: 42 - DNA, 22 bp, synthetic construct
  source 1..22: mol_type = other DNA
caaagagtcccagaggtttgcc

SEQ ID NO: 43 - DNA, 14 bp, synthetic construct
  source 1..14: mol_type = other DNA
agtcccgccaaagg

SEQ ID NO: 44 - DNA, 11 bp, synthetic construct
  source 1..11: mol_type = other DNA
agtcccaaagg